SEQUENCE LISTING (13 sequences)

SEQ ID NO: 1 - DNA, 1626 bp, Homo sapiens
  source 1..1626: mol_type = genomic DNA
ctccttccctgtctctgcctctccctcccttcctcaggcatcagagcggagacttcagggagaccagagcccagcttgccaggcactgagctagaagccctgccatggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtaggagtcagagcccacatggccgggccctctgtagctccctcctcccatctccttcccccagcccaataaaggtcccttagccccga

SEQ ID NO: 2 - DNA, 1512 bp, Mus musculus
  source 1..1512: mol_type = genomic DNA
atggcacccctgaggccctttttcatactagccctggtggcatgggtttctctggctgaccaagagtcatgcaagggccgctgcactcagggtttcatggccagcaagaagtgtcagtgtgacgagctttgcacttactatcagagctgctgtgccgactacatggagcagtgcaagccccaagtaacgcggggggacgtgttcactatgccagaggatgattattggagctatgactacgtggaggagcccaagaacaataccaacaccggtgtgcaacccgagaacacctctccacccggtgacctaaatcctcggacggacggcactctaaagccgacagccttcctagatcctgaggaacagccaagcaccccagcgcctaaagtggagcaacaggaggagatcctaaggcccgacaccactgatcaagggacccctgagtttccagaggaagaactgtgcagtggaaagccctttgacgccttcacggatctcaagaatgggtccctctttgccttccgagggcagtactgctatgagctagatgagacggcagtgaggcctgggtaccccaaacttatccaagatgtctggggcattgagggccccatcgatgctgccttcactcgcatcaactgtcaggggaagacctacttgttcaagggtagtcagtactggcgctttgaggatggggtcctggaccctggttatccccgaaacatctccgaaggcttcagtggcataccagacaatgttgatgcagcgttcgcccttcctgcccaccgttacagtggccgggaaagggtctacttcttcaaggggaagcagtactgggagtacgaatttcagcagcaacccagccaggaggagtgcgaaggcagctctctgtcagccgtgtttgagcactttgccttgcttcagcgggacagctgggagaacattttcgaactcctcttctggggcagatcctctgatggagccagagaaccccaattcatcagccggaactggcatggtgtgccagggaaagtggacgctgctatggccggccgcatctacgtcactggctccttatcccactctgcccaagccaaaaaacagaagtctaagcgtagaagccgaaagcgctatcgttcacgccgaggccacagacgcagccagagctcgaactcccgtcgttcatcacgttcaatctggttctctttgttctccagcgaggagagtgggctaggaaccaaaaacaactatgattatgatatggactggcttgtacctgccacctgcgagcccattcagagcgtctatttcttctctggagacaaatactaccgagtcaaccttagaacccggcgagtggactctgtgaatcctccctacccacgctccattgctcagtattggctgggctgcccgacctctgagaagtaggaatcagagcccactcggctgagcttcaggagcctcatctctttctcccagcccaataaaaagtctgttggctacgaaaaa

SEQ ID NO: 3 - DNA, 48 bp, synthetic construct
  source 1..48: mol_type = other DNA
cccagcagggagtcgtatttactcgagtaaatacgactccctgctggg

SEQ ID NO: 4 - DNA, 48 bp, synthetic construct
  source 1..48: mol_type = other DNA
atcaacttggaaccataattactcgagtaattatggttccaagttgat

SEQ ID NO: 5 - DNA, 4250 bp, Homo sapiens
  source 1..4250: mol_type = genomic DNA
attcgcctctgggaggtttaggaagcggctccgggtcggtggccccaggacagggaagagcgggcgctatggggagccggacgccagagtcccctctccacgccgtgcagctgcgctggggcccccggcgccgacccccgctgctgccgctgctgttgctgctgctgccgccgccacccagggtcgggggcttcaacttagacgcggaggccccagcagtactctcggggcccccgggctccttcttcggattctcagtggagttttaccggccgggaacagacggggtcagtgtgctggtgggagcacccaaggctaataccagccagccaggagtgctgcagggtggtgctgtctacctctgtccttggggtgccagccccacacagtgcacccccattgaatttgacagcaaaggctctcggctcctggagtcctcactgtccagctcagagggagaggagcctgtggagtacaagtccttgcagtggttcggggcaacagttcgagcccatggctcctccatcttggcatgcgctccactgtacagctggcgcacagagaaggagccactgagcgaccccgtgggcacctgctacctctccacagataacttcacccgaattctggagtatgcaccctgccgctcagatttcagctgggcagcaggacagggttactgccaaggaggcttcagtgccgagttcaccaagactggccgtgtggttttaggtggaccaggaagctatttctggcaaggccagatcctgtctgccactcaggagcagattgcagaatcttattaccccgagtacctgatcaacctggttcaggggcagctgcagactcgccaggccagttccatctatgatgacagctacctaggatactctgtggctgttggtgaattcagtggtgatgacacagaagactttgttgctggtgtgcccaaagggaacctcacttacggctatgtcaccatccttaatggctcagacattcgatccctctacaacttctcaggggaacagatggcctcctactttggctatgcagtggccgccacagacgtcaatggggacgggctggatgacttgctggtgggggcacccctgctcatggatcggacccctgacgggcggcctcaggaggtgggcagggtctacgtctacctgcagcacccagccggcatagagcccacgcccacccttaccctcactggccatgatgagtttggccgatttggcagctccttgacccccctgggggacctggaccaggatggctacaatgatgtggccatcggggctccctttggtggggagacccagcagggagtagtgtttgtatttcctgggggcccaggagggctgggctctaagccttcccaggttctgcagcccctgtgggcagccagccacaccccagacttctttggctctgcccttcgaggaggccgagacctggatggcaatggatatcctgatctgattgtggggtcctttggtgtggacaaggctgtggtatacaggggccgccccatcgtgtccgctagtgcctccctcaccatcttccccgccatgttcaacccagaggagcggagctgcagcttagaggggaaccctgtggcctgcatcaaccttagcttctgcctcaatgcttctggaaaacacgttgctgactccattggtttcacagtggaacttcagctggactggcagaagcagaagggaggggtacggcgggcactgttcctggcctccaggcaggcaaccctgacccagaccctgctcatccagaatggggctcgagaggattgcagagagatgaagatctacctcaggaacgagtcagaatttcgagacaaactctcgccgattcacatcgctctcaacttctccttggacccccaagccccagtggacagccacggcctcaggccagccctacattatcagagcaagagccggatagaggacaaggctcagatcttgctggactgtggagaagacaacatctgtgtgcctgacctgcagctggaagtgtttggggagcagaaccatgtgtacctgggtgacaagaatgccctgaacctcactttccatgcccagaatgtgggtgagggtggcgcctatgaggctgagcttcgggtcaccgcccctccagaggctgagtactcaggactcgtcagacacccagggaacttctccagcctgagctgtgactactttgccgtgaaccagagccgcctgctggtgtgtgacctgggcaaccccatgaaggcaggagccagtctgtggggtggccttcggtttacagtccctcatctccgggacactaagaaaaccatccagtttgacttccagatcctcagcaagaatctcaacaactcgcaaagcgacgtggtttcctttcggctctccgtggaggctcaggcccaggtcaccctgaacggtgtctccaagcctgaggcagtgctattcccagtaagcgactggcatccccgagaccagcctcagaaggaggaggacctgggacctgctgtccaccatgtctatgagctcatcaaccaaggccccagctccattagccagggtgtgctggaactcagctgtccccaggctctggaaggtcagcagctcctatatgtgaccagagttacgggactcaactgcaccaccaatcaccccattaacccaaagggcctggagttggatcccgagggttccctgcaccaccagcaaaaacgggaagctccaagccgcagctctgcttcctcgggacctcagatcctgaaatgcccggaggctgagtgtttcaggctgcgctgtgagctcgggcccctgcaccaacaagagagccaaagtctgcagttgcatttccgagtctgggccaagactttcttgcagcgggagcaccagccatttagcctgcagtgtgaggctgtgtacaaagccctgaagatgccctaccgaatcctgcctcggcagctgccccaaaaagagcgtcaggtggccacagctgtgcaatggaccaaggcagaaggcagctatggcgtcccactgtggatcatcatcctagccatcctgtttggcctcctgctcctaggtctactcatctacatcctctacaagcttggattcttcaaacgctccctcccatatggcaccgccatggaaaaagctcagctcaagcctccagccacctctgatgcctgagtcctcccaatttcagactcccattcctgaagaaccagtccccccaccctcattctactgaaaaggaggggtctgggtacttcttgaaggtgctgacggccagggagaagctcctctccccagcccagagacatacttgaagggccagagccaggggggtgaggagctggggatccctcccccccatgcactgtgaaggacccttgtttacacataccctcttcatggatgggggaactcagatccagggacagaggccccagcctccctgaagcctttgcattttggagagtttcctgaaacaacttggaaagataactaggaaatccattcacagttctttgggccagacatgccacaaggacttcctgtccagctccaacctgcaaagatctgtcctcagccttgccagagatccaaaagaagcccccagctaagaacctggaacttggggagttaagacctggcagctctggacagccccaccctggtgggccaacaaagaacactaactatgcatggtgccccaggaccagctcaggacagatgccacacaaggatagatgctggcccagggcccagagcccagctccaaggggaatcagaactcaaatggggccagatccagcctggggtctggagttgatctggaacccagactcagacattggcacctaatccaggcagatccaggactatatttgggcctgctccagacctgatcctggaggcccagttcaccctgatttaggagaagccaggaatttcccaggaccctgaaggggccatgatggcaacagatctggaacctcagcctggccagacacaggccctccctgttccccagagaaaggggagcccactgtcctgggcctgcagaatttgggttctgcctgccagctgcactgatgctgcccctcatctctctgcccaacccttccctcaccttggcaccagacacccaggacttatttaaactctgttgcaagtgcaataaatctgacccagtgcccccactgaccagaactagaa

SEQ ID NO: 6 - DNA, 4440 bp, Mus musculus
  source 1..4440: mol_type = genomic DNA
agtctgagccaggccagaccaccggcctccagctggggccggagccagggttcagaggtgcggccccccacgccccttcgggtgcggggcgcggcctcggcgtccccgggacccaggaatgcccccccgccagccggcccggcggccggggggagggcccagccgggagtttggcaaactcctccccgcgttgagtcattcgcctctgggaggtttaggaagcggctccgggtcggtggccccaggacagggaagagcgggcactatggggagctggacgccacggtcgcctcgatctcctctccacgcggtgctgctgcgctgggggccccgacgcctaccgccgctgctgcctctgctgctgctactgtggccgccaccactccaggttgggggcttcaacctagacgcggaggccccggcggtgctctccgggccccccggctccctttttggcttctccgtggagttttaccggccgggaagggacggagtcagtgtgctggttggggcacccaaggctaacactagccagccaggtgtactgcaaggtggtgctgtctatgtgtgtccctggggcaccagtcctatccagtgcaccaccattcaatttgacagcaaaggctcccggattctggagtcctcactgtacagtgccaagggagaggagcctgtggagtacaagtccttgcagtggttcggagcaacagttcgggcccatggctcctccatcttggcatgtgctccactgtatagctggcgcacagaaaaggacccacagaatgacccagtgggcacctgctacctctccacagaaaacttcacccggattctggagtacgcaccttgccgctcagattttggcagtgcagcagggcagggctactgccaagggggcttcagtgctgagttcaccaagactggccgtgtggtcctgggtggacctggaagctacttctggcaaggccagatcctgtccgccactcaagagcagatctcggagtcctattacccagagtatctcatcaaccctgttcaggggcagctgcagacccgccaggccagctccgtctatgatgacagctacttgggatactctgtggctgtgggtgaattcagtggtgatgacacagaagactttgttgctggcgtgcccaaggggaacctcacctatggctatgtcaccgtccttaatggctcagacatccactccctctacaacgtctcaggagaacagatggcctcctacttcggctatgctgtggctgccactgataccaatggagatgggctagatgacctactggtaggggcacccctgctcatggagcggacagctgatgggagacctcaggaggtgggcagggtctacatctatctgcagcgcccagcgggcatagatcccacacccaccctcaccctcactgggcaagatgagttcagccgattcggcagctccttgacacccttgggggacctggaccaagacggctacaatgatgtcgccattggggctccatttggtggggaggcccagcagggagtcgtatttatattcccgggaggcccaggaggactgagcactaaaccttcccaggttttgcagcctctttgggcagctggccgtaccccagacttctttggctctgcccttcgaggaggacgagatctggatggcaatggataccctgatctaatcgttggatcctttggtgtggacaaggctctggtgtacagagggcggcccatcatatctgccagcgcatctctcaccatcttcccctccatgttcaacccagaggagcgcagttgcagcctggaagggaaccctgtgtcctgcatcaaccttagcttctgcctcaatgcctctggaaaacatgtccccaactctataggcttcgaggtggaactccaactggactggcagaagcaaaagggaggggtccggcgggcactgttcctgacttccaagcaggccacccttacccagaccctgcttatccagaatggggctcgggaggactgcagggagatgaagatctacctcaggaatgaatcagaattcagagacaaactctccccaattcacattgccctcaacttctccttggaccccaaagctcccatggacagccatggcctccggccagttctacactaccaaagcaaaagcaggatagaggacaaggcccagatcttgctggactgtggtgaagacaatatctgtgtgcctgacctgcagctggatgtgtatggggagaagaaacatgtgtacctgggtgacaagaacgcactgaacctgacattccatgcccaaaatctgggtgagggcggtgcctatgaagccgagcttcgggtcacagcccctctagaggccgagtactcaggacttgtcagacacccagggaacttctccagcctgagctgtgactactttgctgtgaaccagagccgccagctggtgtgtgacctgggcaaccccatgaaggcaggcaccagtctctggggtggccttcggttcactgttcctcatcttcaagacacaaagaaaaccatccagtttgactttcagatcctcagcaagaacctgaacaactcacaaagcaacgtggtctccttcccactctcggtggaggctcaagcccaggtctcccttaatggtgtctccaagcctgaagctgtgattttcccagtcagcgactggaatcctcaagaccagcctcagaaggaggaagacttgggcccagctgtccaccatgtctacgagctcatcaaccaggggcccagctccatcagccagggtgtgctggagctcagctgtccacaggctctggaaggccaacagctcctctatgtgaccaaggtgacaggactcagcaactgcacctccaactacacccccaactcacagggcctggagttggatccagagacctctccacaccacctgcagaaacgagaggctccagggaggagttctactgcctcaggaacacaagttctgaaatgccctgaagccaagtgtttcaggctgcgctgtgagtttgggccactgcaccgccaagagagccgtagtctgcagctgcatttccgagtctgggccaagaccttcttgcagcgggaataccagccatttagccttcagtgtgaggctgtatatgaagctctgaagatgccctaccagatcctgcctcggcagcttccccaaaagaaacttcaggtggccacagccgtgcagtggaccaaggcagaaggcagcaatggtgtcccgttgtggatcatcatcctagccattctttttggcctcctgctcctaggtctgctcatctacgtcctctacaagctcggcttcttcaaacgttccctcccctacggcacagccatggaaaaagctcagctcaagcctccagccacctcagatgcctgagccctcctgatctcagactcatgatcccgaagagccagtccaataccttccgacagggagggactaggcacttcctgaaggtgctgagggctggggagaagctcttcttcctagcccagagacatacttgaagggccagagctggggggcaagaagcttgtggatccttccccccatgcactgtgaaggaccctcgtttacacatgccctctcgtggatgggggcctcaggtccagggacagaagctccagctttccttcagcctttgcattttggagacttttctgaaatcaacttggaaccataattagggaatccagttgtggttctctgggccgggcatgccaacaggacttcccatctgactccggcctgcaaagatgcatctttggtcttgccagagaaccaaaggaagtctgcagctaagagcctgagacttggggagtgagacctggcaactccaacagcccccatcctggaaggccaatgaagaacactaaccgtggatggtgctgcaggaccagctcaggacaaatcccaaacaaaggtcgatgctggcccagacccagatccatggaagtcagaaagagaatggagccagacccagtctggggcctagggttgatctggagcccagtctcggattctaagaacctatgctagcagacccaaggtggcatttgggtcagctgcagacctgggcttagaaacctatttatccctgacttaggacaagtcaggaattgcccaggaccctaaccaggccacattggcaaacagacctggaacctcagcctggccactcattccccagagagaacgggaagccccaggcctgtatgacctgggttctgcccaccagctgcactgacgctgcccctctctccctagccaaccctcccctctcctccagaacccaccccaacttatttaaactctgttgcaagtgcaataaaccccacccactgcccccactgaccaaaaaaaaaaaaaaaaaaaaaaaaaaaaaaaaa

SEQ ID NO: 7 - RNA, 21 nt, synthetic construct
  source 1..21: mol_type = other RNA
  modified_base 20..21: mod_base = OTHER; note = thymine
tcggcttctacgcttagactt

SEQ ID NO: 8 - RNA, 21 nt, synthetic construct
  source 1..21: mol_type = other RNA
  modified_base 21: mod_base = OTHER; note = thymine
ataatcatagttgttgtaggt

SEQ ID NO: 9 - RNA, 21 nt, synthetic construct
  source 1..21: mol_type = other RNA
  modified_base 20..21: mod_base = OTHER; note = thymine
gtctaagcgtagaagccgatt

SEQ ID NO: 10 - RNA, 21 nt, synthetic construct
  source 1..21: mol_type = other RNA
  modified_base 20..21: mod_base = OTHER; note = thymine
ctacaacaactatgattattt

SEQ ID NO: 11 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 12 - protein, 476 aa, Mus musculus
MAPLRPFFILALVAWVSLADQESCKGRCTQGFMASKKCQCDELCTYYQSCCADYMEQCKPQVTRGDVFTMPEDDYWSYDYVEEPKNNTNTGVQPENTSPPGDLNPRTDGTLKPTAFLDPEEQPSTPAPKVEQQEEILRPDTTDQGTPEFPEEELCSGKPFDAFTDLKNGSLFAFRGQYCYELDETAVRPGYPKLIQDVWGIEGPIDAAFTRINCQGKTYLFKGSQYWRFEDGVLDPGYPRNISEGFSGIPDNVDAAFALPAHRYSGRERVYFFKGKQYWEYEFQQQPSQEECEGSSLSAVFEHFALLQRDSWENIFELLFWGRSSDGAREPQFISRNWHGVPGKVDAAMAGRIYVTGSLSHSAQAKKQKSKRRSRKRYRSRRGHRRSQSSNSRRSSRSIWFSLFSSEESGLGTKNNYDYDMDWLVPATCEPIQSVYFFSGDKYYRVNLRTRRVDSVNPPYPRSIAQYWLGCPTSEK

SEQ ID NO: 13 - DNA, 48 bp, synthetic construct
  source 1..48: mol_type = other DNA
cctaaggttaagtcgccctcgctcgagcgagggcgacttaaccttagg

(19) United States
(12) Patent Application Publication
(10) Pub. No.: US 2023/0383295 A1
(43) Pub. Date: Nov. 30, 2023

(54) METHODS FOR REGULATING BLOOD-CENTRAL NERVOUS SYSTEM (BLOOD-CNS) BARRIER AND USES THEREOF

(71) Applicant: President and Fellows of Harvard College, Cambridge, MA (US)

(72) Inventors: Swathi Ayloo, Cambridge, MA (US); Chenghua Gu, Cambridge, MA (US)

(73) Assignee: President and Fellows of Harvard College, Cambridge, MA (US)

(21) Appl. No.: 18/296,350

(22) Filed: Apr. 5, 2023

Related U.S. Application Data
(60) Provisional application No. 63/328,122, filed on Apr. 6, 2022.

(51) Int. Cl. C12N 15/113; C07K 16/28; A61P 43/00

(52) U.S. Cl. CPC C12N 15/113; C07K 16/2839; A61P 43/00; C12N 2310/11; C12N 2310/122

(57) ABSTRACT

The present disclosure, at least in part, provides methods for regulating Blood-Central Nervous System (blood-CNS) barrier permeability (e.g., increasing or decreasing Blood-CNS barrier permeability) by regulating signaling between pericyte derived vitronectin and integrin expressed on CNS endothelial cells (e.g., integrin α5). In some aspects, the present disclosure also provides a Blood-Central Nervous System (blood-CNS) barrier model comprising CNS endothelial cells and vitronectin or a plurality of cells secreting vitronectin, and methods for producing the same.

### RELATED APPLICATIONS

This application claims priority under 35 U.S.C. § 119(e) to U.S. Provisional Application, U.S.S.N. 63/328,122, filed Apr. 6, 2022, the entire contents of which is incorporated herein by reference.

### GOVERNMENT SUPPORT

This invention was made with government support under NS116820, NS092473, NS072030, HL153261, and DA048786 awarded by National Institutes of Health (NIH). The government has certain rights in this invention.

### REFERENCE TO AN ELECTRONIC SEQUENCE LISTING

The contents of the electronic sequence listing (H082470409US01-SEQ-LJG.xml; Size: 27,387 bytes; and Date of Creation: Apr. 5, 2023) is herein incorporated by reference in its entirety.

### BACKGROUND

The CNS requires an optimal and tightly regulated microenvironment for efficient synaptic transmission. This is achieved by blood-CNS barriers that regulate substance flux to maintain tissue homeostasis. Barrier properties of CNS endothelial cells require induction and maintenance from brain parenchymal cells. However, the signal(s) to facilitate endothelial cells to maintain barrier integrity remain elusive.

### SUMMARY

The present disclosure, at least in part, provides compositions and the uses thereof, kits and methods for regulating Blood-Central Nervous System (blood-CNS) barrier permeability (e.g., increasing or decreasing Blood-CNS barrier permeability) by regulating signaling between pericyte-derived vitronectin and integrin expressed on CNS endothelial cells (e.g., integrin α5). In some aspects, the present disclosure also provides a Blood-Central Nervous System (blood-CNS) barrier model comprising CNS endothelial cells derived from various sources (e.g., primary CNS endothelial cells, iPSCs, or amnionic fluid stem cells) and vitronectin or a plurality of cells secreting vitronectin, and methods for producing the same.

In some aspects, the present disclosure provides a method for increasing Blood-Central Nervous System (Blood-CNS) Barrier permeability to treat a disease (e.g., amyotrophic lateral sclerosis (ALS), Alzheimer's disease, ataxia, brain and nerve tumors, multiple sclerosis, or muscular dystrophy) in a subject, the method comprising administering to the subject an inhibitor of vitronectin-integrin signaling at the Blood-CNS Barrier. In some embodiments, the blood-CNS barrier is the blood-brain barrier. In other embodiments, the blood-CNS barrier is the blood-retina barrier.

In some embodiments, the vitronectin is secreted by CNS pericytes. In some embodiments, the integrin is expressed on the cell surface of CNS endothelial cells. In some embodiments, the integrin is a, RGD binding integrin. In some embodiments, the integrin is integrin α5.

In some embodiments, the inhibitor of vitronectin-integrin signaling is a vitronectin inhibitor. In some embodiments, the vitronectin inhibitor is capable of inhibiting vitronectin expression and/or activity (e.g., inhibitory nucleic acids targeting VTN, small molecule inhibitors, antibodies target vitronectin, etc.).

In some embodiments, the vitronectin inhibitor is an inhibitory nucleic acid targeting VTN. In some embodiments, the inhibitory nucleic acid targeting vitronectin mRNA is an siRNA, an shRNA, an miRNA, an amiRNA, or an ASO targeting VTN. In some embodiments, the inhibitory nucleic acid targeting vitronectin mRNA is an siRNA. In some embodiments, the inhibitory nucleic acid targeting VTN is delivered to the CNS. In some embodiments, the inhibitory nucleic acid targeting VTN is delivered to CNS pericytes.

In some embodiments, the vitronectin inhibitor is an antibody targeting vitronectin.

In some embodiments, the inhibitor of the vitronectin-integrin signaling is an integrin inhibitor. In some embodiments, the integrin inhibitor is an integrin α5 inhibitor. In some embodiments, the integrin α5 inhibitor is capable of inhibiting integrin α5 expression and/or activity.

In some embodiments, the integrin α5 inhibitor is an inhibitory nucleic acid targeting integrin ITGA5. In some embodiments, the inhibitor nucleic acid targeting integrin ITGA5 mRNA is an siRNA, an shRNA, an miRNA, an amiRNA or an ASO targeting integrin ITGA5. In some embodiments, the inhibitor nucleic acid targeting ITGA5 is a shRNA. In some embodiments, the shRNA targeting IGTA5 comprises the nucleic acid sequence of SEQ ID NO: 5 or 6.

In some embodiments, the inhibitory nucleic acid targeting ITGA5 is delivered to the CNS. In some embodiments, the inhibitory nucleic acid targeting ITGA5 is delivered to the CNS endothelial cells.

In some embodiments, the integrin α5 inhibitor is an antibody targeting integrin α5.

In some embodiments, the integrin α5 inhibitor is a peptide containing RGD.

In some embodiments, the integrin α5 inhibitor is a non-peptidic RGD mimic.

In some embodiments, administration of a vitronectin-integrin signaling inhibitor increase blood-CNS barrier such that a therapeutic agent can be delivered to the CNS. In some embodiments, the method comprises delivering to the subject a vitronectin-integrin signaling inhibitor and a therapeutic agent.

In some embodiments, the disease is wherein the disease is a neuromuscular disease, neurodegenerative disease, brain and nerve tumors, Neurogenetic Diseases, Cognitive disorders, Familial dystonia, Neuroinfectious disease, neuropsychiatric disorders.

In some aspects, the present disclosure provides methods for decreasing Blood-Central Nervous System (Blood-CNS) Barrier permeability for treating a disease in a subject, the method comprising administering to the subject an agent that promotes vitronectin-integrin signaling at the blood-CNS barrier.

In some embodiments, the agent that promotes vitronectin-integrin signaling is recombinant vitronectin. In some embodiments, the agent that promotes vitronectin-integrin signaling is a nucleic acid encoding vitronectin.

In some embodiments, the nucleic acid encoding vitronectin is delivered to the CNS. In some embodiments, the nucleic acid encoding vitronectin is delivered to CNS pericyte.

In some embodiments, the integrin is integrin α5. In some embodiments, the agent that promotes integrin signaling is a nucleic acid encoding integrin α5. In some embodiments, the nucleic acid encoding integrin α5 is delivered to the CNS. In some embodiments, the nucleic acid encoding integrin α5 is delivered to CNS endothelial cells.

The present disclosure, at least in part, provides a method for decreasing Blood-Central Nervous System (Blood-CNS) Barrier permeability for treating a disease in a subject, the method comprising administering to the subject a focal adhesion kinase (FAK) inhibitor. In some embodiments, the FAK inhibitor is PF-562271. In some embodiments, the subject having a disease in need of decreased blood-CNS barrier (i.e., maintenance of blood-CNS barrier integrity) has or is at risk of having a retinal disease, a neurodegenerative disease, an acute injury of the CNS, a neuroinfectious disease, a primary and metastatic cancers of the CNS, an autoimmune disease of the CNS, a neuroinflammatory conditions, or a cognitive disorder. In some embodiments, the retinal disease is diabetic retinopathy. In some embodiments, the neurodegenerative disease is Huntington's disease. In some embodiments, the acute injury of the CNS is stroke or head trauma. In some embodiments, the Neuroinfectious disease is encephalitis, sepsis, or COVID-19. In some embodiments, the primary cancer of the CNS is glioblastoma, meningioma, or lymphoma. In some embodiments, the metastatic cancer of the CNS is lung cancer, metastatic breast cancer, or melanoma. In some embodiments, the autoimmune disease of the CNS is multiple sclerosis. In some embodiments, the neuroinflammatory condition is CNS Lupus, CNS Lyme Disease, Neurosarcoidosis, Neuromyelitis optica (NMO), or Paraneoplastic and Autoimmune Encephalitis. In some embodiments, the cognitive disorder is dementia resulting from Alzheimer's disease, Lewy body dementia, frontotemporal dementia, encephalopathy, or post-acute COVID syndrome.

In some aspects, the present disclosure provides a blood-CNS barrier model comprising: (i) a plurality of CNS endothelial cells; and (ii) vitronectin. In some aspects, the present disclosure provides a blood-CNS barrier model comprising: (i) a plurality of CNS endothelial cells; and (ii) a plurality of cells secreting vitronectin. In some embodiments, the cells secreting vitronectin are pericytes.

In some aspects, the present disclosure also provides a method for producing a blood-CNS barrier model, the method comprising culturing a plurality of endothelial progenitor cells in the presence of vitronectin. In some aspects, the present disclosure also provides a method for producing a blood-CNS barrier model, the method comprising culturing a plurality of endothelial progenitor cells in the presence of a plurality of cells secreting vitronectin. In some embodiments, the method comprising treating the endothelial progenitor cells with retinoic acid.

In some aspects, the present disclosure provides the endothelial progenitor cells are iPSC cells, primary brain endothelial cells, or immortalized brain endothelial cells.

The details of one or more embodiments of the invention are set forth in the description below. Other features or advantages of the present invention will be apparent from the following drawing and detailed description of certain embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the Detailed Description of Specific Embodiments presented herein.

FIGS. 1A-1E show that vitronectin expression coincides with functional barrier formation and is enriched in CNS pericytes compared to peripheral tissue pericytes. FIGS. 1A-1B show the immunostaining of vitronectin and blood vessels (isolectin) in retina (FIG. 1A) and (FIG. 1B) brain of a P7 mouse. FIG. 1C shows the in situ hybridization for Vtn, and Pdgfrb (pericyte gene) and immunostaining for PECAM1 (vessel marker) with DAPI (nuclei) in brain of a P7 mouse. Dashed lines indicate pericyte nucleus and vessel outline. FIG. 1D shows the in situ hybridization for Vtn and Pdgfrb in brain and lung of a P7 mouse. FIG. 1E shows a scatter plot between Pdgfrb and Vtn in brain and lung. Brain cells show expression of both Vtn and Pdgfrb while lung cells only express Pdgfrb. Each dot represents an individual cell, n=60 and 62 cells, respectively, from N=3 animals. Pearson correlation coefficient r=0.82 for brain and r=0.36 for lung. ]

FIGS. 2A-2E show that vitronectin is essential for blood-CNS barrier integrity. FIG. 2A shows Sulfo-NHS-Biotin (tracer) leaks out of blood vessels (isolectin) in retinas of P10 Vtn^{-/-} mice. Arrowheads show tracer hotspots in Vtn^{-/-} mice, white boxes correspond to higher magnification images in FIG. 2B. FIG. 2B shows Sulfo-NHS-Biotin leaked out in Vtn^{-/-} mice taken up by neuronal cell bodies (arrowheads). FIG. 2C shows the quantification of vessel permeability in retinas of wildtype and Vtn^{-/-} mice. n=5 animals per genotype. Mean±S.D.; ***p<0.001; Student's t-test. FIG. 2D shows the leakage of Sulfo-NHS-Biotin from blood vessels (Icam2) in the cerebellum of P10 Vtn^{-/-} mice. White boxes correspond to higher magnification panels shown. FIG. 2E shows the quantification of vessel permeability in retinas of wildtype and Vtn^{-/-} mice. N=4 animals per genotype. Mean±S.D.; ***p<0.001; Student's t-test.

FIGS. 3A-3H show that vitronectin in plasma is not required for blood-CNS barrier function. FIG. 3A depicts the illustration of the experimental paradigm to knock-down plasma vitronectin specifically with intravenous injections of siRNAs followed by vitronectin protein level measurement in plasma and evaluation of barrier integrity by leakage assays. FIG. 3B shows the validation of the ELISA kit to measure vitronectin protein levels in plasma of wildtype, heterozygotes, and vitronectin null mice. n=2 animals per genotype. Mean±S.D.; **p<0.01, ***p<0.001; one-way ANOVA with Tukey's post hoc test. FIGS. 3C and 3F show the vitronectin levels measured by ELISA in plasma of mice injected with control (Ctrl) siRNA or two independent siRNAs targeting vitronectin. 24 hours post last siRNA injection (FIG. 3C) and 72 hours post last siRNA injection (FIG. 3F). n=3 mice per siRNA in each case. Mean±S.D.; **p<0.01, ***p<0.001; one-way ANOVA with Tukey's post hoc test. At the 24 hour time point, siRNA #1 yields 87.58±1.71% and siRNA #2 yields 94.06±0.83% knockdown of plasma vitronectin. At the 72 hour timepoint, siRNA #1 yields 92.73±0.25% and siRNA #2 yields 98.56±0.06% knockdown of plasma vitronectin. FIGS. 3D and 3E show Sulfo-NHS-Biotin confined to vessels (FIG. 3D) in retinas of mice injected with siRNA targeting vitronectin, 24 hours post last siRNA injection. Corresponding quantification of vessel permeability (FIG. 3E). White boxes correspond to panel of higher magnification images. n=3 mice per siRNA. Mean±S.D.; n.s. not significant, p>0.05; one-way ANOVA with Tukey's post hoc test. FIGS. 3G and 3H show Sulfo-NHS-Biotin confined to vessels (FIG. 3G) in retinas of mice injected with siRNA targeting vitronectin, 72 hours post last siRNA injection. Corresponding quantification of vessel permeability (FIG. 3H). White boxes correspond to panel of higher magnification images. n=3 mice per siRNA. Mean±S.D.; n.s. not significant, p>0.05; one-way ANOVA with Tukey's post hoc test.

FIGS. 4A-4H show that vitronectin regulates blood-CNS barrier function by suppressing transcytosis in CNS endothelial cells. FIGS. 4A-4B show EM images of HRP halting at tight junctions (arrows) in both retinas (FIG. 4A) and cerebellum (FIG. 4B) of wildtype and Vtn^{-/-} mice. Luminal (L) and abluminal sides indicated by dashed lines. FIG. 4C shows Claudin-5 immunostaining in P10 retinas of wildtype and Vtn^{-/-} mice. FIG. 4D shows higher magnification images of Claudin-5 and ZO-1 in P10 retinas to show expression and localization of tight junction proteins at cell-cell junctions. FIGS. 4E and 4G show EM images showing HRP-filled vesicles (arrowheads) in endothelial cells of retinas (FIG. 4E) and cerebellum (FIG. 4G) of wildtype and Vtn^{-/-} mice. FIGS. 4F and 4H show the quantification of tracer-filled vesicles in endothelial cells of retinas (FIG. 4F) and cerebellum (FIG. 4H). n=4 animals per genotype, 15-20 vessels per animal. Mean±S.D.; ***p<0.001, **p<0.01; Student's t-test.

FIGS. 5A-5I show vitronectin is not required for normal vessel patterning or pericyte coverage. FIG. 5A shows tilescan images showing retinal vasculature of P10 retinas in wildtype and Vtn^{-/-} mice. FIGS. 5B-5D show quantification of vessel density (FIG. 5B), capillary branching (FIG. 5C), and radial outgrowth (FIG. 5D) in P10 retinas. n=6 animals per genotype. Mean±S.D.; n.s. not significant, p>0.05; Student's t-test. FIG. 5E shows P10 retinas of NG2:DsRed+ wildtype and Vtn^{-/-} mice immunostained for ERG1/2/3 (endothelial nuclei) and vessels (isolectin). FIGS. 5F and 5G show quantification of pericyte coverage (FIG. 5F) and pericyte density (FIG. 5G) in retinas of wildtype and Vtn^{-/-} mice. n=5 animals per genotype. Mean±S.D.; n.s. not significant, p>0.05; Student's t-test. FIGS. 5H and 5I show representative western blots (FIG. 5H) and quantification (FIG. 5I) of PDGFRβ protein levels normalized to GAPDH in whole retinal lysates of P10 wildtype and Vtn^{-/-} mice. n=3 animals per genotype. Mean±S.D.; n.s. not significant, p>0.05; Student's t-test.

FIGS. 6A-6L show vitronectin binding to integrin receptors is essential for barrier function. FIG. 6A depicts a schematic illustrating binding of ligands containing RGD-motif with integrin receptors. FIG. 6B shows the leakage of Sulfo-NHS-Biotin (tracer) from vessels (isolectin) in P10 Vtn^{RGE} mice. White boxes correspond to higher magnification images shown in FIG. 6C. FIG. 6C shows tracer hotspots (arrowheads) in retinas of Vtn^{RGE} mice. FIG. 6D shows the quantification of vessel permeability in wildtype and Vtn^{RGE} mice. n=5 animals per genotype. Mean±S.D.; ***p<0.001; Student's t-test. FIG. 6E shows the leakage of Sulfo-NHS-Biotin in the cerebellum of P10 Vtn^{RGE} mice. White boxes correspond to higher magnification panels showing tracer confinement to vessels (ICAM2) in wildtype mice and tracer leakage in Vtn^{RGE} mice. FIG. 6F shows the quantification of vessel permeability in wildtype and Vtn^{RGE} mice. n=4 animals per genotype. Mean±S.D.; ***p<0.001; Student's t-test. FIGS. 6G and 6H show EM images of HRP halting at tight junctions (arrows) in both retinas (FIG. 6G) and cerebellum (FIG. 6H) of wildtype and Vtn^{RGE} mice. Luminal (L) and abluminal sides indicated by dashed lines. FIGS. 6I and 6K show EM images of HRP-filled vesicles (arrowheads) in endothelial cells of retinas (FIG. 6I) and cerebellum (FIG. 6K) of wildtype and Vtn^{RGE} mice. FIGS. 6J and 6L show quantification of tracer-filled vesicles in endothelial cells of retinas (FIG. 6J) and cerebellum (FIG. 6L). n=4 animals per genotype, 15-20 vessels per animal. Mean±S.D.; ***p<0.001, **p<0.01; Student's t-test.

FIGS. 7A-7F show engagement of integrin α5 with vitronectin forms adhesion structures and actively inhibits endocytosis in primary brain endothelial cells. FIGS. 7A and 7B depict representative images (FIG. 7A) and quantification (FIG. 7B) of α5 containing adhesion structures in primary brain endothelial cells (phalloidin) grown on collagen IV, laminin and vitronectin-coated dishes. n=25 cells per condition from 3 independent experiments. Mean±S.D.; ***p<0.001; Student's t-test. FIGS. 7C and 7D show validation of two independent shRNAs targeting endogenous α5 in primary brain endothelial cells (phalloidin) and quantification of adhesion structures (FIG. 7D) in scramble vs Itga5 shRNAs. n=25 cells per condition from 3 independent experiments. Mean±S.D.; ***p<0.001, **p<0.01; one-way ANOVA with Tukey's post hoc test. FIG. 7E shows an endocytosis assay with membrane impermeable FM1-43FX in primary brain endothelial cells transfected with shRNAs targeting endogenous α5. FIG. 7F shows quantification of endocytic uptake of FM1-43FX. n=25 cells per condition from 3 independent experiments. Mean±S.D.; ***p<0.001, **p<0.01; one-way ANOVA with Tukey's post hoc test.

FIGS. 8A-8J show integrin α5 in endothelial cells is crucial for blood-CNS barrier function and vitronectin-integrin α5 mediated barrier regulation is independent of the caveolae pathway. FIG. 8A shows the in situ hybridization for Itga5, Pecam1 (endothelial gene) and Pdgfrb (pericyte gene) in P7 brain tissue. FIG. 8B shows a scatter plot of Itga5 and Itgav transcript numbers in pericytes versus endothelial cells from RNAscope in situ hybridization as shown in (FIG. 8A). Each dot represents an individual pericyte-endothelial cell pair, n=49 and 47 cell pairs respectively from 3 animals. FIG. 8C shows the depiction of tamoxifen injections in postnatal pups from P3-P5 and validation of tamoxifen-induced deletion of α5 from vessels (isolectin) in P10 mice. FIG. 8D shows Sulfo-NHS-Biotin (tracer) leaks out of vessels (isolectin) in P10 Cdh5:CreER+; Itga5^{fl/fl} mice. Tamoxifen administered P3-P5, see FIGS. 14A-14D. White boxes correspond to higher magnification images in (FIG. 8E). FIG. 8E shows tracer hotspots (arrowheads) in mice lacking endothelial Itga5. FIG. 8F shows the quantification of vessel permeability in wildtype and Cdh5:CreER+; Itga5^{fl/fl} mice. n=5 animals per genotype. Mean±S.D.; **p<0.01; Student's t-test. FIG. 8G shows Sulfo-NHS-Biotin leakage in cerebellum of P10 Cdh5:CreER+; Itga5^{fl/fl} mice. White boxes correspond to higher magnification images with tracer and vessels (ICAM2). FIG. 8H shows quantification of vessel permeability in wildtype and Cdh5:CreER+; Itga5^{fl/fl} mice. n=5 animals per genotype. Mean±S.D.; ***p<0.001; Student's t-test. FIG. 8I shows deletion of caveolin-1 did not rescue tracer leakage seen in Vtn^{-/-} mice. Sulfo-NHS-biotin leakage was observed in Vtn^{-/-}; Cav-1^{-/-} double knockouts, similar to Vtn^{-/-} single knockout mice. FIG. 8J shows quantification of vessel permeability in P10 retinas across all genotypes. n=4 animals per genotype. Mean±S.D.; ***p<0.001, n.s. not significant, p>0.05; one-way ANOVA with Tukey's post hoc test.

FIGS. 9A-9B show expression in CNS pericytes. FIG. 9A shows vitronectin detection using a vitronectin antibody in retinas of P7 Vtn^{-/-} mice. FIG. 9B depict in situ hybridization images showing Vtn, Pdgfrb (pericyte gene) and Pecam1 (endothelial gene) in cortex and cerebellum of P7 mouse.

FIGS. 10A-10C show that the leakage seen in retinas of Vtn^{-/-} mice is independent of tracer size and that leakage persists through adulthood. FIG. 10A shows co-injection of Sulfo-NHS-Biotin and 10 kDa Dextran shows leakage (arrowheads) of both tracers from vessels (isolectin) in P10 retinas of Vtn^{-/-} mice. FIG. 10B shows the quantification of 10 kDa Dextran leakage in wildtype and Vtn^{-/-} mice. n=5 animals per genotype. Mean±S.D.; ***p<0.001; Student's t-test. FIG. 10C depicts the leakage assay in 8-week old mice with Sulfo-NHS-Biotin (tracer) and vessels (isolectin). White boxes indicate the region shown in higher magnification images. Tracer-filled neuronal cell bodies (arrowheads) are indicated.

FIGS. 11A-11C show examples of vessels in cerebellum of Vtn^{-/-} mice with basement membranes completely filled with HRP. FIG. 11A shows EM Images of whole cross-sectional view of blood vessels in cerebellum of P10 mice. Lumen is filled with HRP. HRP-filled vesicles (arrowheads), pericytes (P) and nuclei outline (dashed lines) are indicated. FIG. 11B shows higher magnification images of boxed regions in FIG. 11A shows darker staining of basement membrane (black arrows) on the abluminal side (dashed line) of Vtn^{-/-} mice. FIG. 11C shows quantification of the percentage of vessels with basement membrane filled with HRP in wildtype and Vtn^{-/-} mice. n=4 animals per genotype. Mean±S.D.; ***p<0.001; Student's t-test.

FIGS. 12A-12I show that a lack of vitronectin does not alter vascular basement membrane composition. FIG. 12A depict tilescan images of collagen IV in P10 retinas of wildtype and Vtn^{-/-} mice. FIG. 12B shows higher magnification images of collagen IV, vessels (isolectin) and pericytes (NG2:DsRed). FIG. 12C display representative images of Collagen IV ensheathment of vessels (isolectin) and pericytes (NG2:DsRed) in P10 retinas. FIG. 12D and FIG. 12E are representative western blots (FIG. 12D) and quantification (FIG. 12E) of Fibronectin protein levels normalized to GAPDH in whole retinal lysates of P10 wildtype and Vtn^{-/-} mice. n=3 mice per genotype. Mean±S.D.; n.s. not significant, p>0.05; Student's t-test. FIG. 12F and 12G show representative images of perlecan (FIG. 12F) and laminin α4 (FIG. 12G) and vessels (isolectin) in P10 retinas of wildtype and Vtn^{-/-} mice. White boxes correspond to regions in the higher magnification images. FIG. 12H depict representative images of capillaries ensheathed by astrocyte endfeet. Cross-section of vessel and nucleus highlighted in dashed lines, pericytes (P), astrocyte endfeet (AE), L indicates vessel lumen filled with DAB. FIG. 12I shows quantification of the percent of capillary cross-section covered by astrocyte endfeet in cerebellum of P10 wildtype and Vtn^{-/-} mice. n=4 mice per genotype, 15-20 vessels per animal. Mean±S.D.; n.s. not significant, p>0.05; Student's t-test.

FIGS. 13A-13D show α5 containing adhesion structures are bonafide focal adhesions. FIGS. 13A and 13B show representative images (FIG. 13A) and quantification (FIG. 13B) of α5 adhesion structures in primary brain endothelial cells grown on collagen IV or laminin or vitronectin-coated dishes. n=15 to 25 cells per condition from 2 independent experiments. Mean±S.D.; n.s. not significant, p>0.05, ***p<0.001; one-way ANOVA with Tukey's post hoc test. FIG. 13C depicts representative images of α5 containing adhesion structures co-localizing with phospho-FAK (Y397) or paxillin or vinculin in endothelial cells (phalloidin). FIG. 13D shows the quantification of percentage of α5 adhesion structures positive for pFAK or paxillin or vinculin. n=15 to 20 cells per co-staining from 2 independent experiments. In each case, >90% of α5 containing adhesions are positive for these focal adhesion markers.

FIGS. 14A-14D show that endothelial-deletion of Itga5 does not result in impaired vascular patterning or morphology. FIG. 14A depicts tilescan images showing retinal vasculature of P10 retinas in mice lacking endothelial Itga5. FIGS. 14B-14D show the quantification of vessel density (FIG. 14B), capillary branching (FIG. 14C) and radial outgrowth (FIG. 14D) in retinas of wildtype and Cdh5:CreER+; Itga5^{fl/fl} mice. n=6 animals per genotype. Mean±S.D.; n.s. not significant, p>0.05; Student's t-test.

FIGS. 15A-15F show endothelial RGD-binding integrin, Itgav, is not required for CNS barrier integrity. FIG. 15A shows in situ hybridization for Itgav, Pecam1 (endothelial gene) and Pdgfrb (pericyte) in P7 brain tissue. See quantification in FIG. 6B. FIG. 15B shows Sulfo-NHS-Biotin (tracer) is confined to vessels (isolectin) in retinas of P10 Cdh5:CreER+; Itgav^{fl/fl} mice. White boxes correspond to higher magnification images in FIG. 15C. FIG. 15C shows tracer was confined to vessels with no leakage. FIG. 15D shows the quantification of vessel permeability in wildtype and Cdh5:CreER+; Itgav^{fl/fl} mice. n=5 animals per genotype. Mean±S.D.; n.s. not significant, p>0.05; Student's t-test. FIG. 15E shows Sulfo-NHS-Biotin is confined to vessels (ICAM2) in the cerebellum of P10 Cdh5:CreER+; Itgav^{fl/fl}. White boxes correspond to higher magnification images depicting tracer co-localizing with vessels (ICAM2). (FIG. 15F shows quantification of vessel permeability in wildtype and Cdh5:CreER+; Itgav^{fl/fl} mice. n=5 animals per genotype. Mean±S.D.; n.s. not significant, p>0.05; Student's t-test.

FIG. 16 shows that FAK inhibitor PF-562,271 decreases blood-CNS permeability in Vtn^{-/-} mice.

### DETAILED DESCRIPTION

The present disclosure, at least in part, provides compositions and uses thereof, kits and methods for regulating Blood-Central Nervous System (blood-CNS) barrier permeability (e.g., increasing or decreasing Blood-CNS barrier permeability) by regulating signaling between pericyte-derived vitronectin and integrin expressed on CNS endothelial cells (e.g., integrin α5). In some aspects, the present disclosure also provides a Blood-Central Nervous System (blood-CNS) barrier model comprising CNS endothelial cells and vitronectin or a plurality of cells secreting vitronectin, and methods for producing the same.

The blood-CNS barrier is a structure that separates circulating blood from the central nervous system (CNS). In some embodiments, the blood-CNS barrier includes the blood-brain barrier (BBB) and blood-retina barrier (BRB). The blood-CNS barrier lines the capillaries associated with the CNS and is comprised of endothelial cells and the tight junctions between them. The blood-brain barrier is formed by endothelial cells of the blood vessel (e.g., capillary) wall, astrocyte end-feet ensheathing the capillary, and pericytes embedded in the blood vessel (e.g., capillary) basement membrane. In some embodiments, the endothelial cells of the CNS are brain endothelial cells. In some embodiments, the brain endothelial cells are arterial endothelial cells, venous endothelial cells, and/or capillary endothelial cells. In some embodiments, the brain capillary endothelial cells are brain microvascular endothelial cells (BMVECs). In some embodiments, the endothelial cells of the CNS are spinal cord endothelial cells. In some embodiments, the endothelial cells of the CNS are retina vasculature endothelial cells. In some embodiments, the retina vasculature endothelial cells are superficial plexus arterial endothelial cells, superficial plexus venous endothelial cells, intermediate plexus endothelial cells, or deep plexus endothelial cells.

The blood-CNS barrier generally excludes large hydrophilic molecules and pathogens (e.g., bacteria, viruses, or parasites) from entering the CNS while allowing the passage of small hydrophobic molecules, such as lipids and oxygen. Other molecules are actively transported across the blood-CNS barrier, e.g., glucose. The restrictive permeability of CNS endothelial cells that constitute these barriers is a result of specialized tight junctions and low rates of transcytosis, which limit substance exchange between blood and CNS tissue. While the blood-CNS barrier is generally very effective at excluding, e.g., pathogens, from the CNS, the blood-CNS barrier poses a formidable obstacle when a drug needs to be delivered to the CNS. For example, antibodies and most antibiotics will not cross the blood-CNS barrier. The degradation of the blood-CNS barrier is a feature of many neurodegenerative diseases, e.g., multiple sclerosis. Accordingly, methods and compositions for modulating the permeability of the blood-CNS barrier, both by increasing or decreasing the permeability of the barrier, have a role in the treatment of a wide variety of diseases that impact the CNS. (See, e.g., Kadry et al., A blood-brain barrier overview on structure, function, impairment, and biomarkers of integrity, Fluids and Barriers of the CNS, volume 17, Article number: 69 (2020).

The present disclosure, at least in part, is based on the theory that the barrier properties of the CNS endothelial cells are not intrinsic to those cells. Rather, the CNS endothelial cells require active induction and maintenance from the CNS environment to induce and maintain the barrier properties (see, e.g., P. A. Stewart et al., Developing nervous tissue induces formation of blood-brain barrier characteristics in invading endothelial cells: A study using quail-chick transplantation chimeras. Dev. Biol. 84, 183-192 (1981)). In some embodiments, pericytes and astrocytes are required for maintaining blood-CNS barrier integrity. The pericyte is a perivascular cell type that encapsulates the microvasculature of the brain and spinal cord. Pericytes play a crucial role in the development and maintenance of the blood-CNS barrier and have a multitude of important functions in the brain. Pericyte impairment has been implicated in various neurovascular pathology (Bennett et al., Pericytes Across the Lifetime in the Central Nervous System, Front. Cell. Neurosci., 12 Mar. 2021). In some aspects, the present disclosure is based on the discovery that an extracellular matrix (ECM) protein secreted by the pericytes is capable of maintaining blood-CNS barrier integrity via an integrin expressed by CNS endothelial cells. In some embodiments, the ECM protein secreted by pericytes is vitronectin. In some embodiments, the integrin expressed by CNS endothelial cells is an integrin (e.g., integrin α5). In some embodiments, the pericyte are Pdgfrb expressing pericytes. In some embodiments, vitronectin regulates barrier function via binding to its integrin receptors on endothelial cells in the CNS. In some embodiments, vitronectin regulates barrier function via binding to integrin α5 on endothelial cells in the CNS (e.g., Ayloo et al., Pericyte-to-endothelial cell signaling via vitronectin-integrin regulates blood-CNS barrier, Neuron 110, 1-15, May 18, 2022). In some embodiments, binding of vitronectin to integrin α5 maintains blood-CNS barrier integrity or decreases blood-CNS barrier permeability by inhibiting transcytosis in CNS endothelial cells.

Vitronectin is a multifunctional glycoprotein of 75 kD that binds to various biological ligands and plays a key role in tissue remodeling by regulating cell adhesion through binding to different types of integrins, mainly via the RGD sequence. In addition to being an ECM protein, vitronectin is also an abundant protein circulating in the plasma which has been shown to mediate the complement pathway and play a role in tissue repair and wound healing (Leavesley et al., Vitronectin—master controller or micromanager? IUBMB Life 65, 807-818, 2013; Preissner and Reuning, Vitronectin in vascular context: Facets of a multitalented matricellular protein. Semin. Thromb. Hemost. 37, 408-424.). The circulating vitronectin is produced by the liver. In some embodiments, the circulating vitronectin produced by the liver does not affect the blood-CNS barrier integrity.

Integrins are transmembrane adhesion receptors expressed on the surface of most mammalian cell types. Integrin subunits function as heterodimers consisting of one of 18 α- and one of the eight β-subunits which dictate ligand specificity (Hynes. Integrins: bidirectional, allosteric signaling machines. Cell. 2002; 110:673-687). The most well-studied integrin classes in endothelial cells engage ECM components by directly binding ECM proteins. In addition to functioning as important adhesion receptors, integrins are bi-directional signaling hubs involved in numerous fundamental cellular processes including cell migration, survival, and proliferation. On one hand, integrin affinity is regulated through so-called “inside-out” integrin signaling (integrin activation) whereby extracellular signals are transduced into the cell through cell-surface receptors (e.g., receptor tyrosine kinases, G-protein-coupled receptors) that in turn ultimately lead to the binding of cytoplasmic, integrin activating proteins such as talin and kindlin to the β-integrin cytoplasmic tail (Shattil S J, Kim C, Ginsberg M H. The final steps of integrin activation: the end game. Nat Rev Mol Cell Biol. 2010; 11:288-300). On the other hand, integrin “outside-in” signaling occurs in response to integrin binding extracellular ligands and subsequent activation of cytoplasmic signaling pathways important for the maturation of adhesion complexes (Calderwood et al., Integrins and actin filaments: reciprocal regulation of cell adhesion and signaling. J Biol Chem. 2000; 275:22607-22610.). For example, outside-in integrin signaling leads to the recruitment of actin-binding proteins (e.g., talin, vinculin, α-actinin, tensin, filamin) and adaptor proteins that either directly or indirectly link integrin to the actin cytoskeleton. Accordingly, integrins serve as a hub for signaling pathways essential to diverse and fundamental cellular functions. Integrin adhesion promotes the activation of focal adhesion kinase (FAK), a non-receptor tyrosine kinase that also functions as an important signaling hub required for FA maturation and disassembly during cell migration.

In some aspects, the present disclosure provides compositions and methods for increasing Blood-Central Nervous System (Blood-CNS) barrier permeability to treat a disease (e.g., brain and CNS tumor, ALS, etc) in a subject, the method comprising administering to the subject an inhibitor (e.g., inhibitory nucleic acids, small molecule inhibitors, and/or antibodies targeting vitronectin and/or integrin) of vitronectin-integrin signaling at the Blood-CNS Barrier.

Other aspects of the present disclosure relate to co-delivering of a molecule (e.g., a therapeutic agent) with an inhibitor of the vitronectin-integrin signaling at the Blood-CNS Barrier to facilitate the CNS delivery of the molecule (e.g., a therapeutic agent).

In some aspects, the present disclosure provides a method for decreasing Blood-Central Nervous System (blood-CNS) barrier permeability for treating a disease in a subject, the method comprising administering to the subject an agent that promotes vitronectin-integrin signaling in the central nervous system (CNS).

### I. Increasing Blood-Central Nervous System (Blood-CNS) Barrier Permeability

In some aspects, the present disclosure provides methods for increasing blood-CNS barrier permeability. In some embodiments, the method comprises administering to a subject an inhibitor of vitronectin-integrin signaling. In some embodiments, the vitronectin is secreted by CNS pericytes. In some embodiments, the integrin (e.g., integrin α5) is expressed on the cell surface of CNS endothelial cells (e.g., CNS capillary endothelial cells).

In some embodiments, the inhibitor of vitronectin-integrin signaling is a vitronectin inhibitor. In some embodiments, inhibition of vitronectin secreted by CNS pericytes results in increased blood-CNS barrier permeability. In some embodiments, inhibition of circulating vitronectin does not result in increased blood-CNS barrier permeability.

In some embodiments, the vitronectin inhibitor is capable of inhibiting vitronectin expression and/or activity. In some embodiments, the vitronectin inhibitor is an inhibitory nucleic acid targeting vitronectin mRNA. As described herein, an inhibitory nucleic acid, refers to nucleic acids capable of inhibiting expression or activity of a target gene (e.g., DNA, RNA, or protein of the target gene), for example, VTN. Non-limiting examples of inhibitory nucleic acids include e.g., dsRNA, siRNA, shRNA, miRNA, amiRNA, antisense oligonucleotides (ASOs), DNA or RNA aptamers, etc. An ASO is a small chain of nucleotides, generally 18-30 nucleotides long, that targets messenger RNA (mRNA) and is capable of altering mRNA expression through a variety of mechanisms, including ribonuclease H mediated decay of the pre-mRNA, direct steric blockage, and exon content modulation through splicing site binding on pre-mRNA. A small interfering RNA (siRNA), also known as short interfering RNA or silencing RNA, is a double-stranded non-coding RNA molecules, typically 20-27 base pairs in length, that interferes with the expression of specific genes with complementary nucleotide sequences by degrading mRNA after transcription, preventing translation. A short hairpin RNA or small hairpin RNA (shRNA) is an artificial RNA molecules with a tight hairpin turn that can be used to silence target gene expression via RNA interference (RNAi). A microRNA (abbreviated miRNA) is a small single-stranded non-coding RNA molecule (containing about 22 nucleotides) that functions in RNA silencing and post-transcriptional regulation of gene expression via base-pairing with complementary sequences within mRNA molecules. An amiRNA is an artificial miRNA. A mixmer is an oligomer consisting of alternating short stretches of LNA and DNA. An LNA, or Locked Nucleic Acid, also known as bridged nucleic acid (BNA), or inaccessible RNA, is a modified RNA nucleotide in which the ribose moiety is modified with an extra bridge connecting the 2′ oxygen and 4′ carbon.

In some embodiments, an inhibitory nucleic acid targeting VTN is an siRNA. siRNA molecules comprise a specific antisense sequence in addition to the reverse complement (sense) sequence.

The specificity of siRNA molecules may be determined by the binding of the antisense strand of the molecule to its target RNA (e.g., VTN mRNA). In some embodiments, the siRNA molecules are 60, 65, 70, 75, 80, 85, 90, 95, 100 or more base pairs in length. In some embodiments, the antisense sequence of the siRNA molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the antisense sequence of the siRNA molecules are 8 to 30 base pairs in length, 10 to 15 base pairs in length, 10 to 20 base pairs in length, 15 to 25 base pairs in length, 19 to 21 base pairs in length, or 21 to 23 base pairs in length.

Following selection of an appropriate target RNA sequence, siRNA molecules that comprise a nucleotide sequence complementary to all or a portion of the target sequence, i.e., an antisense sequence, can be designed and prepared using methods known in the art.

In some embodiments, the antisense sequence of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, the sense sequence of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, siRNA molecules comprise an antisense sequence comprising a region of complementarity to a target region in a VTN mRNA (e.g., human VTN mRNA or mouse VTN mRNA). In some embodiments, the region of complementarity is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a target region in a VTN mRNA (e.g., human VTN mRNA or mouse VTN mRNA). In some embodiments, the target region is a region of consecutive nucleotides in a VTN mRNA (e.g., human VTN mRNA or mouse VTN mRNA). In some embodiments, a complementary nucleotide sequence need not be 100% complementary to that of its target to be specifically hybridizable or specific for a VTN mRNA (e.g., human VTN mRNA or mouse VTN mRNA). Exemplary human VTN mRNA and mouse VTN mRNA sequence are set forth in SEQ ID NOs: 1 and 2.

In some embodiments, siRNA molecules comprise an antisense sequence that comprises a region of complementarity to in a VTN mRNA (e.g., human VTN mRNA or mouse VTN mRNA) sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a VTN mRNA (e.g., human VTN mRNA or mouse VTN mRNA). In some embodiments, the region of complementarity comprises a nucleotide sequence that contains no more than 1, 2, 3, 4, or 5 base mismatches compared to the complementary portion of a VTN mRNA (e.g., human VTN mRNA or mouse VTN mRNA). In some embodiments, the region of complementarity comprises a nucleotide sequence that has up to 3 mismatches over 15 bases, up to 2 mismatches over 10 bases, or up to 1 mismatch over 5 bases.

In some embodiments, siRNA molecules targeting VTN comprise an antisense strand which comprises a nucleotide sequence that is at least 60%, at least 65%, at least 70%, at least 75%, at least 80%, at least 85%, at least 90%, at least 95%, or 100% identical to the sequence as set forth in SEQ ID NOs: 7 or 8. In some embodiments, siRNA molecules targeting VTN comprise an antisense strand at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in SEQ ID NOs: 7 or 8.

In some embodiments, siRNA molecules targeting VTN comprise a sense strand which comprises a nucleotide sequence that is at least 60%, at least 65%, at least 70%, at least 75%, at least 80%, at least 85%, at least 90%, at least 95%, or 100% identical to the sequence as set forth in SEQ ID NOs: 9 or 10. In some embodiments, siRNA molecules targeting VTN comprise a sense strand at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in SEQ ID NOs: 9 or 10. Exemplary siRNAs targeting VTN are set forth below:

```
siRNA#1 targeting VTN:
Sense strand 5′-GUCUAAGCGUAGAAGCCGATT-3′(SEQ ID NO: 9)
                                            |||||||||||||||||||
Antisense strand 3′-TTCAGAUUCGCAUCUUCGGCU-5′(SEQ ID NO: 7)
siRNA#2 targeting VTN:
Sense strand 5′-CUACAACAACUAUGAUUAUTT-3′(SEQ ID NO: 10)
                                            |||||||||||||||||||
Antisense strand 3′-TGGAUGUUGUUGAUACUAAUA-5′(SEQ ID NO: 8)
```

In some embodiments, the present disclosure also contemplates delivering inhibitory nucleic acid targeting VTN mRNA that are known in the art, for example, has-miR-335-5p, has-miR-26b-59, #4457308 siRNA IDs s76001, and s76002 from Ambion, or #sc-36821, #sc-36820 and #sc-270256 from Santa Cruz.

In some embodiments, the vitronectin inhibitor is an antibody, an antibody variant or an antigen-binding fragment thereof targeting vitronectin. An antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) NIH publication no. 91-3242. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules, Mol. Immunol. 31:1047-1058). Antibodies, antibody variants and antigen-binding fragments targeting vitronectin have been previously described, see, e.g., US-2007-0048325, LS-B10245 from LSBio, 130-00001-20 from RayBiotech, 10424-R102, 10424-R103, 50585-R019 from SinoBiological.

In some embodiments, inhibiting the integrin side of vitronectin-integrin signaling increases blood-CNS barrier permeability. In some embodiments, an inhibitor of vitronectin-integrin signaling is an integrin inhibitor. In some embodiments, an integrin inhibitor is an integrin α5 inhibitor. Integrin α5 is a integrin that is encoded by the ITGA5 gene. Integrin α5 joins with beta 1 to form a fibronectin receptor. In some embodiments, an integrin α5 inhibitor is capable of inhibiting integrin α5 expression and/or activity. In some embodiments, the integrin α5 inhibitor is an inhibitory nucleic acid targeting ITGA5 (e.g., ITGA5 mRNA). As described herein, an inhibitory nucleic acid, refers to nucleic acids capable of inhibiting expression or activity of a target gene (e.g., DNA, RNA, or protein of the target gene), for example, ITGA5. In some embodiments, an inhibitory nucleic acids targeting ITGA5 includes dsRNA, siRNA, shRNA, miRNA, amiRNA, antisense oligonucleotides (ASOs), or DNA or RNA aptamers targeting ITGA5.

In some embodiments, an inhibitory nucleic acid targeting ITGA5 is an shRNA. shRNA molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in the addition or removal of one, two, three, or more nucleotides from the 3′ end and/or (e.g., and) the 5′ end of either or both strands). A spacer can be of a sufficient length to permit the antisense and sense sequences to anneal and form a double-stranded structure (or stem) prior to cleavage of the spacer (and, optionally, subsequent processing steps that may result in the addition or removal of one, two, three, four, or more nucleotides from the 3′ end and/or (e.g., and) the 5′ end of either or both strands). A spacer sequence may be an unrelated nucleotide sequence that is situated between two complementary nucleotide sequence regions which, when annealed into a double-stranded nucleic acid, comprise a shRNA.

The specificity of shRNA molecules may be determined by the binding of the antisense strand of the molecule to its target RNA (e.g., ITGA5 mRNA). In some embodiments, the shRNA molecules are 60, 65, 70, 75, 80, 85, 90, 95, 100 or more base pairs in length. In some embodiments, the antisense sequence of the shRNA molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the antisense sequence of the shRNA molecules are 8 to 30 base pairs in length, 10 to 15 base pairs in length, 10 to 20 base pairs in length, 15 to 25 base pairs in length, 19 to 21 base pairs in length, or 21 to 23 base pairs in length.

Following selection of an appropriate target RNA sequence, shRNA molecules that comprise a nucleotide sequence complementary to all or a portion of the target sequence, i.e., an antisense sequence, can be designed and prepared using methods known in the art (see, e.g., Moore et al., Short Hairpin RNA (shRNA): Design, Delivery, and Assessment of Gene Knockdown, Methods Mol Biol. 2010; 629: 141-158).

The shRNA molecules can comprise a hairpin (i.e., when two regions of the same strand, usually complementary in nucleotide sequence when read in opposite directions, base-pair to form a double helix that ends in an unpaired loop), or asymmetric hairpin (i.e. hairpin with a strand overhang) secondary structure, having self-complementary sense and antisense strands. In some embodiments, the shRNA targeting inhibitory nucleic acid described herein comprises an antisense sequence and a sense sequence.

In some embodiments, the antisense sequence of the shRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, the sense sequence of the shRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense sequence is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, shRNA molecules comprise an antisense sequence comprising a region of complementarity to a target region in a ITGA5 mRNA (e.g., human ITGA5 mRNA or mouse ITGA5 mRNA). In some embodiments, the region of complementarity is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a target region in a ITGA5 mRNA (e.g., human ITGA5 mRNA or mouse ITGA5 mRNA). In some embodiments, the target region is a region of consecutive nucleotides in a ITGA5 mRNA (e.g., human ITGA5 mRNA or mouse ITGA5 mRNA). In some embodiments, a complementary nucleotide sequence need not be 100% complementary to that of its target to be specifically hybridizable or specific for a ITGA5 mRNA (e.g., human ITGA5 mRNA or mouse ITGA5 mRNA).

In some embodiments, shRNA molecules comprise an antisense sequence that comprises a region of complementarity to in a ITGA5 mRNA (e.g., human ITGA5 mRNA or mouse ITGA5 mRNA) sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a ITGA5 mRNA (e.g., human ITGA5 mRNA or mouse ITGA5 mRNA). In some embodiments, the region of complementarity comprises a nucleotide sequence that contains no more than 1, 2, 3, 4, or 5 base mismatches compared to the complementary portion of a ITGA5 mRNA (e.g., human ITGA5 mRNA or mouse ITGA5 mRNA). In some embodiments, the region of complementarity comprises a nucleotide sequence that has up to 3 mismatches over 15 bases, up to 2 mismatches over 10 bases, or up to 1 mismatch over 5 bases.

In some embodiments, shRNA molecules targeting ITGA5 comprise a nucleotide sequence that is at least 60%, at least 65%, at least 70%, at least 75%, at least 80%, at least 85%, at least 90%, at least 95%, or 100% identical to the sequence as set forth in SEQ ID NOs: 3 or 4. In some embodiments, shRNA molecules targeting ITGA5 comprise at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in SEQ ID NOs: 1 or 2.

shRNA#1 targeting ITGA5:
(SEQ ID NO: 3)
CCCAGCAGGGAGTCGTATTTACTCGAGTAAATACGACTCCCTGCTGGG
shRNA#2 targeting ITGA5:
(SEQ ID NO: 4)
ATCAACTTGGAACCATAATTACTCGAGTAATTATGGTTCCAAGTTGAT

In some embodiments, the integrin α5 inhibitor is an antibody targeting integrin α5. Antibodies targeting integrin α5 include, but are not limited to, Volociximab (Mould et al., Defining the topology of integrin alpha5beta1-fibronectin interactions using inhibitory anti-alpha5 and anti-beta1 monoclonal antibodies. J Biol Chem. 1997 Jul. 11; 272(28):17283-92); anti-integrin α5 antibody as described in WO 2009/100110, WO 2010/111254, and WO 2007/134876; EPR7854, ab112183, ab239400, and ab221606 from Abcam; or PA5-82027, PA5-25433, MA5-15568, 702178, and 711210 from Invitrogen.

In some embodiments, the inhibitory nucleic acids targeting vitronectin or integrin a5 can be administered to the subject using any suitable known method, for example, but not limited to, direct injection, viral vector mediated delivery (e.g., AAV, retrovirus, adeno virus, or lentivirus), or ceDNA.

In some embodiments, an integrin α5 inhibitor is a peptide containing RGD. In some embodiments, an RGD peptide inhibits the binding of integrin α5 on CNS endothelial cells to the ECM proteins (e.g., vitronectin) at the blood-CNS barrier. Examples of RGD peptide include, but are not limited to, Echistatin, Cyclic RGD peptides, or RGD peptides described by RGD peptides described by Kapp et al., A Comprehensive Evaluation of the Activity and Selectivity Profile of Ligands for RGD-binding Integrins, Scientific Reports volume 7, Article number: 39805 (2017).

In some embodiments, the integrin α5 inhibitor is a small molecule inhibitor targeting integrin α5. In some embodiments, the small molecule integrin α5 inhibitor is a non-peptidic RGD memetic. Non-limiting examples of non-peptidic RGD memetic include Ac-PHSCN-NH2 (Stoeltzing, O. et al. Inhibition of integrin alpha5beta1 function with a small peptide (ATN-161). Int. J. Cancer 104, 496-503 (2003)); JSM6427 (Stragies, R. et al. Design and synthesis of a new class of selective integrin alpha5beta1 antagonists. J. Med. Chem. 50, 3786-3794 (2007)); 44b (Heckmann, D. et al. Rational Design of Highly Active and Selective Ligands for the α5β1 Integrin Receptor. ChemBioChem 9, 1397-1407 (2008)); and sn243 (Neubauer, S. et al. Pharmacophoric Modifications Lead to Superpotent αvβ3 Integrin Ligands with Suppressed α5β1 Activity; J. Med. Chem. 57, 3410-3417 (2014).

In some embodiments, administration of an effective amount of any one of the inhibitors described herein or a combination thereof of vitronectin-integrin signaling at the blood-CNS barrier results in increased blood-CNS barrier permeability.

An effective amount of an inhibitor described herein may be an amount sufficient to have a therapeutic benefit in a subject, e.g., to extend the lifespan of a subject, to improve and/or reverse in the subject one or more symptoms of disease, or to slow disease progression. The effective amount will depend on a variety of factors such as, for example, the species, age, weight, health of the subject, and the tissue to be targeted, and may thus vary among subject and tissue. An effective amount may also depend on the inhibitor used.

In some embodiments, administration of the inhibitors described herein may result in inhibition of vitronectin-integrin signaling of one or more of the foregoing endothelial cells. In some embodiments, administration of the inhibitors described herein may result in inhibition of vitronectin-integrin signaling in all of the foregoing endothelial cells.

An effective amount may also depend on the mode of administration. For example, targeting endothelial cells in the CNS by intravenous administration or subcutaneous injection may require different (e.g., higher or lower) doses, in some cases, than targeting an endothelial cells in the CNS by another method (e.g., local injection to the CNS). In some embodiments, the inhibitor described herein is a small molecule and can be taken orally.

In some embodiments, administering an inhibitor of vitronectin-integrin signaling at the Blood-CNS Barrier decreases the level and/or activity of vitronectin and/or integrin α5. As used herein, administration of the inhibitor described herein decreases the expression and/or activity of vitronectin and/or integrin α5 by at least 10% or more, e.g., by 10% or more, 50% or more, 100% or more, 200% or more, 500% or more, or 1000% or more. In some embodiments, administration of the inhibitors described herein results in increasing of the permeability of the blood-CNS barrier in the subject. In some embodiments, administration of the inhibitor described herein results in increasing the permeability of the blood-CNS barrier in the subject by at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 99%, at least 100%, at least 150%, at least 200%, at least 300%, at least 400%, at least 500% or more compared the permeability of the blood-CNS barrier of the subject prior to administration of the inhibitor. The permeability of blood-CNS barrier can be measured using any suitable technique or method known in the art, e.g., neuroimaging techniques including dynamic perfusion CT (PCT) and dynamic contrast-enhanced magnetic resonance imaging (DCEMRI), quantification of protein biomarkers (e.g., neuron-specific enolase (NSE), glial fibrillary acidic protein (GFAP), and S100β) in the cerebral spinal fluid (CSF), etc.

In some embodiments, the methods described herein relate to treating a subject having or diagnosed as having a disease affecting the CNS, e.g., a neurological disease or a condition treated by delivering therapeutic agents to the CNS. Subjects having a disease affecting the CNS can be identified by a physician using current methods of diagnosing such conditions. Symptoms and/or complications of such conditions which characterize these conditions and aid in diagnosis are known in the art and include, but are not limited to, loss of neural function (e.g. lack of coordination, lack of sensation, altered behaviors, inflammation of the CNS, headaches, etc). Tests that may aid in a diagnosis of such conditions can include, but are not limited to, CT scan, MRI scan, spinal tap, brain biopsy, nerve biopsy, electroencephalogram (EEG), lumbar puncture, physical examination, nerve conduction studies, and/or blood tests. For some conditions, a family history of the condition (e.g., by genetic analysis), or exposure to risk factors for the condition can also aid in determining if a subject is likely to have the condition or in making a diagnosis.

In some embodiments, the inhibitors described herein or a composition thereof can be administered to a subject having or diagnosed as having a disease affecting the CNS. In some embodiments, the methods described herein comprise administering an effective amount of an inhibitor described herein or a composition thereof, to a subject in order to alleviate a symptom of a disease affecting the CNS. As used herein, “alleviating a symptom” is ameliorating any condition or symptom associated with the disease affecting the CNS. As compared with an equivalent untreated control, such reduction is by at least 5%, 10%, 20%, 40%, 50%, 60%, 80%, 90%, 95%, 99% or more as measured by any standard technique.

In some embodiments, an inhibitor of vitronectin-integrin signaling at the blood-CNS barrier can be administered to a subject in need thereof in combination with a therapeutic agent to the central nervous system. In some embodiments, the administration of an inhibitor of vitronectin-integrin signaling at the blood-CNS barrier decreases blood-CNS barrier permeability thereby facilitating the delivery of the therapeutic agent to the CNS. The therapeutic agent can be any agent for the treatment of any disease, provided that it is desired that the therapeutic agent reaches the central nervous system. In some embodiments, methods which comprise administering an inhibitor of vitronectin-integrin signaling at the blood-CNS barrier can further comprise administering a therapeutic agent to the subject. Non-limiting examples of such therapeutic agents can include, antibiotics, antibodies, anticonvulsant (e.g., gabapentin), chemotherapeutics, anti-inflammatories, neurotransmitters, pain medication (e.g., morphine), peptides, nucleic acids (e.g. RNAi-based therapies), or psychiatric drugs. In some embodiments, a subject in need of increased permeability of the blood-brain barrier is in need of treatment for a condition selected from the group consisting of neuromuscular diseases (e.g., Amyotrophic Lateral Sclerosis (ALS), Ataxia, Cerebral Palsy, Muscular Dystrophy), neurodegenerative disease (e.g., Alzheimer's Disease), brain and nerve tumors (e.g., Chordomas, Craniopharyngiomas, Gangliocytomas, Glomus jugulare, Meningiomas, Pineocytomas, Pituitary adenomas, Schwannomas, Gliomas, Astrocytomas, Ependymomas, Glioblastoma multiforme (GBM), Medulloblastomas, Oligodendrogliomas, Hemangioblastomas, Rhabdoid tumors, medulloblastomas, low-grade astrocytomas (pilocytic), ependymomas, craniopharyngiomas and brainstem gliomas, etc.), Neurogenetic Diseases (e.g., Ataxia including spinocerebellar ataxias, olivopontocerebellar atrophies, and multiple system degeneration, CADASIL (Cerebral Autosomal Dominant Arteriopathy with Sub-cortical Infarcts and Leukoencephalopathy), Charcot-Marie-Tooth disease (hereditary neuropathy), Cognitive disorders (e.g., familial Alzheimer's disease and other familial dementias including frontotemporal dementia, familial Pick's disease, familial Creutzfeldt-Jakob disease, Familial amyotrophic lateral sclerosis (familial ALS also known as Lou Gehrig's disease), Familial dystonia including Dopa-responsive dystonia, Fragile X and Fragile X associated Tremor Ataxia Syndrome (FXTAS), Hereditary Spastic Paraplegia, Huntington's disease, Leukodystrophy including adrenomyeloneuropathy and cerebrotendinous xanthomatosis, Lysosomal storage disorders including Gaucher, Niemann-Pick, and Fabry diseases, Mitochondrial encephalomyopathies (MELAS syndrome), Mucopolysaccharidoses, Neurofibromatosis, Primary Lateral Sclerosis, Tourette's syndrome, Tuberous sclerosis, Von-Hippel-Lindau disease, Wilsons disease), or neuropsychiatric disorders (e.g., schizophrenia (SZ), bipolar disorder (BD), major depressive disorder (MDD) and attention deficit hyperactivity disorder (ADHD), etc). The identity of such CNS therapeutic agents are known in the art and described, e.g. in Ghose et al. J Comb Chem 1999 1:55-68 and Pardridge. NeuroRx 2005 2:3-14; each of which is incorporated by reference herein in its entirety. In some embodiments, a central nervous system therapeutic agent can inhibit the activity and/or expression of a therapeutic target gene associated with a central nervous system disease (e.g. examples of such genes are described below herein), e.g., it can be an inhibitory nucleic acid or an inhibitory antibody reagent.

In some embodiments, the central nervous system therapeutic agent is less than about 1000 kDa in size. In some embodiments, the central nervous system therapeutic agent is less than about 500 kDa in size. In some embodiments, the central nervous system therapeutic reagent is less than about 300 kDa in size. In some embodiments, the central nervous system therapeutic reagent is less than about 200 kDa in size. In some embodiments, the central nervous system therapeutic reagent is less than about 70 kDa in size. In some embodiments, the central nervous system therapeutic reagent can be, e.g., a biologic agent (e.g., an enzyme, an antibody, a polypeptide), a sugar, and/or a small molecule.

In some embodiments, the therapeutic agent is an agent that does not normally cross the blood-CNS barrier. In some embodiments, the CNS therapeutic agent is an agent that inefficiently crosses the blood-CNS barrier, e.g. a therapeutically effective dose of the agent is unable to cross the blood-CNS barrier when administered systemically. In some embodiments, the CNS therapeutic agent is an agent that does efficiently cross the blood-CNS barrier, e.g., a therapeutically effective dose of the agent is able to cross the blood-CNS barrier when administered systemically. Administration of an inhibitor of vitronectin-integrin signaling at the blood-CNS barrier can increase the permeability of the blood-CNS barrier such that, e.g., a therapeutically effective dose of the CNS therapeutic agent is able to reach the CNS or the necessary dose of the CNS therapeutic agent is lowered.

Delivery of an inhibitor of vitronectin-integrin signaling at the blood-CNS barrier to a mammalian subject (e.g., human) may be by, for example, injection to the CNS. In some embodiments, the injection is direct injection to the CNS (e.g., intracerebral injection, intraventricular injection, intracisternal injection, intraparenchymal injection, intrathecal injection, and any combination of the foregoing). In some embodiments, the injection is systemic injection (e.g., intravenous injection, intradermal injection, or subcutaneous injection). In some embodiments, the inhibitor can be administered orally.

### II. Decreasing Blood-CNS Barrier Permeability

In some aspects, the present disclosure also provides methods for decreasing blood-CNS barrier permeability (i.e., maintaining blood-CNS integrity) in a subject. In some embodiments, the method comprises administering to the subject an agent that promotes vitronectin-integrin signaling at the blood-CNS barrier. In some embodiments, an agent that promotes the vitronectin-integrin signaling increases expression and/or activity of vitronectin and/or integrin α5.

In some embodiments, an agent that promotes the vitronectin-integrin signaling at the blood-CNS barrier is a recombinant vitronectin or a fragment thereof. In some embodiments, an agent that promotes the vitronectin-integrin signaling at the blood-CNS barrier is a recombinant human vitronectin or a fragment thereof. In some embodiments, the recombinant human vitronectin comprises an amino acid sequence An exemplary human vitronectin amino acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the amino acid sequence as set forth in SEQ ID NO: 11 (NP_000629.3). In some embodiments, an exemplary human vitronectin amino acid sequence is set forth in SEQ ID NO: 11 (NP_000629.3):

MAPLRPLLILALLAWVALADQESCKGRCTEGFNVDKKCQC
DELCSYYQSCCTDYTAECKPQVTRGDVFTMPEDEYTVYDD
GEEKNNATVHEQVGGPSLTSDLQAQSKGNPEQTPVLKPEE
EAPAPEVGASKPEGIDSRPETLHPGRPQPPAEEELCSGKP
FDAFTDLKNGSLFAFRGQYCYELDEKAVRPGYPKLIRDVW
GIEGPIDAAFTRINCQGKTYLFKGSQYWRFEDGVLDPDYP
RNISDGFDGIPDNVDAALALPAHSYSGRERVYFFKGKQYW
EYQFQHQPSQEECEGSSLSAVFEHFAMMQRDSWEDIFELL
FWGRTSAGTRQPQFISRDWHGVPGQVDAAMAGRIYISGMA
PRPSLAKKQRFRHRNRKGYRSQRGHSRGRNQNSRRPSRAT
WLSLFSSEESNLGANNYDDYRMDWLVPATCEPIQSVFFFS
GDKYYRVNLRTRRVDTVDPPYPRSIAQYWLGCPAPGHL

In some embodiments, an agent that promotes the vitronectin-integrin signaling at the blood-CNS barrier is a recombinant mouse vitronectin or a fragment thereof. In some embodiments, the recombinant mouse vitronectin comprises an amino acid sequence An exemplary mouse vitronectin amino acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the amino acid sequence as set forth in SEQ ID NO: 12 (AAA40558.1). In some embodiments, an exemplary mouse vitronectin amino acid sequence is set forth in SEQ ID NO: 12 (AAA40558.1):

MAPLRPFFILALVAWVSLADQESCKGRCTQGFMASKKCQC
DELCTYYQSCCADYMEQCKPQVTRGDVFTMPEDDYWSYDY
VEEPKNNTNTGVQPENTSPPGDLNPRTDGTLKPTAFLDPE
EQPSTPAPKVEQQEEILRPDTTDQGTPEFPEEELCSGKPF
DAFTDLKNGSLFAFRGQYCYELDETAVRPGYPKLIQDVWG
IEGPIDAAFTRINCQGKTYLFKGSQYWRFEDGVLDPGYPR
NISEGFSGIPDNVDAAFALPAHRYSGRERVYFFKGKQYWE
YEFQQQPSQEECEGSSLSAVFEHFALLQRDSWENIFELLF
WGRSSDGAREPQFISRNWHGVPGKVDAAMAGRIYVTGSLS
HSAQAKKQKSKRRSRKRYRSRRGHRRSQSSNSRRSSRSIW
FSLFSSEESGLGTKNNYDYDMDWLVPATCEPIQSVYFFSG
DKYYRVNLRTRRVDSVNPPYPRSIAQYWLGCPTSEK

In some embodiments, an agent that promotes the vitronectin-integrin signaling at the blood-CNS barrier is a nucleic acid encoding vitronectin. In some embodiments, the nucleic acid encoding vitronectin encodes a mammalian vitronectin (e.g., human vitronectin, mouse vitronectin, rat vitronectin, non-human primate vitronectin, etc.). In some embodiments, the nucleic acid encoding vitronectin encodes a human vitronectin. In some embodiments, the nucleic acid encoding vitronectin encodes a mouse vitronectin.

Human vitronectin mRNA sequence is set forth in SEQ ID NO: 1 (NM_000638). In some embodiments, the nucleic acid encoding human vitronectin comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 1. An exemplary nucleotide sequence encoding human vitronectin protein is set forth in SEQ ID NO: 1 (NM_000638):

CTCCTTCCCTGTCTCTGCCTCTCCCTCCCTTCCTCAGGCA
TCAGAGCGGAGACTTCAGGGAGACCAGAGCCCAGCTTGCC
AGGCACTGAGCTAGAAGCCCTGCCATGGCACCCCTGAGAC
CCCTTCTCATACTGGCCCTGCTGGCATGGGTTGCTCTGGC
TGACCAAGAGTCATGCAAGGGCCGCTGCACTGAGGGCTTC
AACGTGGACAAGAAGTGCCAGTGTGACGAGCTCTGCTCTT
ACTACCAGAGCTGCTGCACAGACTATACGGCTGAGTGCAA
GCCCCAAGTGACTCGCGGGGATGTGTTCACTATGCCGGAG
GATGAGTACACGGTCTATGACGATGGCGAGGAGAAAAACA
ATGCCACTGTCCATGAACAGGTGGGGGGCCCCTCCCTGAC
CTCTGACCTCCAGGCCCAGTCCAAAGGGAATCCTGAGCAG
ACACCTGTTCTGAAACCTGAGGAAGAGGCCCCTGCGCCTG
AGGTGGGCGCCTCTAAGCCTGAGGGGATAGACTCAAGGCC
TGAGACCCTTCATCCAGGGAGACCTCAGCCCCCAGCAGAG
GAGGAGCTGTGCAGTGGGAAGCCCTTCGACGCCTTCACCG
ACCTCAAGAACGGTTCCCTCTTTGCCTTCCGAGGGCAGTA
CTGCTATGAACTGGACGAAAAGGCAGTGAGGCCTGGGTAC
CCCAAGCTCATCCGAGATGTCTGGGGCATCGAGGGCCCCA
TCGATGCCGCCTTCACCCGCATCAACTGTCAGGGGAAGAC
CTACCTCTTCAAGGGTAGTCAGTACTGGCGCTTTGAGGAT
GGTGTCCTGGACCCTGATTACCCCCGAAATATCTCTGACG
GCTTCGATGGCATCCCGGACAACGTGGATGCAGCCTTGGC
CCTCCCTGCCCATAGCTACAGTGGCCGGGAGCGGGTCTAC
TTCTTCAAGGGGAAACAGTACTGGGAGTACCAGTTCCAGC
ACCAGCCCAGTCAGGAGGAGTGTGAAGGCAGCTCCCTGTC
GGCTGTGTTTGAACACTTTGCCATGATGCAGCGGGACAGC
TGGGAGGACATCTTCGAGCTTCTCTTCTGGGGCAGAACCT
CTGCTGGTACCAGACAGCCCCAGTTCATTAGCCGGGACTG
GCACGGTGTGCCAGGGCAAGTGGACGCAGCCATGGCTGGC
CGCATCTACATCTCAGGCATGGCACCCCGCCCCTCCTTGG
CCAAGAAACAAAGGTTTAGGCATCGCAACCGCAAAGGCTA
CCGTTCACAACGAGGCCACAGCCGTGGCCGCAACCAGAAC
TCCCGCCGGCCATCCCGCGCCACGTGGCTGTCCTTGTTCT
CCAGTGAGGAGAGCAACTTGGGAGCCAACAACTATGATGA
CTACAGGATGGACTGGCTTGTGCCTGCCACCTGTGAACCC
ATCCAGAGTGTCTTCTTCTTCTCTGGAGACAAGTACTACC
GAGTCAATCTTCGCACACGGCGAGTGGACACTGTGGACCC
TCCCTACCCACGCTCCATCGCTCAGTACTGGCTGGGCTGC
CCAGCTCCTGGCCATCTGTAGGAGTCAGAGCCCACATGGC
CGGGCCCTCTGTAGCTCCCTCCTCCCATCTCCTTCCCCCA
GCCCAATAAAGGTCCCTTAGCCCCGA

Mouse vitronectin mRNA sequence is set forth in SEQ ID NO: 2 (MUSVITA). In some embodiments, the nucleic acid encoding mouse vitronectin comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 2. An exemplary nucleotide sequence encoding mouse vitronectin protein is set forth in SEQ ID NO: 2 (MUSVITA):

AGAGTCATGCAAGGGCCGCTGCACTCAGGGTTTCATGGCC
AGCAAGAAGTGTCAGTGTGACGAGCTTTGCACTTACTATC
AGAGCTGCTGTGCCGACTACATGGAGCAGTGCAAGCCCCA
AGTAACGCGGGGGGACGTGTTCACTATGCCAGAGGATGAT
TATTGGAGCTATGACTACGTGGAGGAGCCCAAGAACAATA
CCAACACCGGTGTGCAACCCGAGAACACCTCTCCACCCGG
TGACCTAAATCCTCGGACGGACGGCACTCTAAAGCCGACA
GCCTTCCTAGATCCTGAGGAACAGCCAAGCACCCCAGCGC
CTAAAGTGGAGCAACAGGAGGAGATCCTAAGGCCCGACAC
CACTGATCAAGGGACCCCTGAGTTTCCAGAGGAAGAACTG
TGCAGTGGAAAGCCCTTTGACGCCTTCACGGATCTCAAGA
ATGGGTCCCTCTTTGCCTTCCGAGGGCAGTACTGCTATGA
GCTAGATGAGACGGCAGTGAGGCCTGGGTACCCCAAACTT
ATCCAAGATGTCTGGGGCATTGAGGGCCCCATCGATGCTG
CCTTCACTCGCATCAACTGTCAGGGGAAGACCTACTTGTT
CAAGGGTAGTCAGTACTGGCGCTTTGAGGATGGGGTCCTG
GACCCTGGTTATCCCCGAAACATCTCCGAAGGCTTCAGTG
GCATACCAGACAATGTTGATGCAGCGTTCGCCCTTCCTGC
CCACCGTTACAGTGGCCGGGAAAGGGTCTACTTCTTCAAG
GGGAAGCAGTACTGGGAGTACGAATTTCAGCAGCAACCCA
GCCAGGAGGAGTGCGAAGGCAGCTCTCTGTCAGCCGTGTT
TGAGCACTTTGCCTTGCTTCAGCGGGACAGCTGGGAGAAC
ATTTTCGAACTCCTCTTCTGGGGCAGATCCTCTGATGGAG
CCAGAGAACCCCAATTCATCAGCCGGAACTGGCATGGTGT
GCCAGGGAAAGTGGACGCTGCTATGGCCGGCCGCATCTAC
GTCACTGGCTCCTTATCCCACTCTGCCCAAGCCAAAAAAC
AGAAGTCTAAGCGTAGAAGCCGAAAGCGCTATCGTTCACG
CCGAGGCCACAGACGCAGCCAGAGCTCGAACTCCCGTCGT
TCATCACGTTCAATCTGGTTCTCTTTGTTCTCCAGCGAGG
AGAGTGGGCTAGGAACCAAAAACAACTATGATTATGATAT
GGACTGGCTTGTACCTGCCACCTGCGAGCCCATTCAGAGC
GTCTATTTCTTCTCTGGAGACAAATACTACCGAGTCAACC
TTAGAACCCGGCGAGTGGACTCTGTGAATCCTCCCTACCC
ACGCTCCATTGCTCAGTATTGGCTGGGCTGCCCGACCTCT
GAGAAGTAGGAATCAGAGCCCACTCGGCTGAGCTTCAGGA
GCCTCATCTCTTTCTCCCAGCCCAATAAAAAGTCTGTTGG
CTACGAAAAA

In some embodiments, an agent that promotes vitronectin-integrin signaling at the blood-CNS barrier is a nucleic acid encoding integrin α5. In some embodiments, the nucleic acid encoding integrin α5 encodes a mammalian vitronectin (e.g., human vitronectin, mouse vitronectin, rat vitronectin, non-human primate vitronectin, etc.). In some embodiments, the nucleic acid encoding vitronectin encodes a human integrin α5. In some embodiments, the nucleic acid encoding vitronectin encodes a mouse integrin α5.

Human integrin α5 mRNA sequence is set forth in SEQ ID NO: 5 (NM_002205). In some embodiments, the nucleic acid encoding human integrin α5 comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 5. An exemplary nucleotide sequence encoding human integrin α5 protein is set forth in SEQ ID NO: 5 (NM_002205):

ATTCGCCTCTGGGAGGTTTAGGAAGCGGCTCCGGGTCGGT
GGCCCCAGGACAGGGAAGAGCGGGCGCTATGGGGAGCCGG
ACGCCAGAGTCCCCTCTCCACGCCGTGCAGCTGCGCTGGG
GCCCCCGGCGCCGACCCCCGCTGCTGCCGCTGCTGTTGCT
GCTGCTGCCGCCGCCACCCAGGGTCGGGGGCTTCAACTTA
GACGCGGAGGCCCCAGCAGTACTCTCGGGGCCCCCGGGCT
CCTTCTTCGGATTCTCAGTGGAGTTTTACCGGCCGGGAAC
AGACGGGGTCAGTGTGCTGGTGGGAGCACCCAAGGCTAAT
ACCAGCCAGCCAGGAGTGCTGCAGGGTGGTGCTGTCTACC
TCTGTCCTTGGGGTGCCAGCCCCACACAGTGCACCCCCAT
TGAATTTGACAGCAAAGGCTCTCGGCTCCTGGAGTCCTCA
CTGTCCAGCTCAGAGGGAGAGGAGCCTGTGGAGTACAAGT
CCTTGCAGTGGTTCGGGGCAACAGTTCGAGCCCATGGCTC
CTCCATCTTGGCATGCGCTCCACTGTACAGCTGGCGCACA
GAGAAGGAGCCACTGAGCGACCCCGTGGGCACCTGCTACC
TCTCCACAGATAACTTCACCCGAATTCTGGAGTATGCACC
CTGCCGCTCAGATTTCAGCTGGGCAGCAGGACAGGGTTAC
TGCCAAGGAGGCTTCAGTGCCGAGTTCACCAAGACTGGCC
GTGTGGTTTTAGGTGGACCAGGAAGCTATTTCTGGCAAGG
CCAGATCCTGTCTGCCACTCAGGAGCAGATTGCAGAATCT
TATTACCCCGAGTACCTGATCAACCTGGTTCAGGGGCAGC
TGCAGACTCGCCAGGCCAGTTCCATCTATGATGACAGCTA
CCTAGGATACTCTGTGGCTGTTGGTGAATTCAGTGGTGAT
GACACAGAAGACTTTGTTGCTGGTGTGCCCAAAGGGAACC
TCACTTACGGCTATGTCACCATCCTTAATGGCTCAGACAT
TCGATCCCTCTACAACTTCTCAGGGGAACAGATGGCCTCC
TACTTTGGCTATGCAGTGGCCGCCACAGACGTCAATGGGG
ACGGGCTGGATGACTTGCTGGTGGGGGCACCCCTGCTCAT
GGATCGGACCCCTGACGGGCGGCCTCAGGAGGTGGGCAGG
GTCTACGTCTACCTGCAGCACCCAGCCGGCATAGAGCCCA
CGCCCACCCTTACCCTCACTGGCCATGATGAGTTTGGCCG
ATTTGGCAGCTCCTTGACCCCCCTGGGGGACCTGGACCAG
GATGGCTACAATGATGTGGCCAGGAGGGCTGGGCTCTAAG
CCTTCCCAGGTTCTGCAGCCCCTGTGGGCAGCCAGCCACA
CCCCAGACTTCTTTGGCTCTGCCCTTCGAGGAGGCCGAGA
CCTGGATGGCAATGGATATCCTGATCTGATTGTGGGGTCC
TTTGGTGTGGACAAGGCTGTGGTATACAGGGGCCGCCCCA
TCGTGTCCGCTAGTGCCTCCCTCACCATCTTCCCCGCCAT
GTTCAACCCAGAGGAGCGGAGCTGCAGCTTAGAGGGGAAC
CCTGTGGCCTGCATCAACCTTAGCTTCTGCCTCAATGCTT
CTGGAAAACACGTTGCTGACTCCATTGGTTTCACAGTGGA
ACTTCAGCTGGACTGGCAGAAGCAGAAGGGAGGGGTACGG
CGGGCACTGTTCCTGGCCTCCAGGCAGGCAACCCTGACCC
AGACCCTGCTCATCCAGAATGGGGCTCGAGAGGATTGCAG
AGAGATGAAGATCTACCTCAGGAACGAGTCAGAATTTCGA
GACAAACTCTCGCCGATTCACATCGCTCTCAACTTCTCCT
TGGACCCCCAAGCCCCAGTGGACAGCCACGGCCTCAGGCC
AGCCCTACATTATCAGAGCAAGAGCCGGATAGAGGACAAG
GCTCAGATCTTGCTGGACTGTGGAGAAGACAACATCTGTG
TGCCTGACCTGCAGCTGGAAGTGTTTGGGGAGCAGAACCA
TGTGTACCTGGGTGACAAGAATGCCCTGAACCTCACTTTC
CATGCCCAGAATGTGGGTGAGGGTGGCGCCTATGAGGCTG
AGCTTCGGGTCACCGCCCCTCCAGAGGCTGAGTACTCAGG
ACTCGTCAGACACCCAGGGAACTTCTCCAGCCTGAGCTGT
GACTACTTTGCCGTGAACCAGAGCCGCCTGCTGGTGTGTG
ACCTGGGCAACCCCATGAAGGCAGGAGCCAGTCTGTGGGG
TGGCCTTCGGTTTACAGTCCCTCATCTCCGGGACACTAAG
AAAACCATCCAGTTTGACTTCCAGATCCTCAGCAAGAATC
TCAACAACTCGCAAAGCGACGTGGTTTCCTTTCGGCTCTC
CGTGGAGGCTCAGGCCCAGGTCACCCTGAACGGTGTCTCC
AAGCCTGAGGCAGTGCTATTCCCAGTAAGCGACTGGCATC
CCCGAGACCAGCCTCAGAAGGAGGAGGACCTGGGACCTGC
TGTCCACCATGTCTATGAGCTCATCAACCAAGGCCCCAGC
TCCATTAGCCAGGGTGTGCTGGAACTCAGCTGTCCCCAGG
CTCTGGAAGGTCAGCAGCTCCTATATGTGACCAGAGTTAC
GGGACTCAACTGCACCACCAATCACCCCATTAACCCAAAG
GGCCTGGAGTTGGATCCCGAGGGTTCCCTGCACCACCAGC
AAAAACGGGAAGCTCCAAGCCGCAGCTCTGCTTCCTCGGG
ACCTCAGATCCTGAAATGCCCGGAGGCTGAGTGTTTCAGG
CTGCGCTGTGAGCTCGGGCCCCTGCACCAACAAGAGAGCC
AAAGTCTGCAGTTGCATTTCCGAGTCTGGGCCAAGACTTT
CTTGCAGCGGGAGCACCAGCCATTTAGCCTGCAGTGTGAG
GCTGTGTACAAAGCCCTGAAGATGCCCTACCGAATCCTGC
CTCGGCAGCTGCCCCAAAAAGAGCGTCAGGTGGCCACAGC
TGTGCAATGGACCAAGGCAGAAGGCAGCTATGGCGTCCCA
CTGTGGATCATCATCCTAGCCATCCTGTTTGGCCTCCTGC
TCCTAGGTCTACTCATCTACATCCTCTACAAGCTTGGATT
CTTCAAACGCTCCCTCCCATATGGCACCGCCATGGAAAAA
GCTCAGCTCAAGCCTCCAGCCACCTCTGATGCCTGAGTCC
TCCCAATTTCAGACTCCCATTCCTGAAGAACCAGTCCCCC
CACCCTCATTCTACTGAAAAGGAGGGGTCTGGGTACTTCT
TGAAGGTGCTGACGGCCAGGGAGAAGCTCCTCTCCCCAGC
CCAGAGACATACTTGAAGGGCCAGAGCCAGGGGGGTGAGG
AGCTGGGGATCCCTCCCCCCCATGCACTGTGAAGGACCCT
TGTTTACACATACCCTCTTCATGGATGGGGGAACTCAGAT
CCAGGGACAGAGGCCCCAGCCTCCCTGAAGCCTTTGCATT
TTGGAGAGTTTCCTGAAACAACTTGGAAAGATAACTAGGA
AATCCATTCACAGTTCTTTGGGCCAGACATGCCACAAGGA
CTTCCTGTCCAGCTCCAACCTGCAAAGATCTGTCCTCAGC
CTTGCCAGAGATCCAAAAGAAGCCCCCAGCTAAGAACCTG
GAACTTGGGGAGTTAAGACCTGGCAGCTCTGGACAGCCCC
ACCCTGGTGGGCCAACAAAGAACACTAACTATGCATGGTG
CCCCAGGACCAGCTCAGGACAGATGCCACACAAGGATAGA
TGCTGGCCCAGGGCCCAGAGCCCAGCTCCAAGGGGAATCA
GAACTCAAATGGGGCCAGATCCAGCCTGGGGTCTGGAGTT
GATCTGGAACCCAGACTCAGACATTGGCACCTAATCCAGG
CAGATCCAGGACTATATTTGGGCCTGCTCCAGACCTGATC
CTGGAGGCCCAGTTCACCCTGATTTAGGAGAAGCCAGGAA
TTTCCCAGGACCCTGAAGGGGCCATGATGGCAACAGATCT
GGAACCTCAGCCTGGCCAGACACAGGCCCTCCCTGTTCCC
CAGAGAAAGGGGAGCCCACTGTCCTGGGCCTGCAGAATTT
GGGTTCTGCCTGCCAGCTGCACTGATGCTGCCCCTCATCT
CTCTGCCCAACCCTTCCCTCACCTTGGCACCAGACACCCA
GGACTTATTTAAACTCTGTTGCAAGTGCAATAAATCTGAC
CCAGTGCCCCCACTGACCAGAACTAGAA

Mouse integrin α5 mRNA sequence is set forth in SEQ ID NO: 6 (NM_010577). In some embodiments, the nucleic acid encoding mouse integrin α5 comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% identical to the nucleotide sequence of SEQ ID NO: 6. An exemplary nucleotide sequence encoding mouse integrin α5 protein is set forth in SEQ ID NO: 6 (NM_010577):

AGTCTGAGCCAGGCCAGACCACCGGCCTCCAGCTGGGGCC
GGAGCCAGGGTTCAGAGGTGCGGCCCCCCACGCCCCTTCG
GGTGCGGGGCGCGGCCTCGGCGTCCCCGGGACCCAGGAAT
GCCCCCCCGCCAGCCGGCCCGGCGGCCGGGGGGAGGGCCC
AGCCGGGAGTTTGGCAAACTCCTCCCCGCGTTGAGTCATT
CGCCTCTGGGAGGTTTAGGAAGCGGCTCCGGGTCGGTGGC
CCCAGGACAGGGAAGAGCGGGCACTATGGGGAGCTGGACG
CCACGGTCGCCTCGATCTCCTCTCCACGCGGTGCTGCTGC
GCTGGGGGCCCCGACGCCTACCGCCGCTGCTGCCTCTGCT
GCTGCTACTGTGGCCGCCACCACTCCAGGTTGGGGGCTTC
AACCTAGACGCGGAGGCCCCGGCGGTGCTCTCCGGGCCCC
CCGGCTCCCTTTTTGGCTTCTCCGTGGAGTTTTACCGGCC
GGGAAGGGACGGAGTCAGTGTGCTGGTTGGGGCACCCAAG
GCTAACACTAGCCAGCCAGGTGTACTGCAAGGTGGTGCTG
TCTATGTGTGTCCCTGGGGCACCAGTCCTATCCAGTGCAC
CACCATTCAATTTGACAGCAAAGGCTCCCGGATTCTGGAG
TCCTCACTGTACAGTGCCAAGGGAGAGGAGCCTGTGGAGT
ACAAGTCCTTGCAGTGGTTCGGAGCAACAGTTCGGGCCCA
TGGCTCCTCCATCTTGGCATGTGCTCCACTGTATAGCTGG
CGCACAGAAAAGGACCCACAGAATGACCCAGTGGGCACCT
GCTACCTCTCCACAGAAAACTTCACCCGGATTCTGGAGTA
CGCACCTTGCCGCTCAGATTTTGGCAGTGCAGCAGGGCAG
GGCTACTGCCAAGGGGGCTTCAGTGCTGAGTTCACCAAGA
CTGGCCGTGTGGTCCTGGGTGGACCTGGAAGCTACTTCTG
GCAAGGCCAGATCCTGTCCGCCACTCAAGAGCAGATCTCG
GAGTCCTATTACCCAGAGTATCTCATCAACCCTGTTCAGG
GGCAGCTGCAGACCCGCCAGGCCAGCTCCGTCTATGATGA
CAGCTACTTGGGATACTCTGTGGCTGTGGGTGAATTCAGT
GGTGATGACACAGAAGACTTTGTTGCTGGCGTGCCCAAGG
GGAACCTCACCTATGGCTATGTCACCGTCCTTAATGGCTC
AGACATCCACTCCCTCTACAACGTCTCAGGAGAACAGATG
GCCTCCTACTTCGGCTATGCTGTGGCTGCCACTGATACCA
ATGGAGATGGGCTAGATGACCTACTGGTAGGGGCACCCCT
GCTCATGGAGCGGACAGCTGATGGGAGACCTCAGGAGGTG
GGCAGGGTCTACATCTATCTGCAGCGCCCAGCGGGCATAG
ATCCCACACCCACCCTCACCCTCACTGGGCAAGATGAGTT
CAGCCGATTCGGCAGCTCCTTGACACCCTTGGGGGACCTG
GACCAAGACGGCTACAATGATGTCGCCATTGGGGCTCCAT
TTGGTGGGGAGGCCCAGCAGGGAGTCGTATTTATATTCCC
GGGAGGCCCAGGAGGACTGAGCACTAAACCTTCCCAGGTT
TTGCAGCCTCTTTGGGCAGCTGGCCGTACCCCAGACTTCT
TTGGCTCTGCCCTTCGAGGAGGACGAGATCTGGATGGCAA
TGGATACCCTGATCTAATCGTTGGATCCTTTGGTGTGGAC
AAGGCTCTGGTGTACAGAGGGCGGCCCATCATATCTGCCA
GCGCATCTCTCACCATCTTCCCCTCCATGTTCAACCCAGA
GGAGCGCAGTTGCAGCCTGGAAGGGAACCCTGTGTCCTGC
ATCAACCTTAGCTTCTGCCTCAATGCCTCTGGAAAACATG
TCCCCAACTCTATAGGCTTCGAGGTGGAACTCCAACTGGA
CTGGCAGAAGCAAAAGGGAGGGGTCCGGCGGGCACTGTTC
CTGACTTCCAAGCAGGCCACCCTTACCCAGACCCTGCTTA
TCCAGAATGGGGCTCGGGAGGACTGCAGGGAGATGAAGAT
CTACCTCAGGAATGAATCAGAATTCAGAGACAAACTCTCC
CCAATTCACATTGCCCTCAACTTCTCCTTGGACCCCAAAG
CTCCCATGGACAGCCATGGCCTCCGGCCAGTTCTACACTA
CCAAAGCAAAAGCAGGATAGAGGACAAGGCCCAGATCTTG
CTGGACTGTGGTGAAGACAATATCTGTGTGCCTGACCTGC
AGCTGGATGTGTATGGGGAGAAGAAACATGTGTACCTGGG
TGACAAGAACGCACTGAACCTGACATTCCATGCCCAAAAT
CTGGGTGAGGGCGGTGCCTATGAAGCCGAGCTTCGGGTCA
CAGCCCCTCTAGAGGCCGAGTACTCAGGACTTGTCAGACA
CCCAGGGAACTTCTCCAGCCTGAGCTGTGACTACTTTGCT
GTGAACCAGAGCCGCCAGCTGGTGTGTGACCTGGGCAACC
CCATGAAGGCAGGCACCAGTCTCTGGGGTGGCCTTCGGTT
CACTGTTCCTCATCTTCAAGACACAAAGAAAACCATCCAG
TTTGACTTTCAGATCCTCAGCAAGAACCTGAACAACTCAC
AAAGCAACGTGGTCTCCTTCCCACTCTCGGTGGAGGCTCA
AGCCCAGGTCTCCCTTAATGGTGTCTCCAAGCCTGAAGCT
GTGATTTTCCCAGTCAGCGACTGGAATCCTCAAGACCAGC
CTCAGAAGGAGGAAGACTTGGGCCCAGCTGTCCACCATGT
CTACGAGCTCATCAACCAGGGGCCCAGCTCCATCAGCCAG
GGTGTGCTGGAGCTCAGCTGTCCACAGGCTCTGGAAGGCC
AACAGCTCCTCTATGTGACCAAGGTGACAGGACTCAGCAA
CTGCACCTCCAACTACACCCCCAACTCACAGGGCCTGGAG
TTGGATCCAGAGACCTCTCCACACCACCTGCAGAAACGAG
AGGCTCCAGGGAGGAGTTCTACTGCCTCAGGAACACAAGT
TCTGAAATGCCCTGAAGCCAAGTGTTTCAGGCTGCGCTGT
GAGTTTGGGCCACTGCACCGCCAAGAGAGCCGTAGTCTGC
AGCTGCATTTCCGAGTCTGGGCCAAGACCTTCTTGCAGCG
GGAATACCAGCCATTTAGCCTTCAGTGTGAGGCTGTATAT
GAAGCTCTGAAGATGCCCTACCAGATCCTGCCTCGGCAGC
TTCCCCAAAAGAAACTTCAGGTGGCCACAGCCGTGCAGTG
GACCAAGGCAGAAGGCAGCAATGGTGTCCCGTTGTGGATC
ATCATCCTAGCCATTCTTTTTGGCCTCCTGCTCCTAGGTC
TGCTCATCTACGTCCTCTACAAGCTCGGCTTCTTCAAACG
TTCCCTCCCCTACGGCACAGCCATGGAAAAAGCTCAGCTC
AAGCCTCCAGCCACCTCAGATGCCTGAGCCCTCCTGATCT
CAGACTCATGATCCCGAAGAGCCAGTCCAATACCTTCCGA
CAGGGAGGGACTAGGCACTTCCTGAAGGTGCTGAGGGCTG
GGGAGAAGCTCTTCTTCCTAGCCCAGAGACATACTTGAAG
GGCCAGAGCTGGGGGGCAAGAAGCTTGTGGATCCTTCCCC
CCATGCACTGTGAAGGACCCTCGTTTACACATGCCCTCTC
GTGGATGGGGGCCTCAGGTCCAGGGACAGAAGCTCCAGCT
TTCCTTCAGCCTTTGCATTTTGGAGACTTTTCTGAAATCA
TCCCATCTGACTCCGGCCTGCAAAGATGCATCTTTGGTCT
TGCCAGAGAACCAAAGGAAGTCTGCAGCTAAGAGCCTGAG
ACTTGGGGAGTGAGACCTGGCAACTCCAACAGCCCCCATC
CTGGAAGGCCAATGAAGAACACTAACCGTGGATGGTGCTG
CAGGACCAGCTCAGGACAAATCCCAAACAAAGGTCGATGC
TGGCCCAGACCCAGATCCATGGAAGTCAGAAAGAGAATGG
AGCCAGACCCAGTCTGGGGCCTAGGGTTGATCTGGAGCCC
AGTCTCGGATTCTAAGAACCTATGCTAGCAGACCCAAGGT
GGCATTTGGGTCAGCTGCAGACCTGGGCTTAGAAACCTAT
TTATCCCTGACTTAGGACAAGTCAGGAATTGCCCAGGACC
CTAACCAGGCCACATTGGCAAACAGACCTGGAACCTCAGC
CTGGCCACTCATTCCCCAGAGAGAACGGGAAGCCCCAGGC
CTGTATGACCTGGGTTCTGCCCACCAGCTGCACTGACGCT
GCCCCTCTCTCCCTAGCCAACCCTCCCCTCTCCTCCAGAA
CCCACCCCAACTTATTTAAACTCTGTTGCAAGTGCAATAA
ACCCCACCCACTGCCCCCACTGACCAAAAAAAAAAAAAAA
AAAAAAAAAAAAAAAAAA

In some embodiments, the nucleic acids encoding a vitronectin or integrin a5 can be administered to the subject using any suitable known method, for example, but not limited to, direct injection, viral vector mediated delivery (e.g., AAV, retrovirus, adeno virus, or lentivirus), or ceDNA.

The present disclosure, at least in part, is based on the surprising discovery that inhibition of focal adhesion kinase (FAK) in CNS endothelial cells results in a decrease in blood-CNS barrier permeability (i.e., maintaining blood-CNS integrity). In some aspects, the method comprises administering to the subject an focal adhesion kinase (FAK) inhibitor. Focal adhesion kinase (FAK) and proline-rich tyrosine kinase 2 (Pyk2) are closely related nonreceptor protein tyrosine kinases. FAK can regulate cell proliferation, survival, and motility, and plays an essential role in development. In some embodiments, the FAK inhibitor is PF-562271 (Slack-Davis et al., Cellular characterization of a clinical candidate FAK family kinase inhibitor, PF-562,271, Cancer Res (2007) 67: 1642; Roberts et al., Antitumor Activity and Pharmacology of a Selective Focal Adhesion Kinase Inhibitor, PF-562,271, Cancer Res (2008) 68 (6): 1935-1944). Other non-limiting examples of FAK inhibitors include FAK inhibitors described in international PCT application publications, WO 2012/110774, WO 2012/110773, WO 2012/022408, WO 2020/135442, and WO 2018/148666.

Without wishing to be bound by any particular theory, efficient delivery of an agent that promotes vitronectin-integrin signaling at the blood-CNS barrier or a FAK inhibitor (e.g., PF-562,271) may be useful for the treatment of a subject having a disease associated with endothelial cell dysfunction (e.g., head trauma, stroke, etc). In certain embodiments, the endothelial cells are endothelial cells in the CNS. Accordingly, methods and compositions for treating diseases associated with endothelial cell dysfunction are also provided herein. In some aspects, the disclosure provides a method for treating a disease associated with endothelial cell dysfunction, the method comprising: administering to a subject having or suspected of having a disease associated with endothelial cell dysfunction an effective amount of an agent that promotes vitronectin-integrin signaling at the blood-CNS barrier or a FAK inhibitor (e.g., PF-562,271).

Endothelial cells (e.g., endothelial cells in the CNS) can be healthy endothelial cells (e.g., endothelial cells in the CNS not having a dysfunction, or at risk of developing endothelial cell dysfunction), or dysfunctional endothelial cells (e.g., endothelial cells causing abnormal vasculature permeability, hemodynamics, or neuroimmune crosstalk, etc). As used herein, “or at risk of developing endothelial cell dysfunction” refers to a subject having an increased probability of developing endothelial cell dysfunction than the general population due to the presence of a risk factor. Exemplary categories of risk factors for developing endothelial cell dysfunction include, but are not limited to, genetics, head trauma, vascular disease, prior brain surgery, disease, age, race, and family history (e.g., positive family history of vascular disease, high cholesterol, high blood pressure, or diabetes).

As used herein, an “disease associated with endothelial cell dysfunction” is a disease or condition that results from the dysfunction of endothelial cells (e.g., endothelial cells in the CNS). In some embodiments, a disease associated with CNS endothelial cell dysfunction includes but is not limited to, retinal disease (e.g., diabetic retinopathy), neurodegenerative disease (e.g., Huntington's disease, dementia), acute injury of the CNS (e.g., stroke and head trauma), Neuroinfectious disease (e.g., encephalitis, sepsis, COVID-19), primary and metastatic cancers of the CNS, autoimmune disease of the CNS (e.g., multiple sclerosis), and other neuroinflammatory conditions. In some embodiments, the disease associated with CNS endothelial cell dysfunction is a CNS primary cancer, such as glioblastoma, meningioma, or lymphoma. In some embodiments, the disease associated with CNS endothelial cell dysfunction is a metastatic cancers to the brain such as metastatic lung cancer, metastatic breast cancer, or melanoma. In some embodiments, the disease associated with CNS endothelial cell dysfunction is a neuroinflammatory disease, such as CNS Lupus, CNS Lyme Disease, Neurosarcoidosis, Neuromyelitis optica (NMO), or Paraneoplastic and Autoimmune Encephalitis. In some embodiments, the disease associated with CNS endothelial cell dysfunction is a dementia and/or cognitive disorder, such as dementia resulting from Alzheimer's disease, Lewy body dementia, frontotemporal dementia, encephalopathy, or post-acute COVID syndrome.

In some embodiments, an agent that promotes vitronectin-integrin signaling at the blood-CNS barrier or a FAK inhibitor (e.g., PF-562,271) can be administered to a subject in need of improved integrity (e.g., decreased permeability) of tight junctions of the blood-CNS barrier. In some embodiments, the subject in need of improved quality of tight junctions of the blood-CNS barrier can be a subject who has been diagnosed with or determined to have abnormally high permeability of the blood-brain barrier, e.g., repeated infections of the CNS, or in which abnormal levels of a systemically administered tracer molecule reach the CNS. In some embodiments, the subject having abnormally high blood-CNS barrier permeability described herein has an increasing of the permeability of the blood-CNS barrier compared to a healthy subject. In some embodiments, the subject having abnormally high blood-CNS barrier permeability described herein has an increasing of the permeability of the blood-CNS barrier by at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 99%, at least 100%, at least 150%, at least 200%, at least 300%, at least 400%, at least 500% or more compared the permeability of the blood-CNS barrier of a healthy subject. The permeability of blood-CNS barrier can be measured using any suitable technique or method known in the art, e.g., neuroimaging techniques including dynamic perfusion CT (PCT) and dynamic contrast-enhanced magnetic resonance imaging (DCEMRI), quantification of protein biomarkers (e.g., neuron-specific enolase (NSE), glial fibrillary acidic protein (GFAP), and S100β) in the cerebral spinal fluid (CSF), etc.

In some embodiments, the subject in need of improved quality of tight junctions of the blood-brain barrier can be a subject in need of treatment (e.g. having, diagnosed as having, or at risk of developing) a condition selected from the group consisting of dementia, encephalitis, sepsis, COVID-19, primary and metastatic cancers of the CNS, autoimmune disease of the CNS (e.g., multiple sclerosis), and other neuroinflammatory conditions. In some embodiments, the disease is a CNS primary cancer, such as glioblastoma, meningioma, or lymphoma. In some embodiments, the disease is a metastatic cancers to the brain such as metastatic lung cancer, metastatic breast cancer, or melanoma. In some embodiments, the disease is a neuroinflammatory disease, such as CNS Lupus, CNS Lyme Disease, Neurosarcoidosis, Neuromyelitis optica (NMO), or Paraneoplastic and Autoimmune Encephalitis. In some embodiments, the disease is a dementia and/or cognitive disorder, such as dementia resulting from Alzheimer's disease, Lewy body dementia, frontotemporal dementia, encephalopathy, or post-acute COVID syndrome.

In some embodiments, administration of an agent that promotes vitronectin-integrin signaling at the blood-CNS barrier or a FAK inhibitor (e.g., PF-562,271) can slow or halt the progression of a neurodegenerative disease. In some embodiments, administration of an agent that promotes the vitronectin-integrin signaling at the blood-CNS barrier or a FAK inhibitor (e.g., PF-562,271) can slow or prevent the development of at least some signs or symptoms of any of the diseases described herein.

Delivery of an agent that promotes vitronectin-integrin signaling at the blood-CNS barrier or a FAK inhibitor (e.g., PF-562,271) to a mammalian subject may be by, for example, injection to the CNS. In some embodiments, the injection is direct injection to the CNS (e.g., intracerebral injection, intraventricular injection, intracisternal injection, intraparenchymal injection, intrathecal injection, and any combination of the foregoing). In some embodiments, the injection is systemic injection (e.g., intravenous injection, intradermal injection, or subcutaneous injection). In some embodiments, an agent that promotes vitronectin-integrin signaling at the blood-CNS barrier or a FAK inhibitor (e.g., PF-562,271) can be administered orally.

### III. Blood-CNS Model

In some aspects, the present disclosure is based on the discovery that vitronectin is capable of inducing and maintaining barrier properties of CNS endothelial cells. Accordingly, the present disclosure provides a blood-CNS model. In some embodiments, the blood-CNS model comprises a plurality of endothelial cells and vitronectin. In some embodiments, the blood-CNS model comprises a plurality of endothelial cells and a plurality of cells secreting vitronectin. In some embodiments, the plurality of cells secreting vitronectin are pericytes.

In some embodiments, a Blood-CNS model comprises a permeable support having a surface comprising vitronectin; and a plurality of endothelial cells (e.g., CNS endothelial cells), wherein the plurality of CNS endothelial cells expresses claudin-5, occludin, ZO-1 and GLUT-1 WNT7A, WNT7B, or ZO-3, and the hBECs are grown to confluence on the permeable support. The blood vessels in the brain form a blood-CNS barrier, which limits the flow of molecules and ions from the blood into neural tissues. The blood-CNS barrier maintains homeostasis in the brain and protects the central nervous system from toxins and pathogens. Dysfunction of the blood-CNS barrier is implicated in various neurological diseases. CNS endothelial cells, which form the blood-CNS barrier, are highly polarized cells held together by tight junctions that limit the flow of molecules and ions across paracellular space.

In some embodiments, blood-CNS barrier model can be produced by culturing of primary or immortalized cells from non-human sources, such as, rat, mouse, porcine, bovine, or cynomolgus monkey. Human in vitro blood-CNS barrier models are desirable, at least due to species differences in genes and proteins involved with the blood-CNS barrier and in view of the recent development of biologic drugs some of which have species selectivity against blood-CNS barrier receptors.

In some embodiments, primary CNS endothelial cells have been isolated from autopsy tissues and from patients. However, it is difficult to obtain primary CNS endothelial cells have rarely been used in in vitro human blood-CNS barrier models for drug transport evaluation due to one or more of: low cell yields, difficulties in accessing patient samples, patient-related heterogeneity, fast de-differentiation, and poor trans-endothelial electrical resistance (TEER; a measure to evaluate the integrity of the blood brain barrier).

In some embodiments, immortalized CNS endothelial cell lines can be developed using immortalization protocols involving SV-40 large-T antigen. The best characterized human BEC line is hCMEC/D3. Use of hCMEC/D3 as an in vitro BBB model was reviewed by Weksler et al., The hCMEC/D3 cell line as a model of the human blood brain barrier, Fluids Barriers CNS. 2013 Mar. 26; 10(1):16. (2013).

In some embodiments, human stem cell and/or progenitor cell sources have been used to derive CNS endothelial cells. Unlike primary CNS endothelial cell cultures, stem cells offer a theoretically unlimited expansion capacity. Human embryonic stem cells (ESCs) and human induced pluripotent stem cells (iPSCs) have been used to generate functional CNS endothelial cells expressing various blood-CNS barrier tight junction proteins, transporters, and receptors, see, e.g., Exploring the effects of cell seeding density on the differentiation of human pluripotent stem cells to brain microvascular endothelial cells, Fluids Barriers CNS. 2015; 12: 13. doi: 10.1186/s12987-015-0007-9, Lippmann et al., 2014, A retinoic acid-enhanced, multicellular human blood-brain barrier model derived from stem cell sources, Sci Rep. 2014; 4: 4160.

In some embodiments, the blood-CNS barrier phenotype of the CNS endothelial cells (e.g., stem-cell derived CNS endothelial cells) is enhanced by adding retinoic acid (RA), which induced an earlier onset of VE-cadherin expression, higher tight junction complexity, and mRNA up-regulation of multiple efflux pumps (Lippmann et al., 2012, Human Blood-Brain Barrier Endothelial Cells Derived from Pluripotent Stem Cells, Nat Biotechnol. 2012 August; 30(8): 783-791; Wilson et al., 2015, Exploring the effects of cell seeding density on the differentiation of human pluripotent stem cells to brain microvascular endothelial cells, Fluids Barriers CNS. 2015; 12: 13; Lippmann et al., 2014, A retinoic acid-enhanced, multicellular human blood-brain barrier model derived from stem cell sources, Sci Rep. 2014; 4: 4160). In some embodiments, a combination of RA treatment and co-culture of CNS endothelial cells with pericytes followed by astrocytes and neurons differentiated from neural progenitors elevated TEER levels to over 5000 Ω·cm² (Lippmann et al., 2012, Human Blood-Brain Barrier Endothelial Cells Derived from Pluripotent Stem Cells, Nat Biotechnol. 2012 August; 30(8): 783-791; Wilson et al., 2015, Exploring the effects of cell seeding density on the differentiation of human pluripotent stem cells to brain microvascular endothelial cells, Fluids Barriers CNS. 2015; 12: 13. Lippmann et al., 2014, A retinoic acid-enhanced, multicellular human blood-brain barrier model derived from stem cell sources, Sci. Rep. 2014; 4: 4160).

In other embodiments, human blood-CNS barrier model are developed from stem cell sources (e.g., iPCs). For example, human cord blood-derived hematopoietic stem cells were differentiated to endothelial cells, using Endothelium Growth Medium-2 (EGM-2; Lonza) and co-cultures of bovine pericytes to derive ‘brain-like endothelial cells (BLEC) (Cecchelli et al., 2014 PLOS ONE 9(6) e99733). Further co-culturing BLECs with pericytes from non-human species facilitated maturation of BECs towards a blood-CNS barrier phenotype, as evidenced by expression of influx transporters (SLC7A5, SLC16A1), glucose transporters (GLUT-1 or SLC2A1), several ATP-binding cassette (ABC) transporters, and other blood-CNS barrier-specific receptors.

In some aspects, the blood-CNS barrier model is produced by culturing the endothelial progenitor cells (e.g., iPSCs) in the presence of vitronectin or co-cultured with a plurality of cells secreting vitronectin. For example, endothelial progenitor cells (e.g., iPSCs) may be seeded on a Transwell insert, which may be placed in one well of a multi-well plate under conditions suitable for monolayer formation and cell growth. For example, Endothelial progenitors may be seeded at density of about between 500,000 to 1,000,000 cells/insert and may be maintained in cell culture medium during TEER measurement. Any known suitable endothelial cell culture medium can be used for the blood-CNS barrier model described herein.

### EXAMPLES

### Example I. Pericyte-to-Endothelial Cell Signaling via Vitronectin-Integrin Regulates Blood-CNS Barrier

Endothelial cells of blood vessels of the central nervous system (CNS) constitute blood-CNS barriers. Barrier properties are not intrinsic to these cells; rather they are induced and maintained by CNS microenvironment. Notably, the abluminal surface of CNS capillaries are ensheathed by pericytes and astrocytes. However, extrinsic factors from these perivascular cells that regulate barrier integrity are largely unknown. Here, vitronectin, an extracellular-matrix protein secreted by CNS pericytes, is established as a regulator of blood-CNS barrier function via interactions with its integrin receptor, α5 in endothelial cells. Genetic ablation of vitronectin or mutating vitronectin to prevent integrin binding as well as endothelial-specific deletion of integrin α5 causes barrier leakage in mice. Furthermore, vitronectin-integrin α5 signaling maintains barrier integrity by actively inhibiting transcytosis in endothelial cells.

Introduction

The CNS requires an optimal and tightly regulated microenvironment for efficient synaptic transmission. This is achieved by blood-CNS barriers that regulate substance flux to maintain tissue homeostasis. Two such barriers are the blood-brain barrier (BBB) and blood-retina barrier (BRB) that are physiologically similar barriers separating the blood from brain and retina, respectively. The restrictive permeability of CNS endothelial cells that constitute these barriers is a result of tight junctions and low rates of transcytosis, which limit substance exchange between blood and the CNS tissue (Andreone et al., 2017; Ben-Zvi et al., 2014; Chow and Gu, 2017; Langen et al., 2019; Reese and Karnovsky, 1967; Zhao et al., 2015).

Barrier properties are not intrinsic to CNS endothelial cells; they require active induction and maintenance from brain parenchyma cells (Armulik et al., 2010; Daneman et al., 2010; Heithoff et al., 2021; Stewart and Wiley, 1981). For example, Wnt ligands released by glia and neurons act on CNS endothelial cells to induce and maintain barrier properties (Daneman et al., 2009; Liebner et al., 2008; Stenman et al., 2008; Wang et al., 2012, 2019b). In contrast to glia and neurons, pericytes are directly in contact with capillary endothelial cells. Pericytes ensheathe capillary endothelial cells and share the same basement membrane with them allowing for elaborate cell-cell signaling between these two cells. Intriguingly, the brain and retina have the highest pericyte to endothelial cell ratio compared to that in other tissues (Frank et al., 1987; Shepro and Morel, 1993). Indeed, mice with decreased pericyte coverage around endothelial cells exhibit leaky blood-CNS barriers (Armulik et al., 2010; Bell et al., 2010; Daneman et al., 2010; Park et al., 2017). However, how pericytes signal to endothelial cells to maintain barrier integrity is unknown. Vitronectin, a pericyte-secreted extracellular matrix (ECM) protein is identified as an important regulator of barrier integrity. The present disclosure establishes that vitronectin regulates barrier function via binding to its integrin receptors on endothelial cells. Vitronectin is enriched in CNS pericytes, and mice lacking vitronectin as well as vitronectin mutant mice (Vtn^{RGE}) that cannot bind integrin receptors exhibit barrier leakage. Moreover, the RGD-ligand binding integrins, α5 and αv are expressed in CNS endothelial cells, and endothelial cell-specific acute deletion of α5 but not αv results in leaky barrier. The results further demonstrate that barrier leakage observed in vitronectin mutant mice is due to increased transcytosis in CNS endothelial cells, but not due to deficits in tight junctions; and activation of integrin α5 by vitronectin inhibits transcytosis in CNS endothelial cells.

Vitronectin is Enriched in CNS Pericytes Compared to Pericytes of Peripheral Tissues and Its Expression Coincides with Functional Barrier Formation

The retina was used as a model system in order to identify pericyte candidate genes important for barrier function. In the retinas of mice, CNS vessels invade the optic nerve head at postnatal day (P1) and expand radially from the center toward the periphery. As vessels grow from the proximal to distal ends of the retina, proximal vessels gain barrier properties and have a functional BRB, while the newly formed distal vessels have a leaky BRB (Chow and Gu, 2017). By examination of an existing transcriptome comparing the proximal and distal retinal vessels that contain a mixture of endothelial cells and pericytes (Strasser et al., 2010) and a brain pericyte transcriptomic database (He et al., 2016), CNS pericyte genes that correlated with functional blood-retinal barrier were identified. Of these candidate pericyte genes, secreted and trans-membrane proteins were of interest as these were likely involved in ligand-receptor interactions. These analyses identified vitronectin, an extracellular matrix protein.

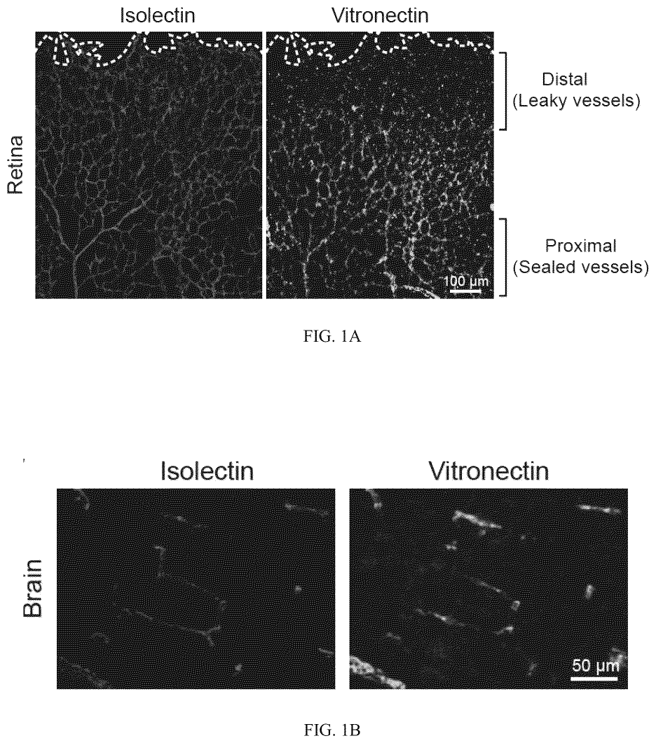

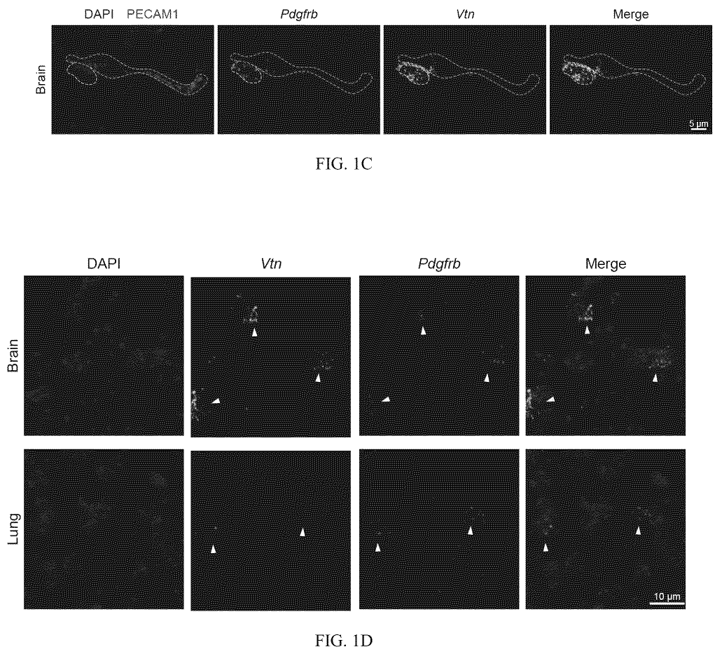

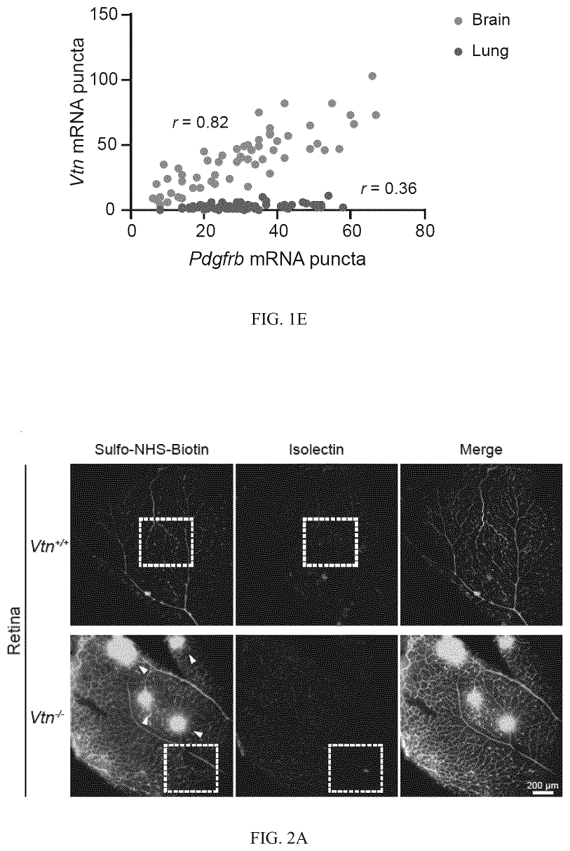

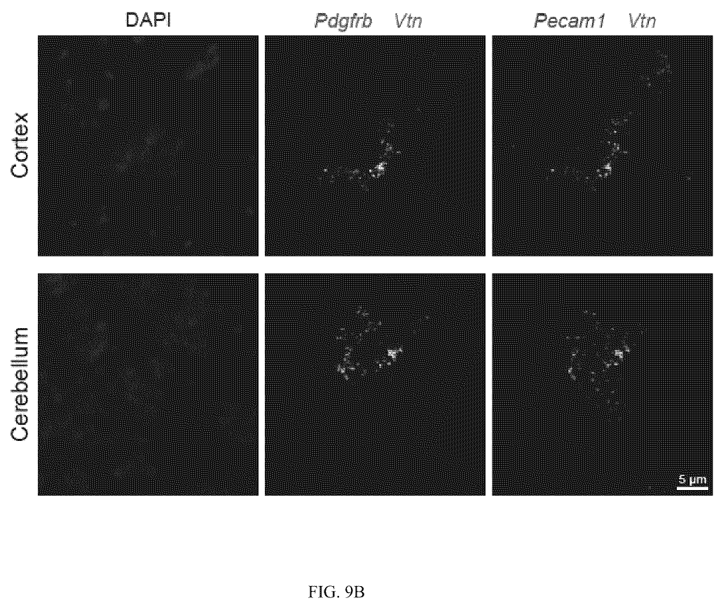

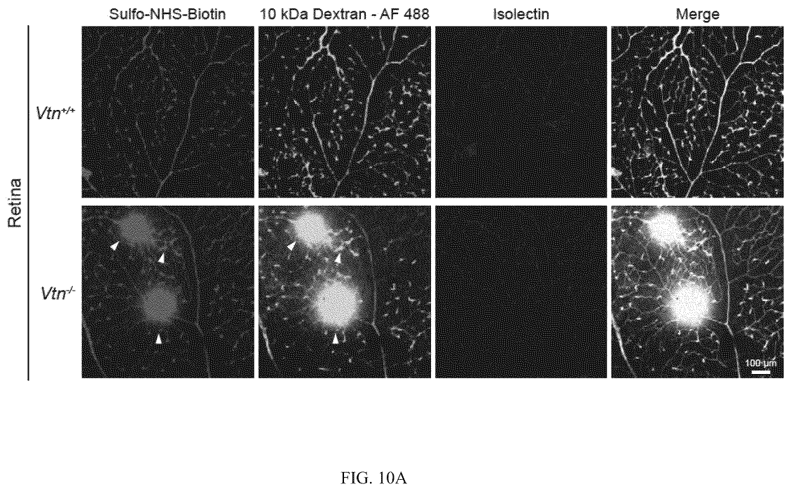

To validate the gene expression analyses, vitronectin protein localization was first examined in the CNS. Consistent with enriched Vtn transcript in proximal compared to distal vessels of the developing retina (Strasser et al., 2010), it was observed that vitronectin protein was highly expressed in proximal vessels with sealed BRB, and was not detectable in the distal, leaky vessels of the retina at P7 (FIGS. 1A, 9A). Similarly, vitronectin protein was also detected along capillaries of the brain (FIG. 1B). As vitronectin is a secreted protein, in situ hybridization was used to examine Vtn mRNA in the brain to determine which cells produced Vtn. Vtn mRNA was specifically expressed in Pdgfrb expressing pericytes adjacent to capillary endothelial cells (FIG. 1C). At P7, Vtn was expressed in >97% (115 out of 118 cells across 3 mice) of the Pdgfrb+ pericytes abutting capillaries and was present throughout the brain (FIGS. 1C, 9B). In contrast to high Vtn mRNA in CNS pericytes, little to no expression in pericytes of peripheral tissues, such as the lung (FIG. 1D and FIG. 1E) was observed. The data was consistent with recent single-cell RNA sequencing studies revealing abundant Vtn expression in P14 retinal pericytes (Macosko et al., 2015; La Manno et al., 2021; Vanlandewijck et al., 2018). This was also consistent with single cell-RNA sequencing studies in adult brains with Vtn expression in pericytes but little to no expression in non-vascular CNS cells, such as neurons and astrocytes (La Manno et al., 2021; Vanlandewijck et al., 2018).

Pericyte-Secreted Vitronectin is Required for Blood-CNS Barrier Integrity

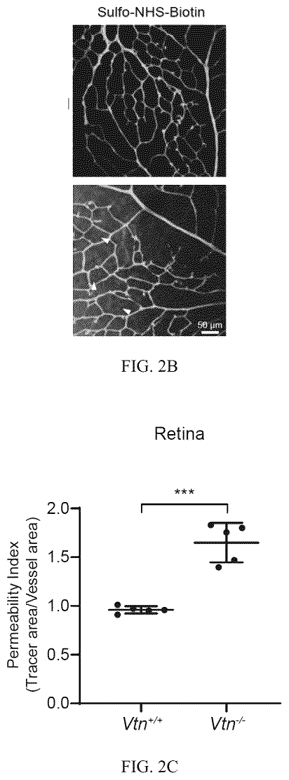

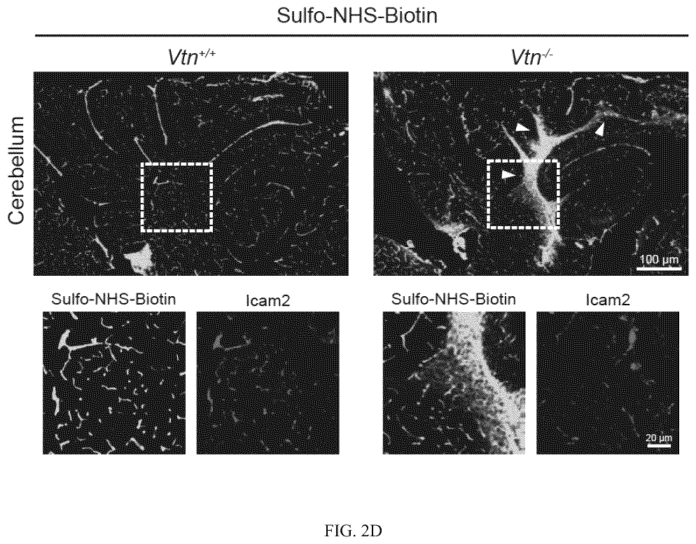

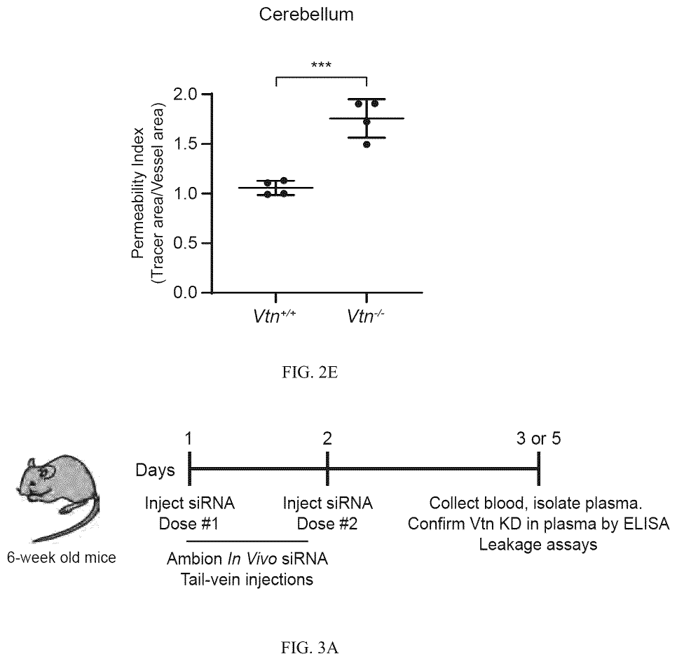

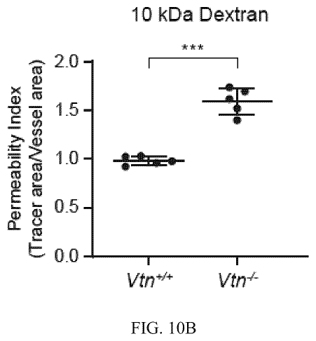

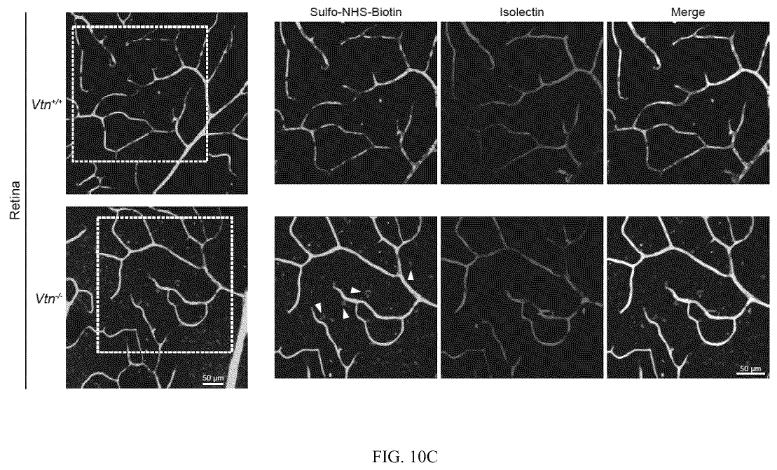

To determine if vitronectin was essential for barrier function, a tracer leakage assay was performed in P10 Vtn^{-/-} mice when BRB and BBB were fully functional (Andreone et al., 2017; Chow and Gu, 2017). The injected tracer was completely confined to vessels in control mice, whereas tracer leaked out of vessels in the retinas of Vtn^{-/-} mice. Numerous leaky hotspots of the tracer were apparent in the parenchyma (FIG. 2A) and in neuronal cell bodies (FIG. 2B) in the retinas of these mice (FIG. 2C). This leakage was not limited to just small tracers like Sulfo-NHS-Biotin (0.5 kDa) as we observed similar leakage with higher molecular weight tracers such as 10 kDa dextran (FIGS. 10A, 10B). Furthermore, this leakage persisted through adulthood in these mice (FIG. 10C). Similar to the retina, BBB leakage in the cerebellum was observed (FIGS. 2D, 2E).

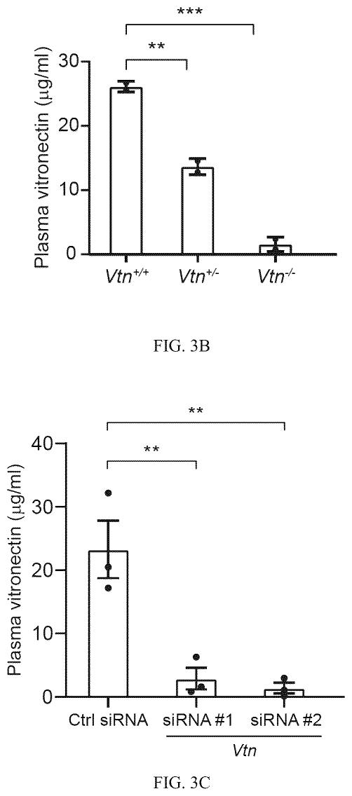

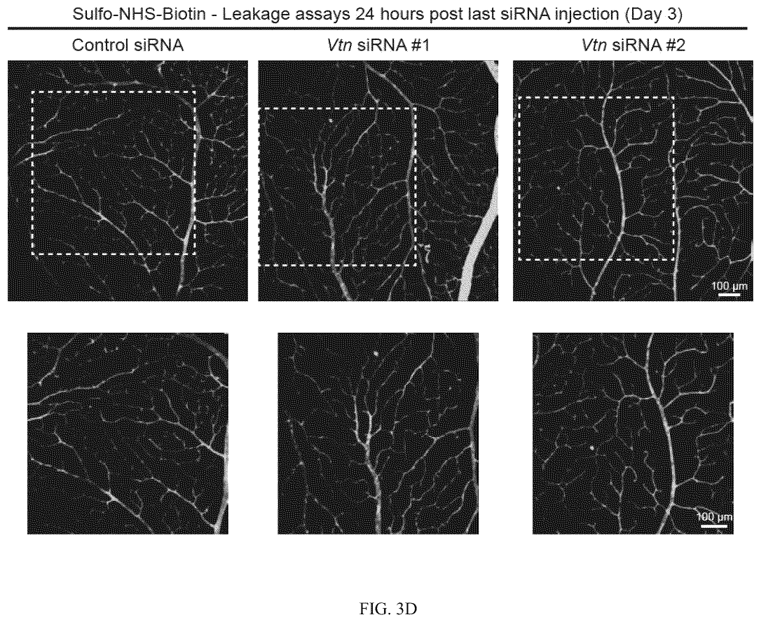

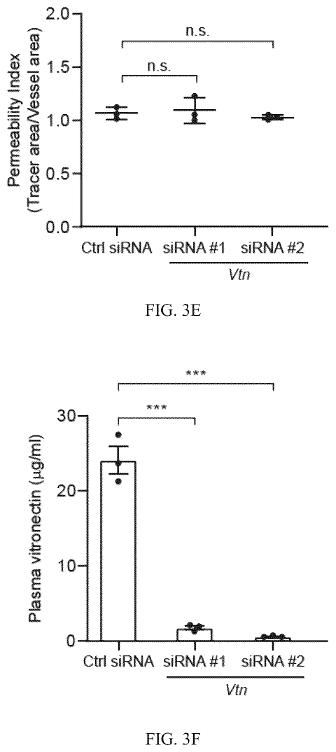

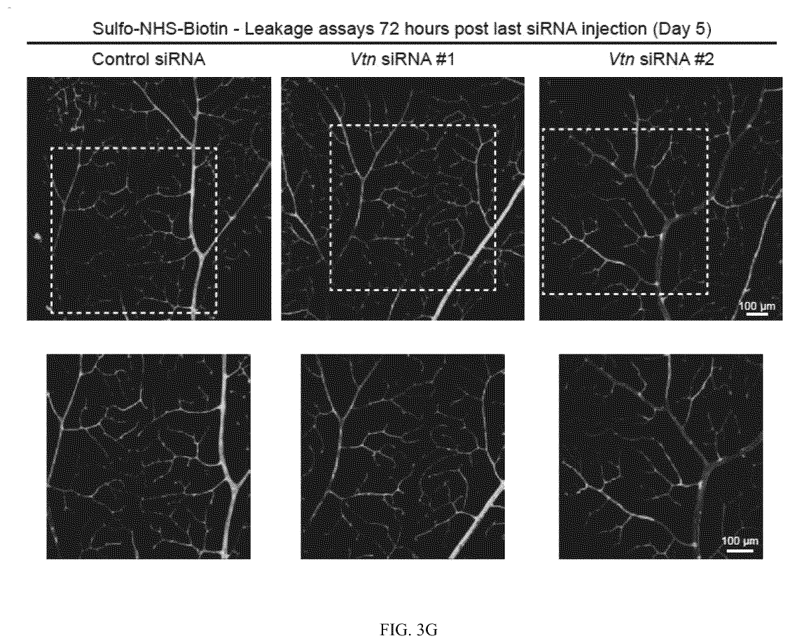

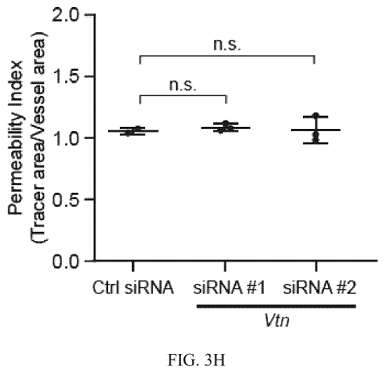

In addition to being an ECM protein, vitronectin is also an abundant protein circulating in the plasma which has been shown to mediate the complement pathway and play a role in tissue repair and wound healing (Leavesley et al., 2013; Preissner and Reuning, 2011). Utilizing the fact that vitronectin in plasma is synthesized and secreted by liver hepatocytes and that intravenous injections of siRNAs largely target the liver tissue with little to no distribution to other tissues (Song et al., 2003; Zender et al., 2003), it was established that the barrier deficits observed in Vtn^{-/-} mice were due to the lack of pericyte-secreted vitronectin and not due to the lack of circulating vitronectin. To specifically knockdown the circulating vitronectin, siRNAs targeting vitronectin or control siRNAs were intravenously injected into 6-week-old wildtype mice on two consecutive days (FIG. 3A) based on previously established studies for efficient liver targeting (Song et al., 2003; Wrobel et al., 2021). Knockdown of vitronectin in plasma was evaluated by ELISA at day 3 or 5, i.e., 24 hours or 72 hours post the last dose of siRNA injections (FIG. 3A). ELISA kit was validated by measuring levels of plasma vitronectin in wild-type, Vtn^{+/-}, and Vtn^{-/-} mice (FIG. 3B). These experiments were performed with two independent siRNAs at both the timepoints. A knockdown as high as 98.56±0.06% of vitronectin in plasma isolated from mice injected with vitronectin targeting siRNAs (FIGS. 3C, 3F) was observed. Importantly, at both these timepoints, no leakage was detected in mice injected with either siRNA targeting vitronectin or control siRNA (FIGS. 3D, 3E, 3G, 3H), demonstrating that plasma vitronectin is likely dispensable for barrier function.

Vitronectin Regulates Blood-CNS Barrier Function by Suppressing Transcytosis in CNS Endothelial Cells

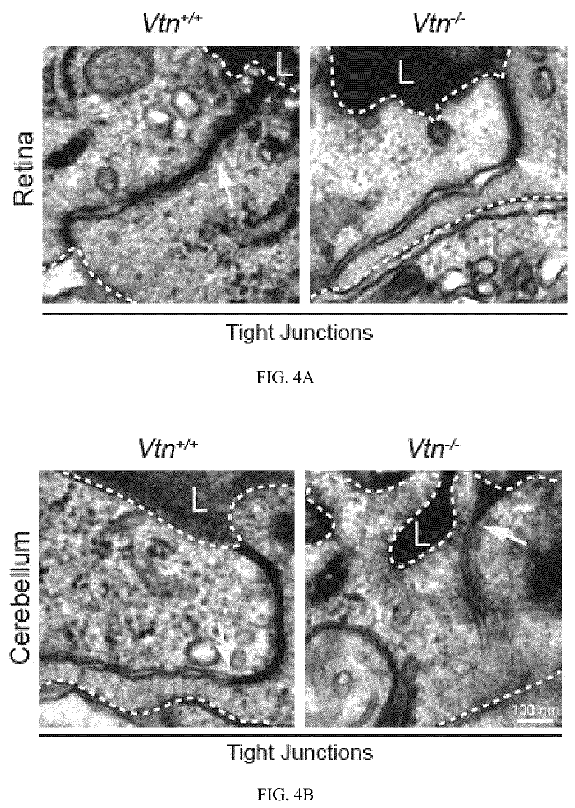

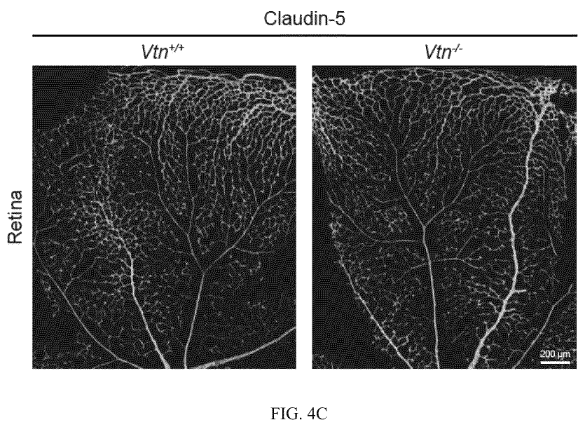

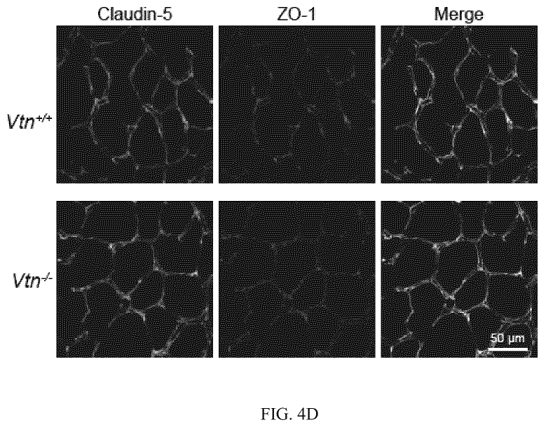

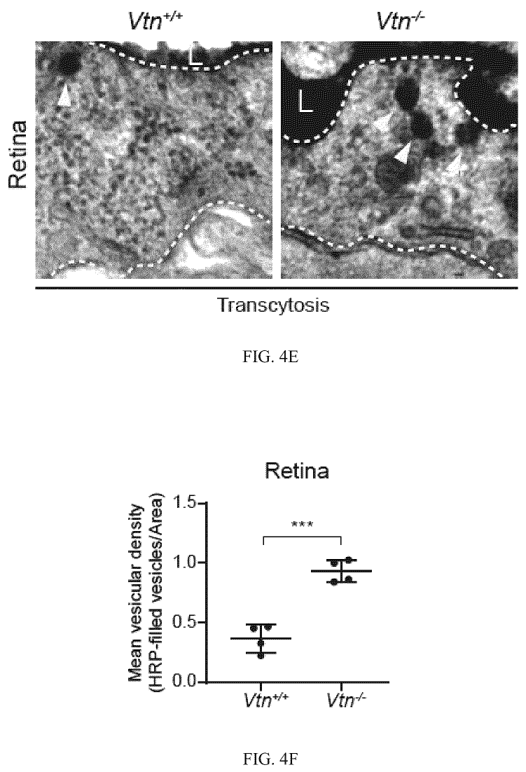

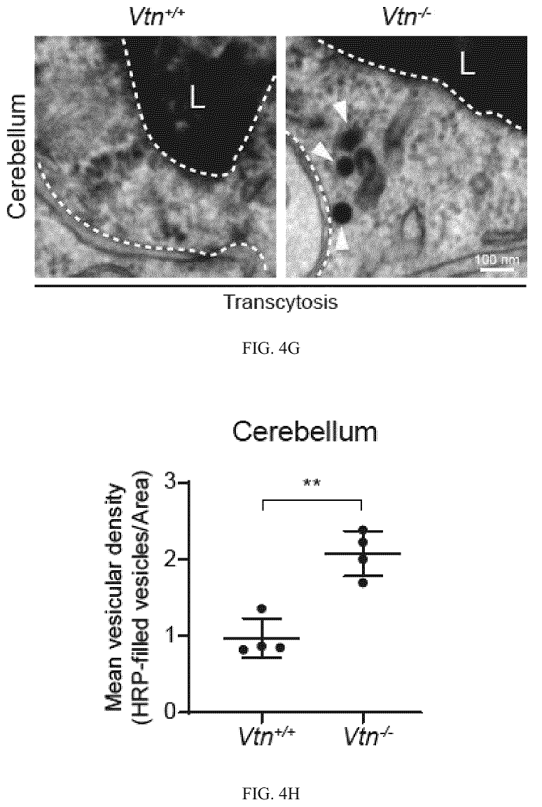

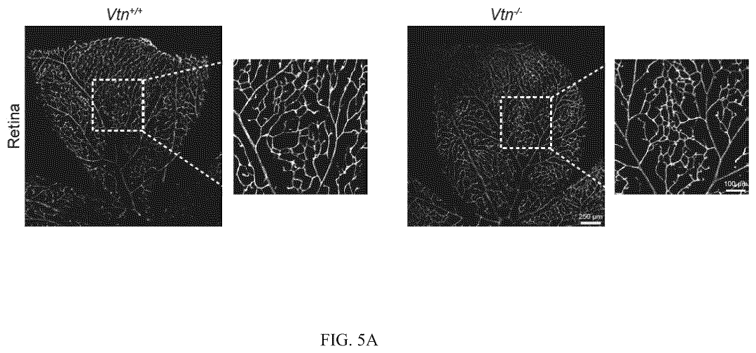

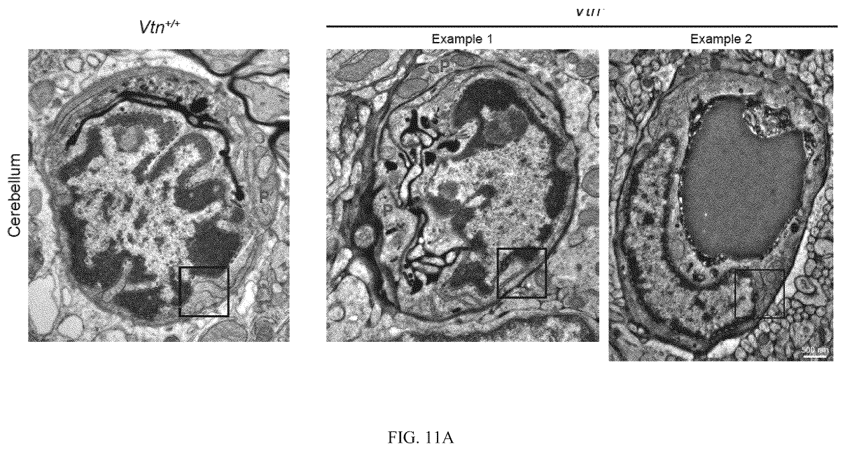

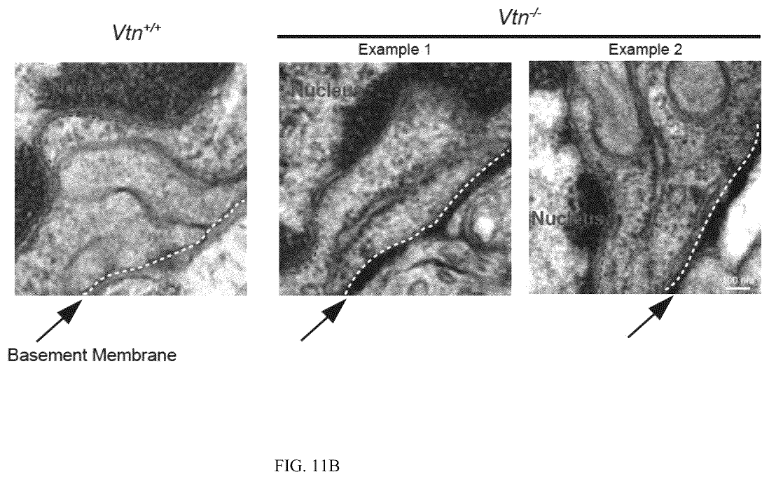

To determine the subcellular basis in endothelial cells for the underlying leakage observed in Vtn^{-/-} mice, horse-radish peroxidase (HRP) was injected intravenously and EM analysis was performed in the retina and cerebellum. In both retina and cerebellum of Vtn^{-/-} mice and wildtype littermates, HRP was observed halting at tight junction “kissing points” between endothelial cells of capillaries (FIGS. 4A-4B), indicating functional tight junctions. Consistent with this, no changes were observed in Claudin-5 protein expression in the retinas of Vtn^{-/-} mice (FIG. 4C). Closer examination of Claudin-5 and ZO-1 revealed that the localization of these proteins to cell-cell junctions was also unaltered (FIG. 4D). In contrast, both retinal and cerebellum endothelial cells of Vtn^{-/-} mice exhibited significantly increased HRP-filled vesicles (FIGS. 4E, 4G). Retinal endothelial cells had a 3-fold increase in vesicles compared to wildtype mice (FIG. 4F) and cerebellar endothelial cells had 2-fold increase (FIG. 4H), revealing that transcytosis was upregulated in these mice. Interestingly, in the cerebellum of these mice, it was also observed that HRP-filled vesicles in adjacent pericytes (FIG. 11A) and about 25% of capillaries had their basement membrane completely filled with HRP (FIGS. 11B, 11C). This result indicates that tracer-filled vesicles were indeed transcytosed across endothelial cells (FIG. 11A). These data reveal that barrier leakage in Vtn^{-/-} mice was due to upregulated transcytosis. Together, these observations demonstrate that pericyte-derived vitronectin regulates barrier function at least by inhibiting transcytosis in CNS endothelial cells.

Vitronectin is Not Required for Normal Vessel Patterning or Pericyte Coverage

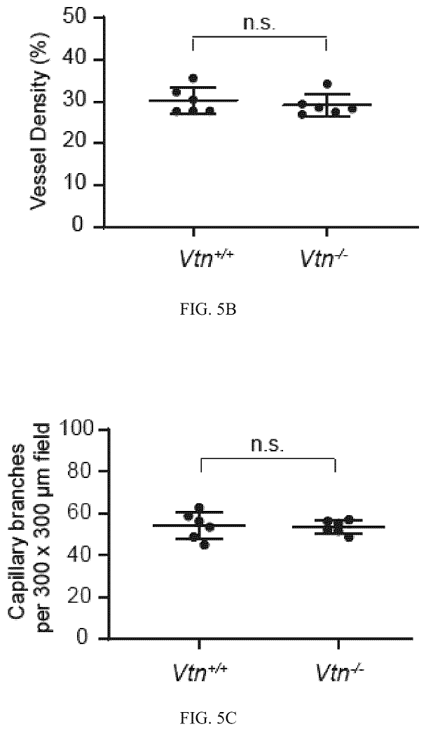

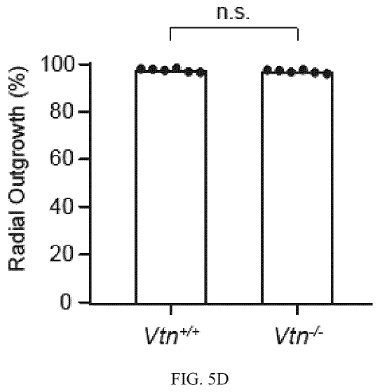

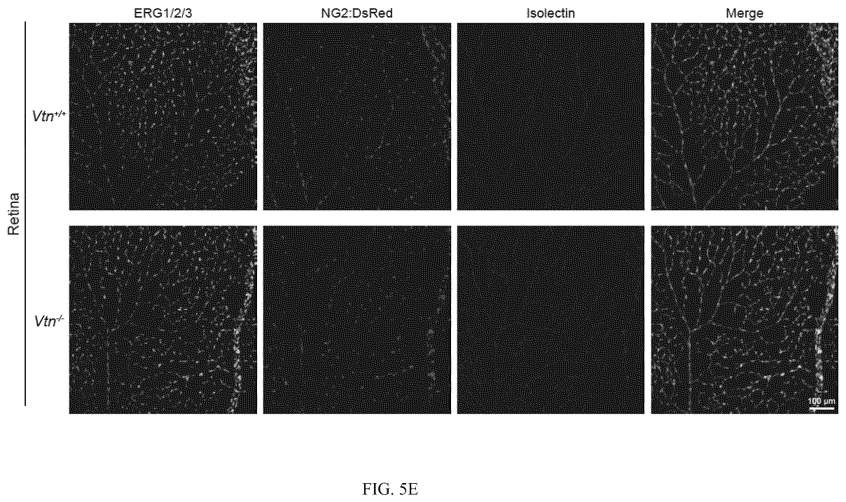

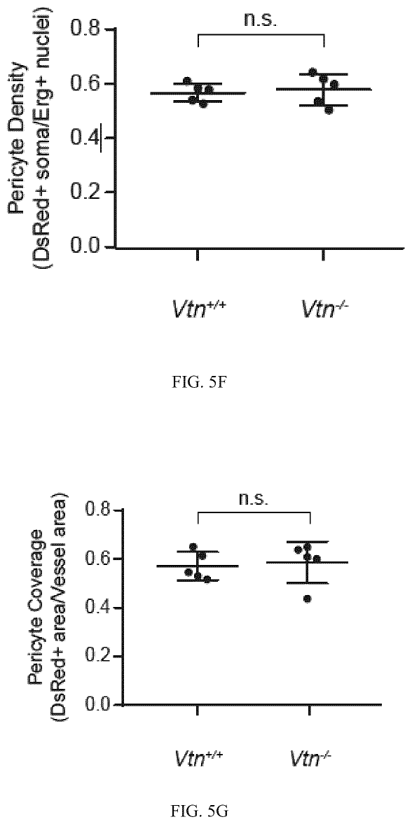

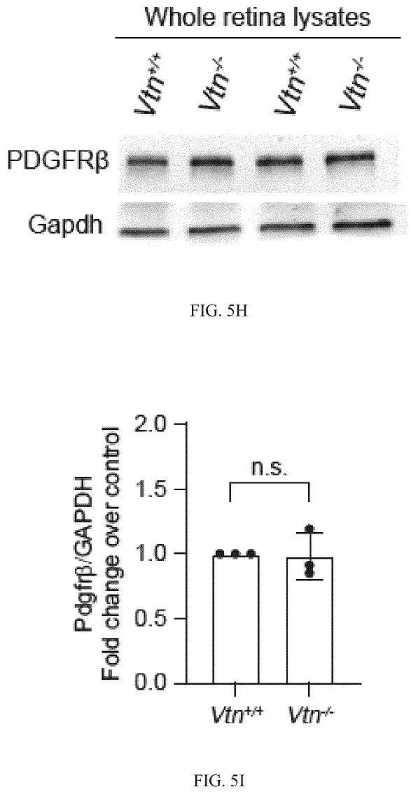

Next, it was considered how vitronectin, a pericyte secreted protein, regulated barrier properties in endothelial cells. One possibility was that barrier defects were due to impaired vessel development as pericytes are known to regulate endothelial sprouting and angiogenesis in the postnatal retinal vasculature (Eilken et al., 2017). However, no changes in vascular patterning were observed (FIG. 5A). Specifically, no changes in vessel density, capillary branching or radial outgrowth of the vasculature in Vtn^{-/-} mice compared to wildtype littermates was observed (FIGS. 5B-5D).

Another possibility was that, since vitronectin is a pericyte gene and pericyte-deficient mice exhibit leaky barriers (Armulik et al., 2010; Daneman et al., 2010), barrier defects could be due to altered pericyte coverage. However, using NG2:DsRed mice that label mural cells (FIG. 5E), similar pericyte coverage and pericyte density were observed in Vtn^{-/-} and wildtype mice (FIGS. 5F, 5G). PDGFRβ protein levels were also similar in Vtn^{-/-} and wildtype retinas (FIGS. 5H, 5I). Hence, vitronectin regulated barrier function without altering pericyte density or pericyte coverage.

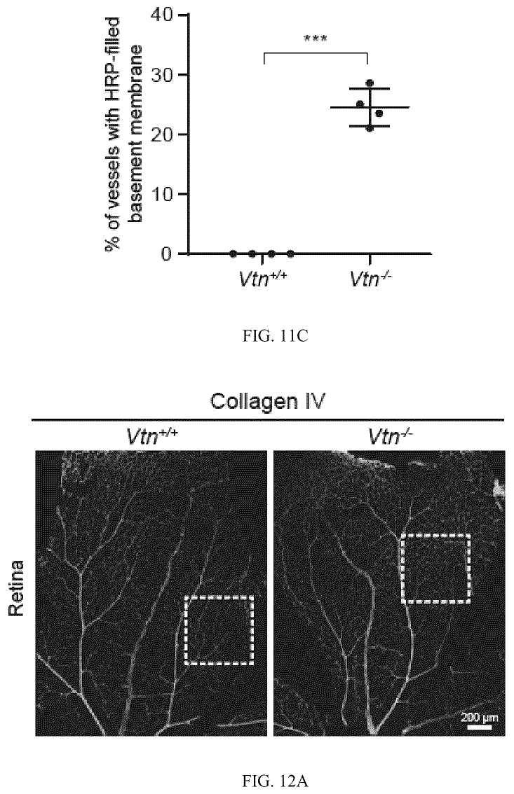

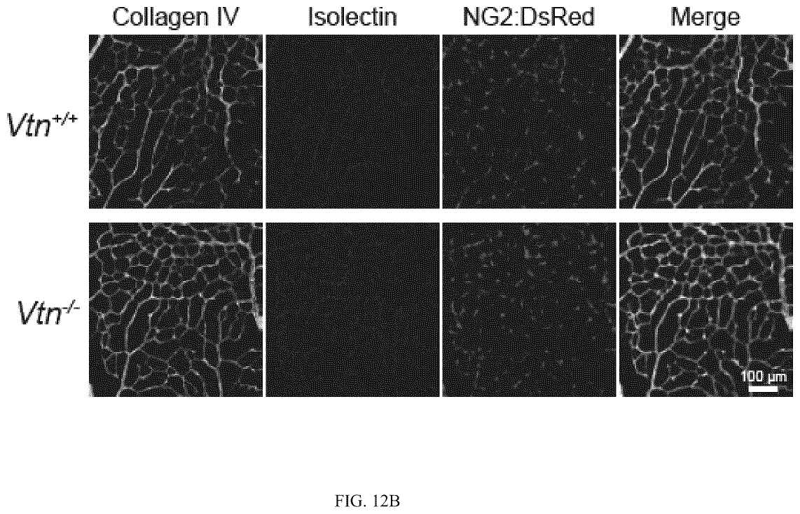

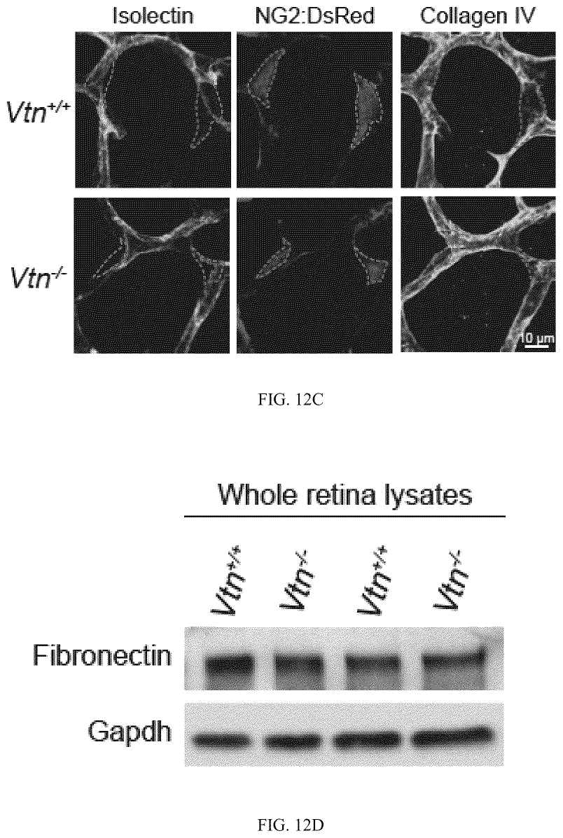

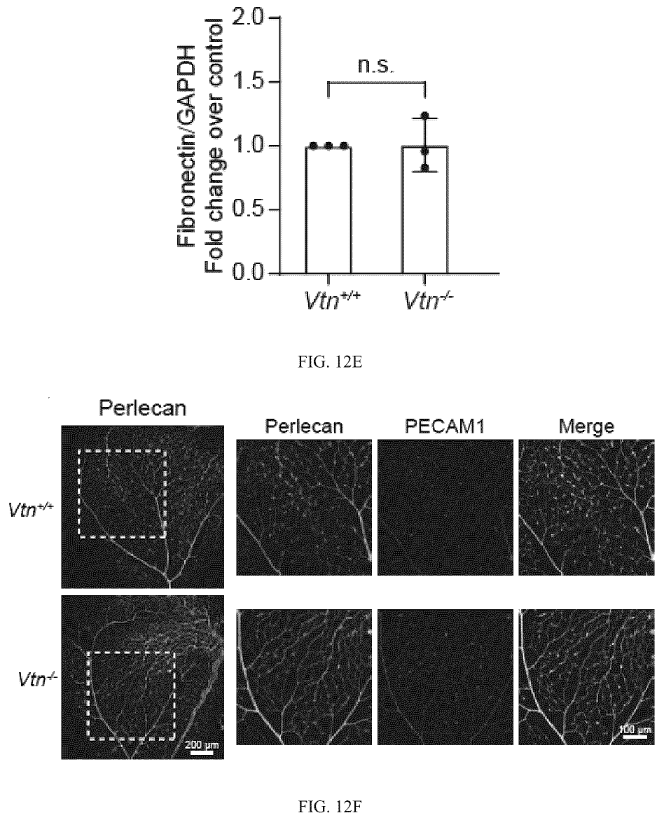

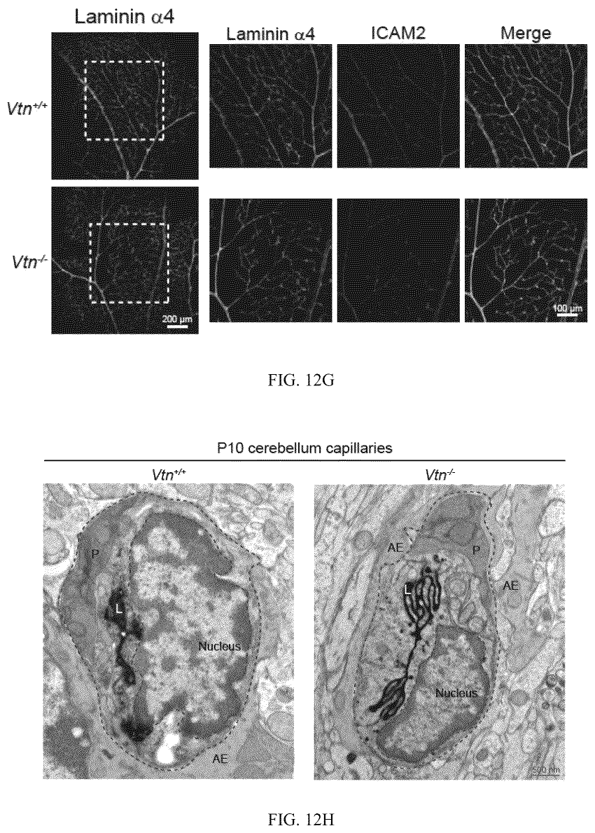

Since vitronectin is an ECM protein, it was assessed whether the barrier deficits in Vtn^{-/-} mice were a consequence of structural and/or functional changes in the ECM. EM analysis of retinal and cerebellum capillaries revealed no obvious structural changes in the basement membrane of endothelial cells in Vtn^{-/-} mice compared to wildtype mice. Moreover, immunostaining and western blotting of two highly enriched ECM proteins, collagen IV and fibronectin, revealed normal localization (FIGS. 12A, 12B) and expression levels (FIGS. 12D, 12E) of these proteins in retinas of Vtn^{-/-} mice. Notably, normal collagen IV ensheathment of retinal blood vessels in Vtn^{-/-} mice was observed (FIG. 12C). Similarly, there was normal localization and expression of two other ECM proteins, perlecan and laminin α4, in the retina vasculature of Vtn^{-/-} mice (FIGS. 12F, 12G). EM data was used to investigate astrocyte endfeet attachments and no changes were observed in the average area of cerebellum capillaries covered by astrocyte endfeet between wildtype and Vtn^{-/-} mice (FIGS. 12H, 12I). Thus, the overall structural and functional organization of the vascular basement membrane or the ECM was not compromised in mice lacking vitronectin.

Collectively, the data demonstrated that barrier defects caused in mice lacking vitronectin were not due to defects in vessel morphology and patterning, pericyte coverage, ECM organization, or astrocyte endfeet. Rather, pericyte-secreted vitronectin could be acting directly on endothelial cells to regulate their barrier properties.

### Vitronectin Binding to Integrin Receptors Induces and Maintains Barrier Function

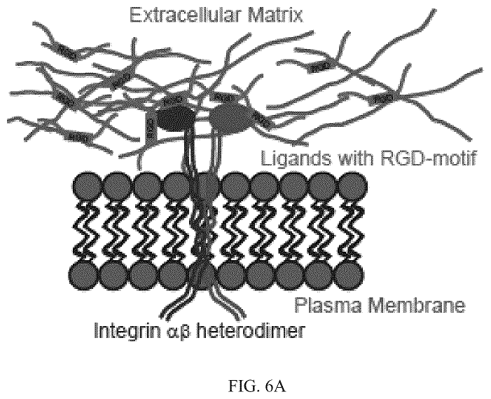

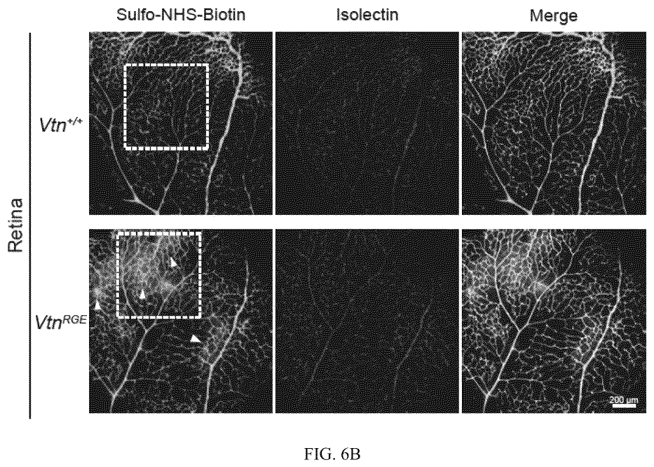

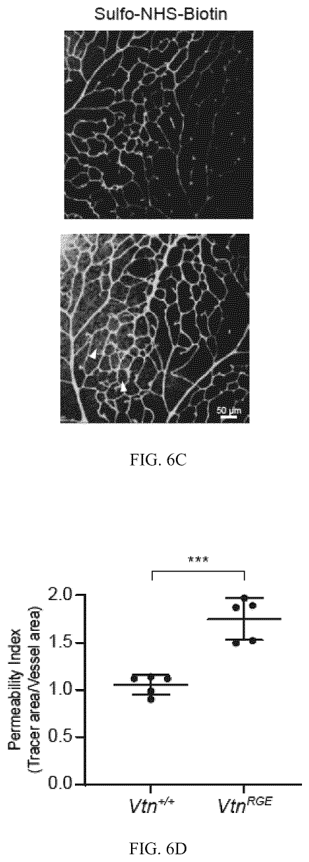

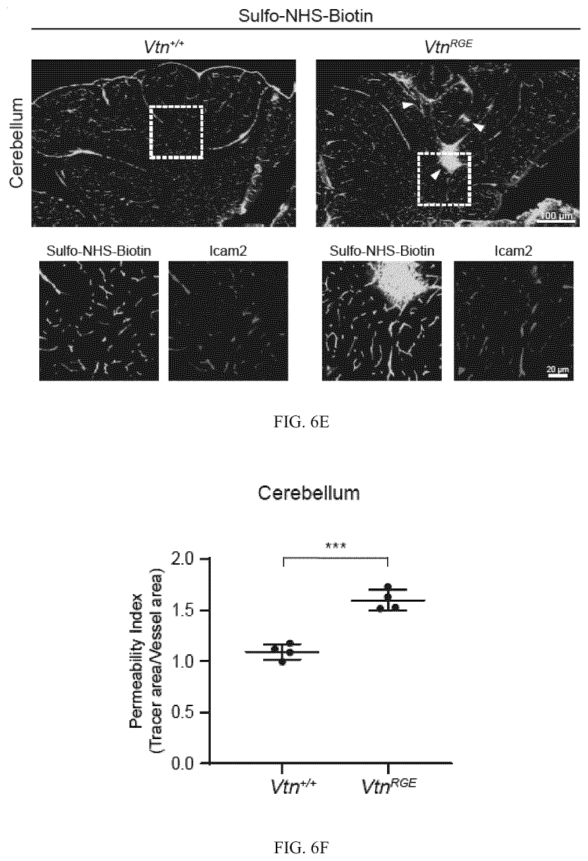

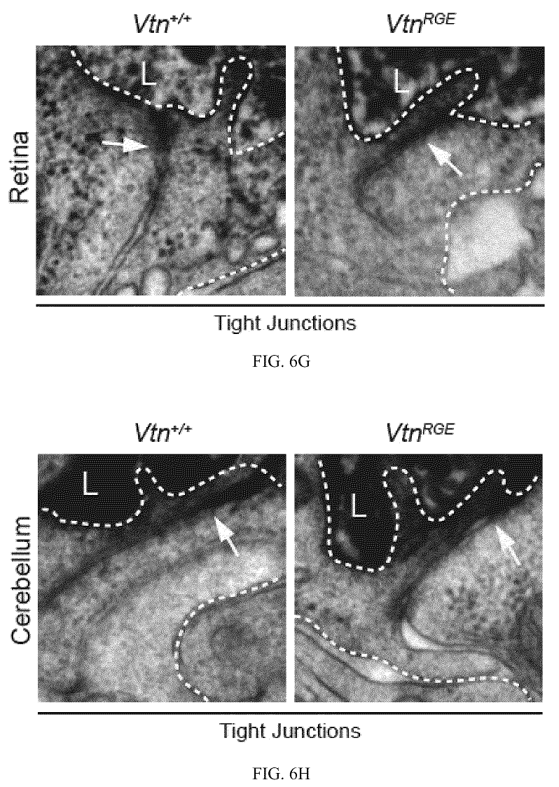

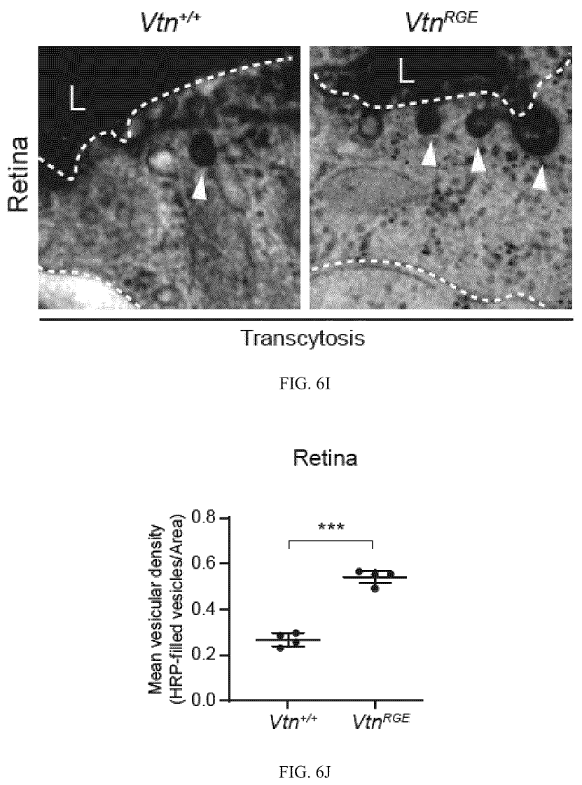

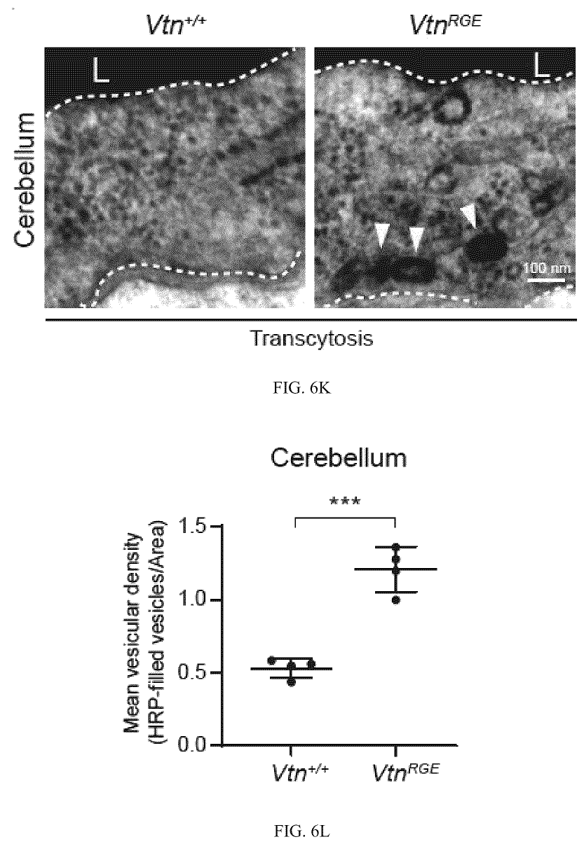

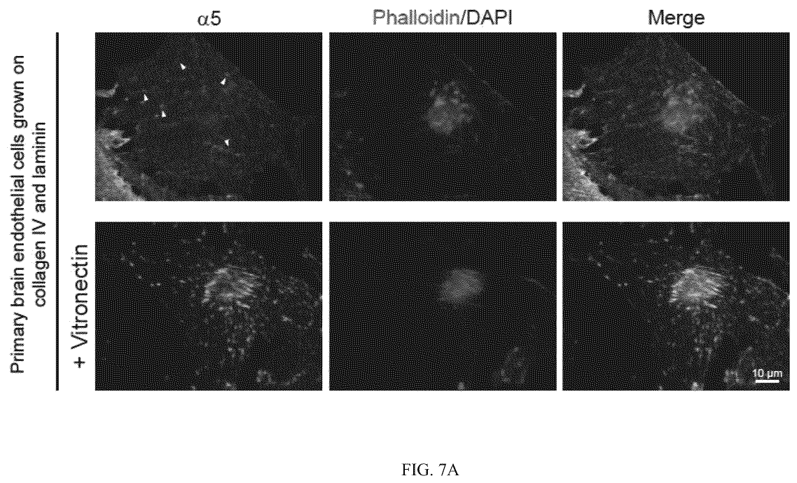

It was then determined how pericyte secreted vitronectin signaled to neighboring endothelial cells in order to regulate barrier permeability. Vitronectin belongs to the family of adhesion proteins that bind integrin receptors through a 3 amino acid motif, Arg-Gly-Asp (RGD) (Hynes, 2002; Preissner, 1991; Preissner and Reuning, 2011) (FIG. 6A). Previous biochemical studies established that competitive binding experiments using short peptides containing the RGD domain (Orlando and Cheresh, 1991) as well as mutating vitronectin RGD to RGE (aspartic acid to glutamic acid) effectively abolished binding of vitronectin to integrins (Cherny et al., 1993). To determine if vitronectin binding to integrins included and maintained for barrier function, the knock-in mouse line harboring single point mutation of vitronectin with RGD domain mutated to RGE, Vtn^{RGE/RGE} (Wheaton et al., 2016), hereafter referred to as Vtn^{RGE}, was used.

Vtn^{RGE} mice exhibited leakage in retina and cerebellum, phenocopying Vtn^{-/-} mice. Similar hotspots of tracer leakage (FIG. 6B) as well as neuronal cell bodies filled with tracer were apparent in the retinal parenchyma of Vtn^{RGE} mice (FIGS. 6C, 6D) with no obvious defects in vascular patterning. The BBB in the cerebellum of these mice was also leaky with tracer hotspots throughout the cerebellum (FIGS. 6E, 6F).

EM analysis of retinas and cerebellum of HRP-injected Vtn^{RGE} mice similar subcellular features in endothelial cells as observed in the Vtn^{-/-} full knockout mice. Both the retina and cerebellum in Vtn^{RGE} mice had functional tight junctions as noted by HRP halting sharply between endothelial cells (FIGS. 6G, 6H). However, there was a >2-fold increase in HRP-filled vesicles in endothelial cells of Vtn^{RGE} mice compared to wildtype animals (FIGS. 6I, 6J, 6K, 6L), revealing upregulated transcytosis in these mice. Thus, Vtn^{RGE} mice fully recapitulated the phenotype observed in Vtn^{-/-} mice, indicating vitronectin binding to integrin receptors regulates barrier function.

Vitronectin-Integrin α5 Signaling Inhibits Endocytosis in CNS Endothelial Cells

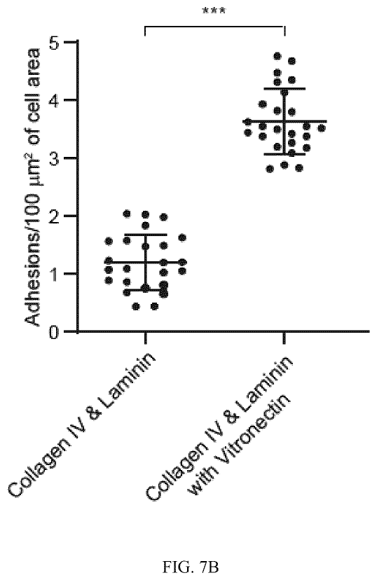

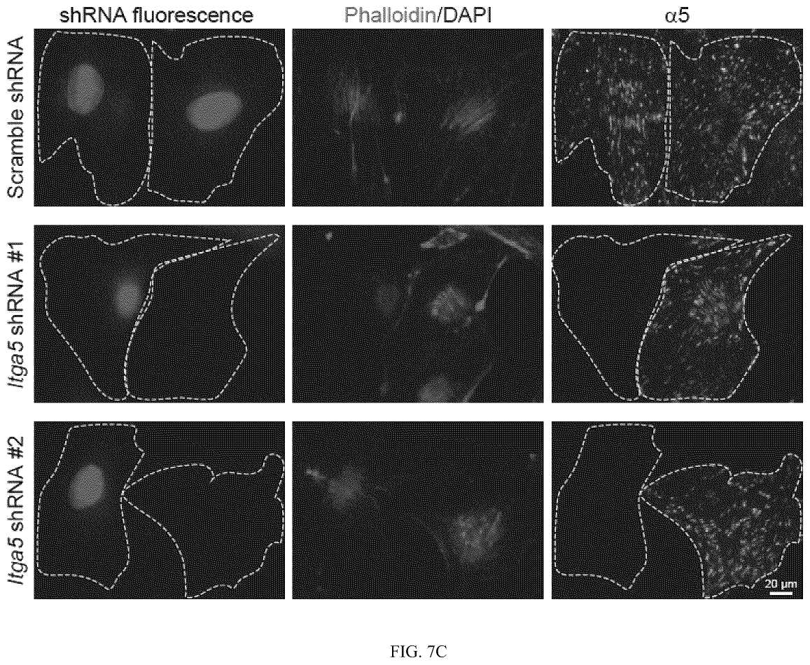

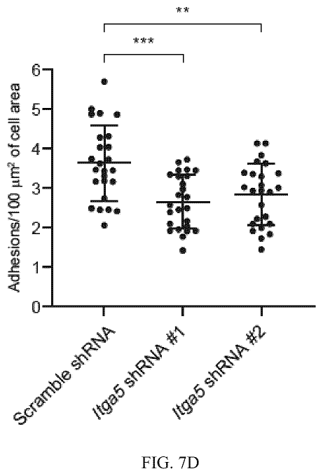

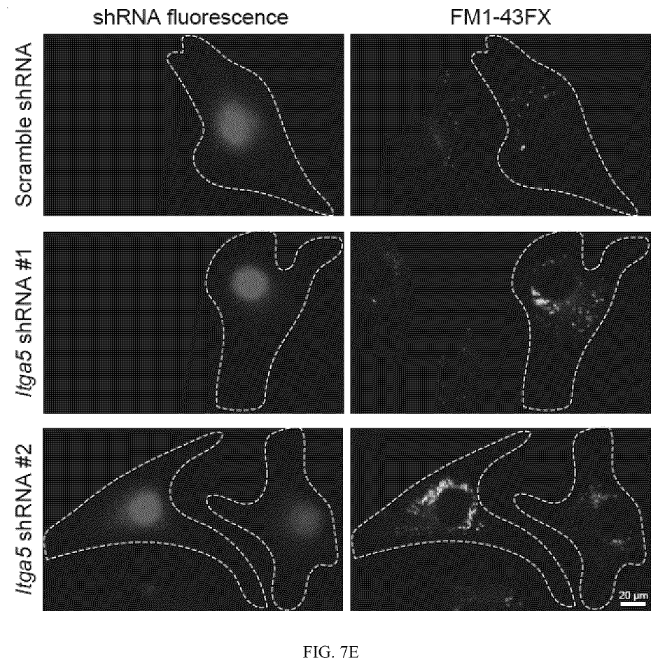

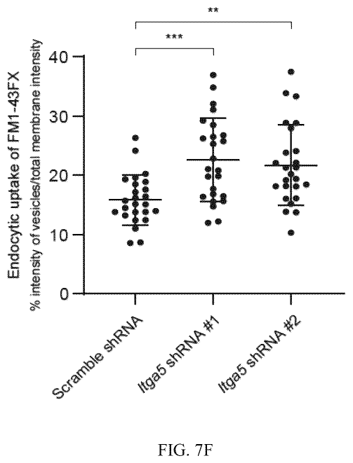

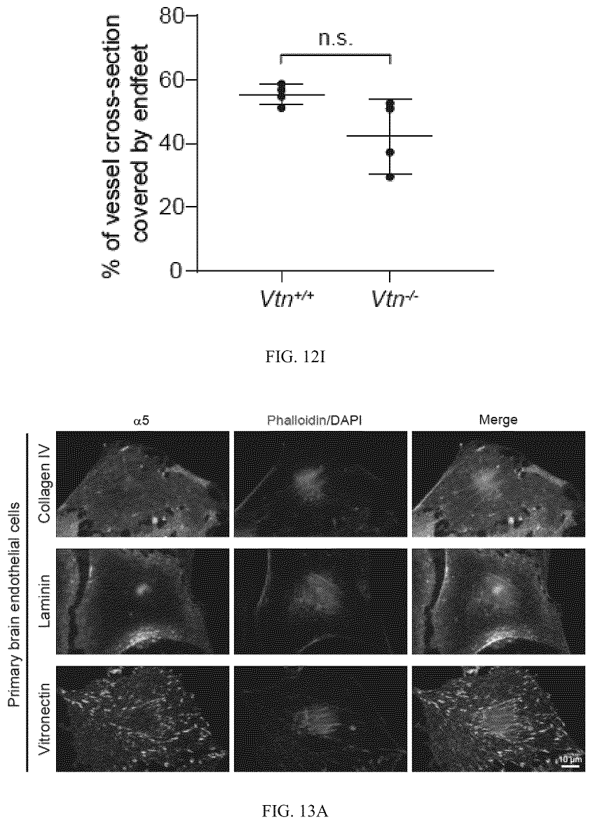

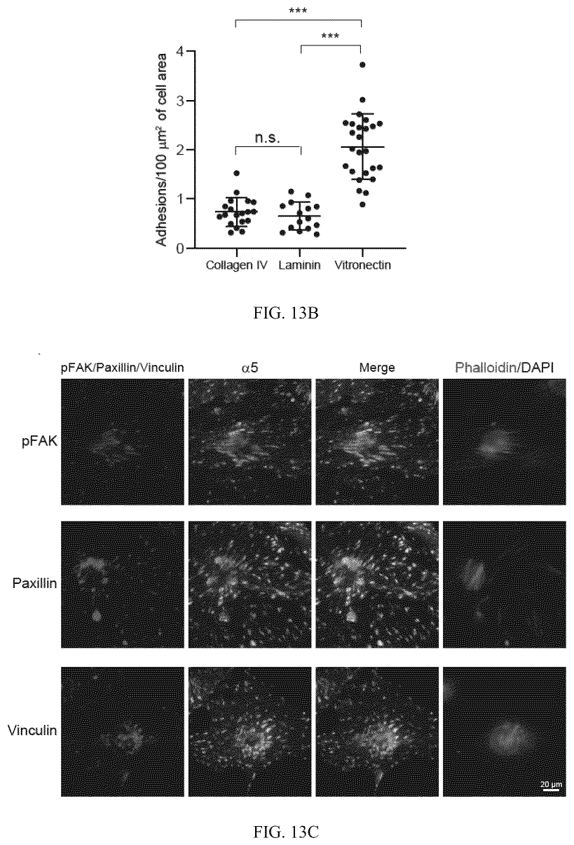

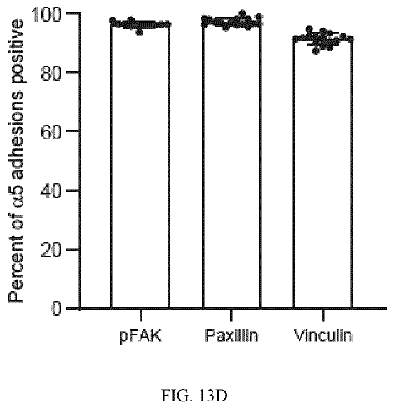

Various integrin receptors on endothelial cells were assessed to determine which specific receptors were required for vitronectin-mediated regulation of barrier function. Of the several α and β integrin subunits that heterodimerize to form integrin receptors (Hynes, 2002) two α receptors, α5 and αv, were focused on as these have been shown to interact with RGD-ligands. While αv is considered the predominant receptor for vitronectin, cell motility studies have also demonstrated interaction of vitronectin with α5 (Bauer et al., 1992; Zhang et al., 1993). Indeed, when primary mouse brain endothelial cells were grown on vitronectin-coated dishes, adhesive structures containing endogenous integrin α5 (FIG. 13A) were observed. In contrast, no adhesion structures were observed in cells grown on collagen IV coated or laminin-511 (α5β1γ1, hereafter referred to as laminin) coated dishes, demonstrating specific binding and activation of integrin α5 by vitronectin (FIG. 13A). To mimic and recapitulate the in vivo ECM as much as possible, these cells were also grown on a combination of ECM ligands. Similar to the findings with individual ligands, integrin α5 containing adhesion structures only in the presence of vitronectin (FIGS. 7A, 7B) were observed. Furthermore, >95% of α5 adhesions were positive for and co-localized with classic focal adhesion proteins such as phosphorylated FAK (Y397), paxillin, and vinculin (FIGS. 13C, 13D), indicating that α5 adhesions are indeed bonafide focal adhesion structures. Thus, vitronectin actively engages integrin α5 receptors in CNS endothelial cells and drives the formation of α5 containing focal adhesions.

Since deletion of vitronectin results in barrier leakage due to increased transcytosis, the role of vitronectin-integrin α5 interactions in vesicular trafficking in primary brain endothelial cells was subsequently investigated. Knockdown of integrin α5 in these cells with two independent shRNAs (FIGS. 7C, 7D), resulted in a significant increase in endocytosis (FIGS. 7E, 7F). In cells depleted of integrin α5 by either shRNA, FM1-43FX dye was readily incorporated in the newly formed vesicles that were endocytosed from plasma membrane, whereas very few FM1-43FX dye-positive vesicles were observed within the cells transfected with scrambled shRNA. These results demonstrated that the engagement of abluminal vitronectin-integrin α5 actively inhibits transcytosis in CNS endothelial cells.

Integrin Receptor, α5 in Endothelial Cells Regulates Blood-CNS Barrier Integrity

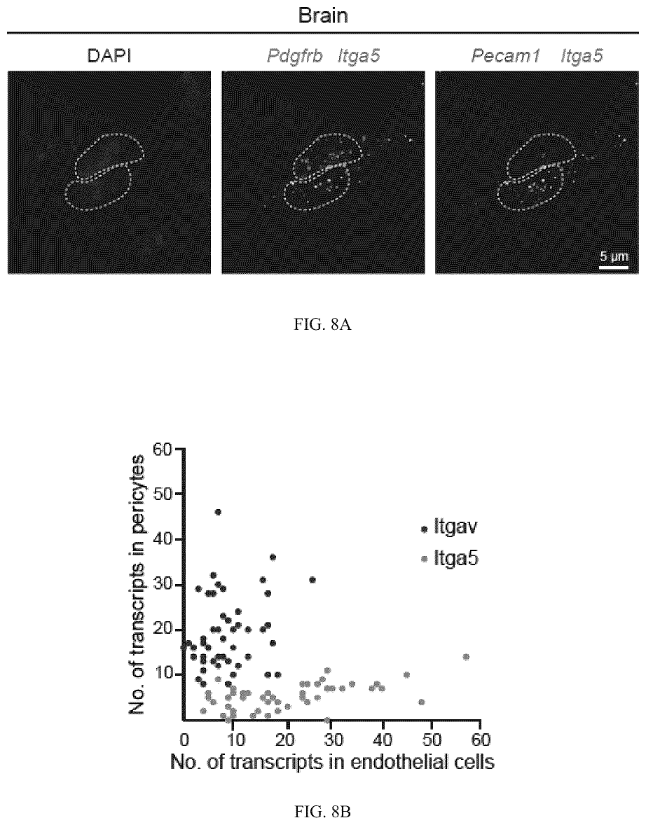

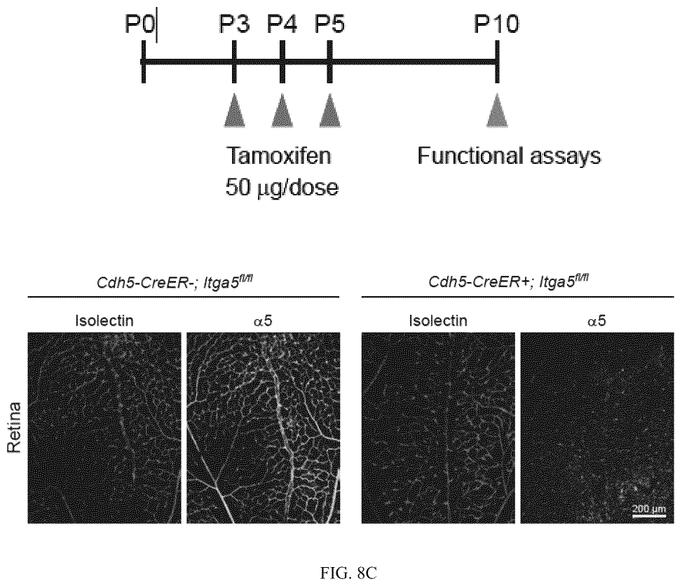

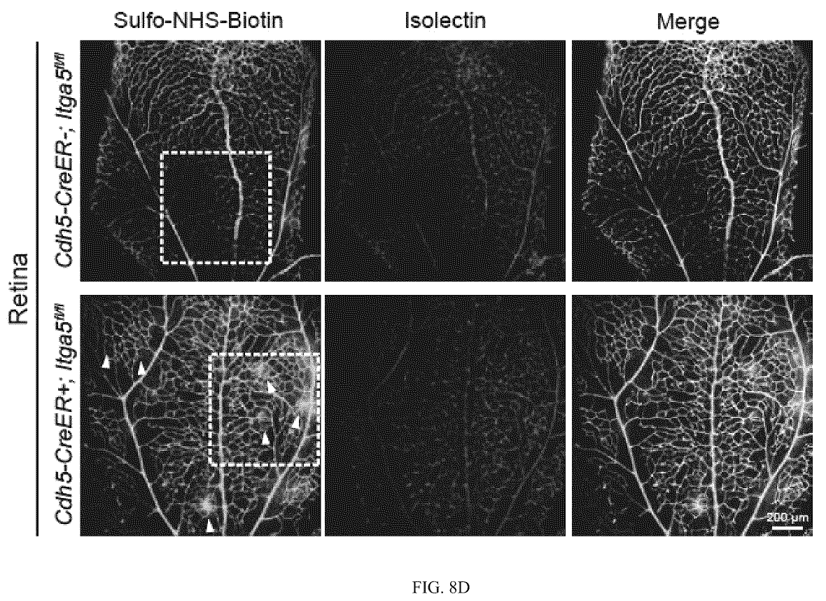

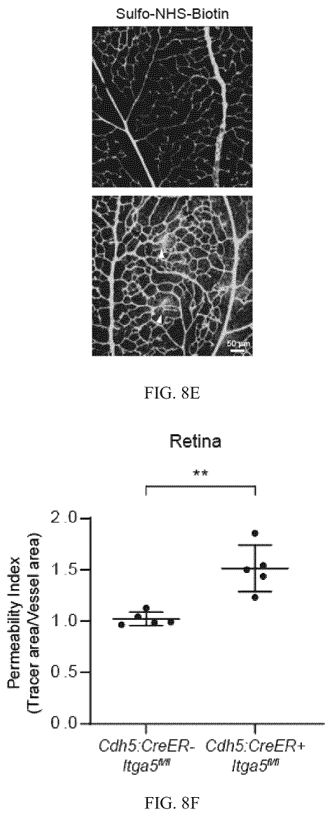

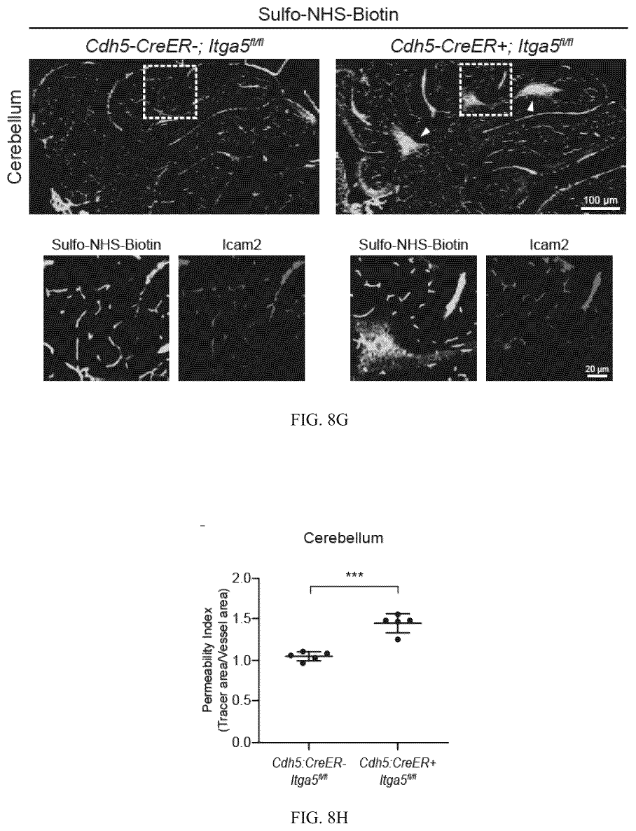

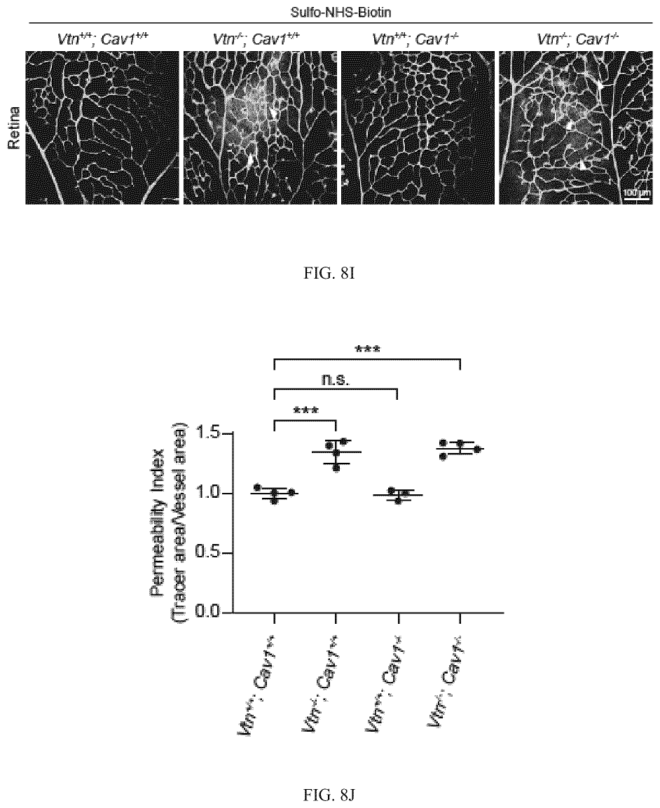

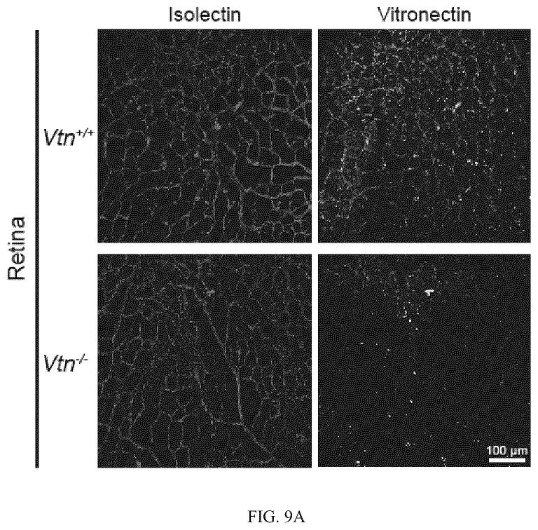

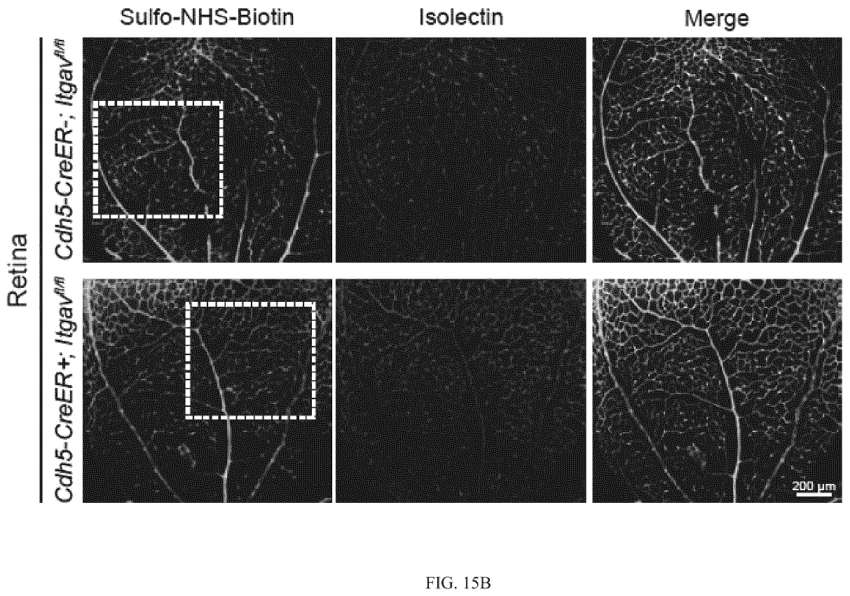

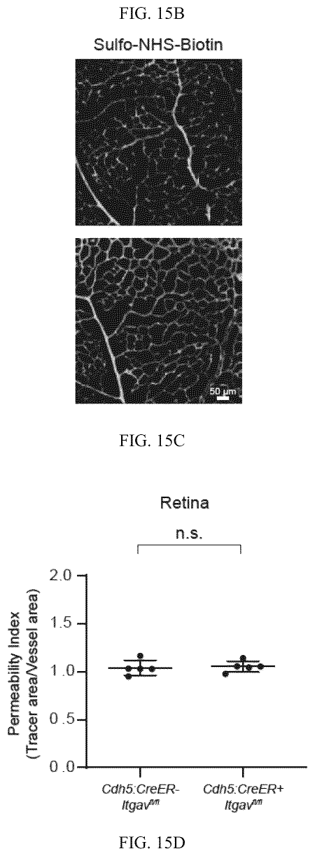

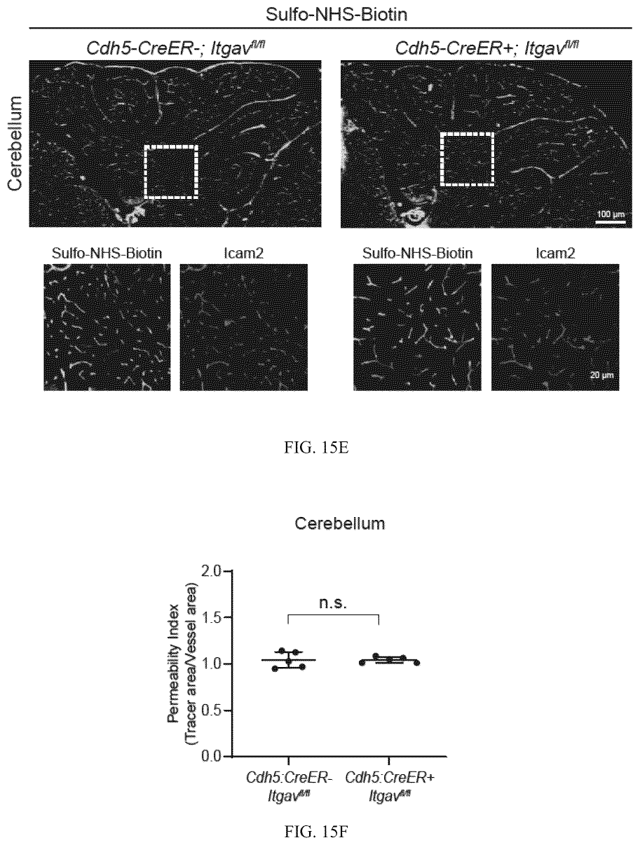

To identify the relevant endothelial integrin receptors in vivo, in situ hybridization was performed to examine the localization of Itga5 and Itgav transcripts in the brain. Single cell RNA-seq data revealed transcripts for both genes in CNS endothelial cells (Vanlandewijck et al., 2018); α5 and αv are also known to co-operate during vascular development (Van Der Flier et al., 2010). RNAscope revealed that although both Itga5 and Itgav mRNA were present in endothelial cells as well as pericytes in the brain (FIGS. 8A, 15A), Itga5 mRNA was predominantly expressed in endothelial cells whereas Itgav mRNA was predominately expressed in pericytes (FIG. 8B). On average, Itga5 transcripts in endothelial cells were 20.3±1.5 and in pericytes it was 5.3±0.43. In contrast, the average Itgav transcripts in endothelial cells was 8.3±0.7 and in pericytes it was 19.5±1.2 (FIG. 8B, Mean±SEM). Thus, Itga5 was predominantly expressed in endothelial cells while Itgav was predominantly in pericytes.

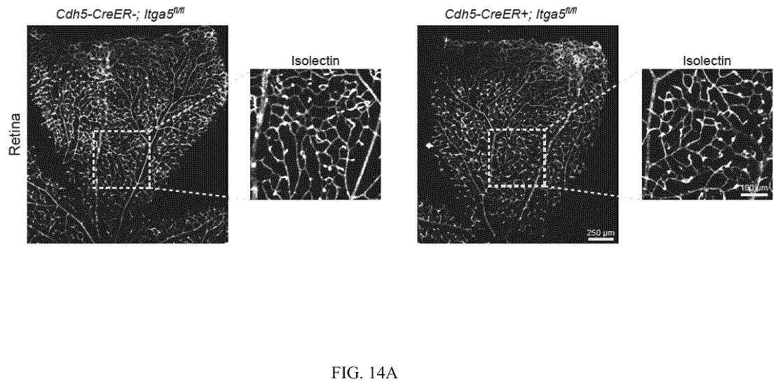

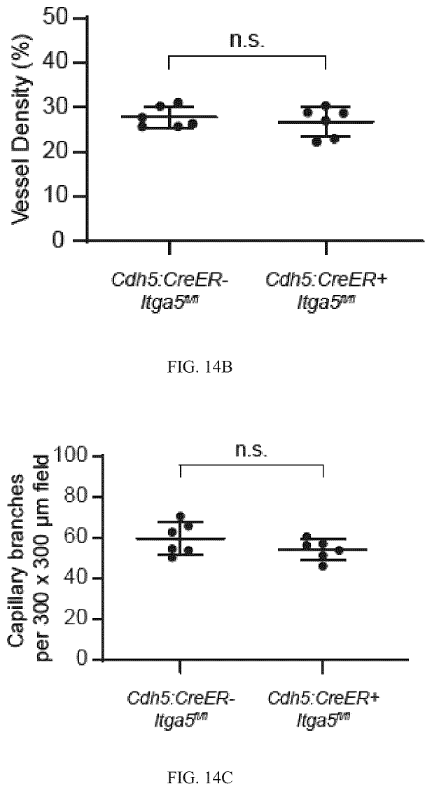

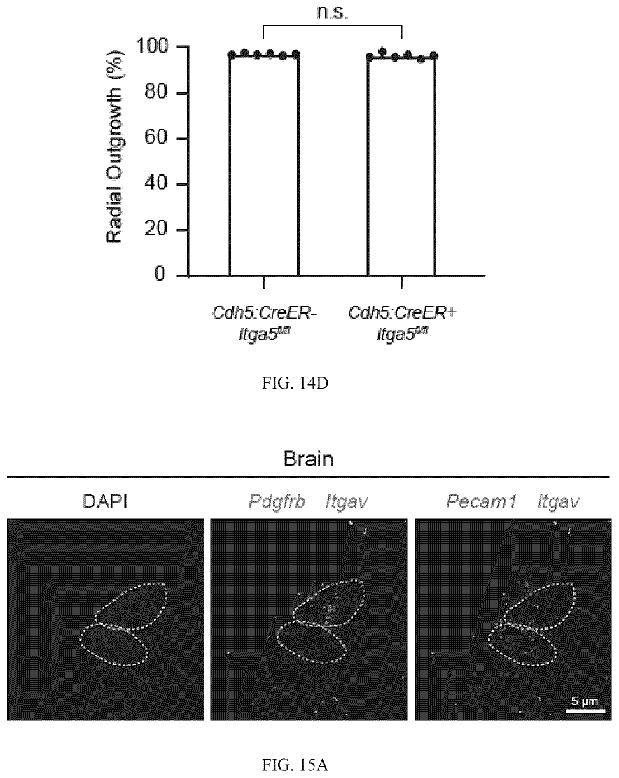

Next, the role of endothelial Itga5 and Itgav in barrier function was determined by ablating these genes acutely and specifically in endothelial cells by crossing Itga5^{fl/fl} or Itgav^{fl/fl} (Van Der Flier et al., 2010) mice with endothelial cell-specific Cdh5-CreER mouse line (Wang et al., 2010). Acute deletion of endothelial Itga5 (FIG. 8C) resulted in a leaky blood-retinal barrier (FIG. 8D) with numerous tracer hotspots spread out in the retina tissue (FIGS. 8E, 8F). Itga5^{fl/fl};Cdh5-CreER mutant mice exhibited normal vascular density and patterning (FIGS. 14A-14D). Similar to Vtn^{-/-} and Vtn^{RGE} mice, Itga5^{fl/fl};Cdh5-CreER mutants also had a leaky BBB in the cerebellum (FIGS. 8G, 8H). In contrast to Itga5, mice lacking endothelial Itgav exhibited normal barrier function. The injected tracer was completely confined to the vasculature in both the retina (FIGS. 15B-15D) and the brain tissue (FIGS. 15E, 15F) indicating intact blood-CNS barriers in Itgav^{fl/fl};Cdh5-CreER mice. These experiments established the RGD-specific integrin receptor, α5 as an essential integrin for barrier function, particularly in the retina and cerebellum. Importantly, mice lacking endothelial integrin α5 receptor fully phenocopy Vtn^{-/-} and Vtn^{RGE} mice. Together with the in vitro results, these data indicated that the engagement of vitronectin-integrin α5 actively inhibited transcytosis in CNS endothelial cells to maintain barrier integrity. Further, caveolae-mediated transcytosis is one pathway that is actively inhibited in CNS endothelial cells by Mfsd2a. To determine whether caveolae-mediated transcytosis is also downstream of vitronectin-integrin signaling, a genetic rescue experiment by crossing Vtn^{-/-} mice with Cav-1^{-/-} mice (31) was performed because caveolin-1 is an obligatory protein required for caveolae formation. Genetic ablation of Cav-1 in Vtn^{-/-} mice did not rescue barrier leakage: similar to Vtn^{-/-} single knockouts, we observed leakage in Vtn^{-/-}; Cav-1^{-/-} double knockout mice as well (FIGS. 8I-8J), indicating that vitronectin-mediated inhibition of transcytosis is caveolae-independent. Consistent with this result, no detectable changes in Mfsd2a localization and expression in the retinas of Vtn^{-/-} mice compared to wildtype littermates was observed. Therefore, vitronectin-integrin α5 mediated regulation of barrier integrity is independent of the caveolae pathway.

### Material and Methods

Mice

All mouse experiments were performed according to institutional and US National Institutes of Health (NIH) guidelines approved by the International Animal Care and Use Committee (IACUC) at Harvard Medical School. Mice were maintained on 12 light/12 dark cycle.

The following mice strains were used: Vtn null mice (JAX: 004371) and Vtn^{RGE/RGE} mice (Wheaton et al., 2016); NG2:DsRed (JAX: 008241), Itga5^{flox} (JAX: 032299), Itgav^{flox} (JAX: 032297). All mice were maintained on C57/BL6J background. All leakage assays were performed at P10, RNAscope was performed at P7. In vivo siRNA experiments were done in 6-week-old mice.

Cre-mediated recombination was induced by intraperitoneal injection of 50 ug of 1 mg/ml Tamoxifen (T5468, Sigma dissolved in peanut oil containing ethanol 1:40 by volume) for 3 consecutive days, P3-P5. All animals were genotyped with allele-specific PCR reactions prior to experiments. Both males and females were used across all experiments and no sex-dependent phenotype was observed.

Across all mouse experiments, mice were genotyped the day of experiments and experiments were not performed genotype-blinded. However, all analyses were blinded. No statistical methods were used to predetermine samples sizes but our sample sizes are consistent with previous publications and standards in the field.

Immunohistochemistry

Enucleated eyes of P10 pups were fixed in 4% ice-cold PFA (EMS, 15713) for 5 minutes at room temperature (RT). Retinas were then dissected in 4% PFA and allowed to fix for 30 minutes at RT (adult retinas were fixed for 1 hour at RT). Retinas were washed in PBS three times for 5 minutes each and incubated in blocking buffer (10% normal donkey serum with 5% bovine serum albumin and 0.5% triton in PBS) for 1 hour at RT. Retinas were then incubated with primary antibodies in blocking buffer overnight at 4° C. The next day retinas were washed again with PBS three times for 5 minutes each and incubated with secondary antibodies in blocking buffer for 1 hour at RT and washed again with PBS. Retinas were then dissected into 4 leaflets and flat mounted on glass slides with vitreal surface in contact with coverslips using Prolong gold Antifade mountant (Thermo Fisher Scientific P36934).

Dissected brains were drop-fixed in 4% PFA overnight at 4° C. The brains were washed with PBS three times 10 minutes each and cryopreserved in 30% sucrose. The brains were bisected along the midline to obtain sagittal sections, before freezing in TissueTek OCT (Sakura). 20 um cryosections obtained on the cryostat were then processed for immunostaining. Brain sections were first permeabilized with 0.2% triton in blocking buffer (10% normal donkey serum with 5% bovine serum albumin in PBS) for 10 minutes at RT. The rest of the staining procedure was similar to the stainings in retina as above, except secondary antibodies were incubated for 45 minutes at RT.

Isolectin GS-IB₄ conjugated to Alexa Fluor 568 (Thermo Fisher Scientific I21412) or Isolectin GS-IB₄ conjugated to Alexa Fluor 647 (Thermo Fisher Scientific I32450) was incubated (1:300) along with primary antibodies to stain vasculature in retinas. Streptavidin conjugated to Alexa Fluor 647 (Thermo Fisher Scientific S32357) was incubated (1:300) along with secondary antibodies to detect the injected Sulfo-NHS-Biotin. The following primary antibodies were used: rabbit α-Vitronectin (Genway Biotech GWB-794F8F, 1:100), goat α-CD31 (PECAM1, R&D Systems AF3628, 1:100), mouse α-Claudin-5 (Thermo Fisher Scientific 352588, 1:200), rabbit α-ZO-1 (Invitrogen 40-2200, 1:200), rat α-CD102 (ICAM2, BD Biosciences 553326, 1:100), rabbit α-ERG1/2/3 (Abcam ab92513, 1:200), rabbit α-Collagen IV (Bio-rad 2150-1470, 1:200), rat α-Perlecan (EMD Millipore, MAB1948P, 1:200), goat α-laminin α4 (R&D systems, AF3837, 1:100) rat α-CD49e (α5, BD Biosciences 553319, 1:100). All corresponding secondary antibodies were used at 1:300 obtained from Jackson Immunoresearch Laboratories.

Fluorescent In Situ Hybridization

Brains and lungs were dissected from 1-week old wildtype animals, flash frozen in liquid nitrogen and cryosectioned to obtain 20 um sections. RNAscope was performed according to manufacturer's instructions (ACD Bio) with the following probes: Vtn (443601), Pdgfrb (411381-C3), PECAM1 (316721-C2), Itga5 (575741) Itgav (513901). To perform immunostaining post RNAscope, slides were briefly rinsed in PBS and incubated with blocking buffer (3% normal donkey serum with 3% bovine serum albumin in PBS) for 1 hour at RT. The rest of the steps for staining are similar to the staining protocol mentioned above under immunohistochemistry. DAPI (Thermo Fisher Scientific 62247) was added at 1:5000 dilution in the last PBS wash before mounting slides.

Barrier Permeability Assays

P10 pups were briefly anaesthetized with 3% isoflurane. Eyelid of one eye was cut off and tracer was injected retro-orbitally (Yardeni et al., 2011) with a 30-gauge needle. Tracer was allowed to circulate for 5 minutes followed by dissection of contralateral retina and brain, and processed for immunohistochemistry.

Tracers include EZ-Link Sulfo-NHS-LC-Biotin (Thermo Fisher Scientific, 21335, 0.44 kDa) injected at 0.5 mg/gm body weight and 10 kDa Dextran conjugated Alexa 488 (Thermo Fisher Scientific, D22910) injected at 0.2 mg/gm body weight. Tracers were made fresh in PBS, dissolved in 5 ul of PBS/gm body weight.

In Vivo siRNA Experiments

250 nmol of Ambion's HPCL-IVR (HPLC grade, in vivo ready) pre-designed siRNAs targeting mouse vitronectin (Catalog No. 4457308, siRNA IDs s76001, s76002) and a control siRNA (Catalog No. 4457289) were ordered from ThermoFisher. Invivofectamine 3.0 (Thermo Fisher, IVF3001) was used to form siRNA complexes, as per manufacturer's instructions for systemic delivery into mice. siRNA duplexes were resuspended in DNase/RNase free water (Thermo Fisher, 10977) at 4.8 mg/ml, the complexation in Invivofectamine 3.0 was followed as per protocol. 100 ul of siRNA complex was injected into 6-week-old mice (˜20 gm) to result in 1 mg/kg dosing. siRNA complexes were injected into circulation through tail vein on 2 consecutive days. Subsequent experiments included confirmation of knock-down and leakage assays which were performed either 24 hours (day 3) or 72 hours (day 5) post last siRNA injection. Confirmation of vitronectin knock-down was determined by ELISA on plasma isolated from mice. For leakage assays, Sulfo-NHS-biotin was injected (0.5 mg/gm body weight) into circulation via the tail-vein. Across all mice, retinas from left eyes were used for leakage analyses.

Plasma Isolation

On the day of experiments, mice were briefly anaesthetized with 3% isoflurane and heparinized capillary tubes 1.1 mm×7.5 mm (Thomas Scientific, 44B508) were used to collect blood from the retro-orbital sinus of the right eye. Collected blood was transferred to Eppendorf tubes containing 10 ul 0.5 M EDTA, pH 8.0 (Thermo Fisher, 15575020) and vortexed to prevent blood from coagulating. Isolated blood samples were centrifuged for 15 minutes at 2000×g at 4° C. and the supernatant was collected. Knockdown of vitronectin in plasma samples was confirmed by an ELISA kit for vitronectin (Molecular Innovations, MVNKT-TOT).

Transmission Electron Microscopy

HRP (Thermo Fisher Scientific, 31491), prepared fresh before each experiment was injected into the retro-orbital sinus of P10 pups briefly anaesthetized with isoflurane. HRP was injected at 0.5 mg/gm body weight, dissolved in 5 ul of PBS/body weight. Tracer was circulated for 10 minutes followed by enucleation of the contralateral eye and retina dissection in 4% PFA made in 0.1 M sodium cacodylate buffer (EMS, 11653). The brain was also dissected and both the tissues were first fixed in 5% glutaraldehyde (EMS, 16200) and 4% PFA mixture made in 0.1 M sodium cacodylate for 1 hour at RT. The brains and retinas were then fixed overnight at 4° C. in 4% PFA in 0.1 M sodium cacodylate.

Following fixation both tissues were washed with 0.1 M sodium cacodylate buffer three times, 5 minutes each. 100 um sagittal sections of the brain and the whole retina were then incubated with fresh 0.5 mg/ml DAB (Sigma, D5905) for 25 min at RT. DAB was prepared in 0.1 M sodium cacodylate containing 0.05 M Tris-HCl and 0.01% hydrogen peroxide. Leaky areas within the retina and cerebellum were further microdissected, cut into 80 nm ultrathin sections and further processed for EM analysis as described previously (Chow and Gu, 2017).

Western Blotting

Retinas were dissected in PBS containing protease inhibitors and phosphatase inhibitors (Thermo Fisher Scientific, 87786 and 78420 respectively). Dissected retinas were further minced with a razor blade and allowed to lyse on ice for 30 minutes in lysis buffer (50 mM Tris-HCl, pH 7.4, 150 mM NaCl, 1% triton-X 100, 0.1% SDS supplemented with protease and phosphatase inhibitors). Lysed retinas were centrifuged for 15 minutes at 4° C., the supernatant was collected and BCA assay (Thermo Fisher Scientific, 23225) was performed according to manufacturer's instructions to determine total protein concentrations. Samples were then analyzed for various proteins using SDS-PAGE and Western Blotting. The following antibodies were used: Pdgfrβ (Cell Signaling Technologies 3169, 1:500), Fibronectin (Abcam ab2413, 1:200)

Cell Culture Media, Growing Conditions and Coverslip Coating

Mouse primary brain microvascular endothelial cells (Cell Biologics, C57-6023) were maintained in a complete mouse endothelial cell medium (Cell Biologics, M1168) supplied with VEGF, ECGS, Heparin, EGF, and FBS, according to the manufacture's instruction. To functionalize the surface of cover glasses, the cover glasses were cleaned (Thomas Scientific, 1217N79) with air plasma (Harrick Plasma) for 15 minutes. For surface functionalization with single matrix proteins, the cleaned cover glasses were incubated with 50 μg/mL mouse collagen IV (Corning, 354233) in water, 50 μg/mL laminin 511 (Sigma-Aldrich, CC160) in PBS, or 50 μg/mL multimeric vitronectin (Molecular Innovations, HVN-U) in PBS, for 1 h at 37° C. For surface functionalization with collagen IV plus laminin, the collagen IV-functionalized cover glasses were air dried and then incubated with 50 μg/mL laminin 511 in PBS for 1 hour at 37° C. For surface functionalization with Collagen IV plus laminin and vitronectin, the collagen IV-functionalized cover glasses were air dried and then incubated with 50 μg/mL laminin 511 and vitronectin in PBS for 1 hour at 37° C. The functionalized cover glasses were washed with cell culture media 3 times before cell plating. Cells were gently detached from tissue culture dishes using enzyme-free cell dissociation buffer (Gibco, 13151014) and then seeded on the functionalized cover glasses for at least 4 hours before next treatment.

Imaging Focal Adhesions in Cell Culture

To image integrin α5-mediated adhesion, the cells were snap chilled in ice-cold HBSS, which is 1× HBSS (Gibco, 24020117) buffered with 10 mM HEPES (Gibco, 15630106). The cells were then incubated with primary antibody rat α-CD49e (α5, BD Biosciences 553319, 1:100) in HHBSS for 30 minutes at 4° C. and washed with ice-cold HHBSS 3 times for 5 minutes each. The cells were fixed in 4% ice-cold PFA in BPS for 15 minutes at RT and permeabilized with 0.1% triton-X in PBS for 15 minutes at RT. The cells were then washed with PBS 3 times for 5 minutes each and incubated in blocking buffer (5% bovine serum albumin in PBS) for 1 hour at RT. To simultaneously image paxillin, vinculin or pFAK with integrin α5, the blocked samples were incubated with corresponding primary antibody in blocking buffer for 2 hours at RT and washed with PBS 3 times for 5 minutes each. Primary antibodies include mouse α-paxillin (BD Biosciences 610051, 1:100), mouse α-vinculin (Sigma-Aldrich V9131, 1:100) and rabbit α-FAK (phosphor Y397 from Abcam ab81298, 1:100). The samples were finally incubated with corresponding secondary antibodies at 1:500, phalloidin-iFluor 488 (Abcam, ab176753), and nuclear stain which is 1 μg/mL of either Hoechst 33342 (Thermo Scientific, 62249) or Ethidium Homodimer-1 (Thermo Scientific, E1169) in blocking buffer for 1 hour at RT and washed with PBS 5 times for 5 minutes each before imaging.

In Vitro shRNA and Endocytosis Experiments

The DNA sequences encoding shRNA1 and 2 for integrin α5 knockdown are “CCCAGCAGGGAG-TCGTATTTACTCGAGTAAATACGACTCCCTGCTGGG” (SEQ ID NO: 3) and “ATCAACTTGGAACCATAATTACTCGAGTA-ATTATGGTTCCAAGTTGAT” (SEQ ID NO: 4), respectively. The DNA sequence encoding negative control or scramble shRNA is “CCTAAGGTTAAGTCGCCCTCGCTCGAGCGAGGGCGACTTAACCTTAGG” (SEQ ID NO: 13), which is designed based on previously published scramble shRNA plasmid (addgene plasmid #1864). These DNA fragments were cloned into pLKO.1 (Addgene plasmid #10878) for RNA transcription in cells. The DNA encoding EBFP2 was cloned into the pLKO.1 vector in substitution of the sequence encoding neomycin resistant protein to report the transcription of shRNA. To package lentivirus for RNAi, HEK293FT cells (Invitrogen, R70007) in 35-mm tissue culture dishes were transfected with 1.5 μg pLKO.1 shRNA plasmid, 0.8 μg psPAX2 (Addgene plasmid #12260), 0.7 μg pMD2.G (Addgene plasmid #12259), and 9 μL Turbofect transfection reagent (Thermo Scientific, R0533) in 2 mL of Opti-MEM (Gibco, 31985062) for 12 hours. The viruses were produced in DMEM supplied with 10% FBS and 110 mg/mL sodium pyruvate (Gibco) and harvested 24 hours after transfection. The mouse primary brain endothelial cells were infected with lentivirus and used for experiments 3 days after infection.

To test endocytosis, cells were washed with HHBSS 3 times and starved in HHBSS for 1 hour at 37° C. The cells were then incubated with 5 μg/mL of FM™ 1-43FX (Thermo Fisher Scientific, F35355) in HHBSS for 15 minutes at 37° C. and then quickly washed with HHBSS 5 times. The cells were immediately fixed in 4% PFA in PBS for 15 minutes at RT and washed with PBS 3 times before imaging.

Imaging

Leakage assays in brain tissue were imaged on Olympus VS120 slide scanner. Rest of the fluorescent imaging was acquired on Leica TCS SP8 confocal. Z-stacks were obtained and all maximum-intensity projections are shown in all figures. TEM images were acquired on a 1200EX electron microscope (JEOL) equipped with a 2 k CCD digital camera (AMT). All images were processed using FIJI. Cell culture imaging was acquired on an epi-fluorescence Leica DMI 6000B microscope.

### Quantification and Statistical Analysis

Except for scatter plots, all graphs represent mean±S.D. All graphs show individual data points throughout the paper. For each graph, the sample size and statistical test used are described in the corresponding figure legend. No method was used to predetermine whether the data met assumptions of the statistical approach. For mouse experiments n is the number of mice whereas for in vitro experiments n is the number of cells.

Fluorescent In Situ Hybridization

To determine percentage of pericytes positive for Vtn mRNA transcripts, PECAM1 immunostaining was first used to determine if cells positive for Pdgfrb transcripts were pericytes. Vessels containing only a single nucleus and ≤5 μm in width as shown in FIG. 1C were identified as capillaries and Pdgfrb+ cells in close-contact or abutting the vessel were identified as pericytes (FIG. 1C).

Scatter plots for Pdgfrb vs Vtn mRNA puncta was obtained by first identifying nuclei using the DAPI channel. DAPI containing nuclei were first segmented using FIJI and segmented regions were recorded in ROI manager. Fluorescent images of Pdgfrb and Vtn were thresholded using ‘Otsu’ method. Pdgfrb and Vtn puncta numbers in each of the segmented regions were obtained from the thresholded images using ‘Find Maxima’ with tolerance ≥30. Puncta numbers obtained for both transcripts in a given cell were plotted as X-Y scatter.

Scatter plot for Itga5 or Itgav mRNA transcripts in endothelial cells vs pericytes was obtained in a similar manner. However, the DAPI nuclei segmented were first assigned as endothelial cells or pericytes using Pecam1 and Pdgfrb mRNA respectively.

Leakage Assays

Leakage was quantified as previously described (Andreone et al., 2017; Chow and Gu, 2017). For retinas, 770 μm×770 μm areas across all 4 leaflets were first maximum intensity Z-projected, background subtracted, thresholded to obtain area of vessel (isolectin for retinas and ICAM2 for brain sections) and tracer (Sulfo-NHS-Biotin). ‘Default’ threshold method was used for thresholding isolectin signal while ‘Li’ thresholding was used for streptavidin (tracer) signal. Permeability index was defined as the ratio of area of tracer to area of vessel. Permeability index value of 1 implies tracer confined within vessels while values >1 indicate tracer extravasation from vessels. For leakage analysis of brain tissues, 6-8 sections were quantified per animal. Average of values across these regions/sections for a given animal was treated as one biological sample.

ELISA for Vitronectin Protein Expression Levels

ELISA analyses were performed using Arigo Biolaboratories free ELISA calculator at arigobio.com/elisa-analysis. Standard curve was generated by fitting the data to a 4-parameter logistic (4PL) curve. Our preliminary ELISA runs determined that plasma samples diluted 1:500 were usually in the range of the standard curve.

Vesicular Density From EM Data

For both retina and cerebellum, 15-20 vessels per animal were imaged under an electron microscope. Low magnification images encompassing complete blood vessel was first acquired using which the entire cytoplasmic area of each vessel was determined. For every blood vessel, tracer-filled vesicles were manually counted and then normalized to cytoplasmic area of the vessel (excluding area of nucleus) to obtain vesicular density. Average of vesicular density across all vessels of a given animal was considered one biological sample.

Vessel Density, Branching, Radial Outgrowth

Tilescan images to capture entire leaflets of retinas were acquired and these parameters were obtained for each leaflet. The average of all 4 leaflets was taken to be the measurement for a given animal. For vessel density measurements, isolectin stained vessel images were auto-thresholded to determine vessel area. Ratio of vessel area was normalized to total leaflet area to obtain vessel density measurements. For capillary branches, capillary branch points were counted manually in multiple 300 μm×300 μm regions per leaflet, and the average across these regions was obtained for each animal. For P10 retinas, 300 μm×300 μm regions were ideal as it allowed for the exclusion of arteries and veins. For radial outgrowth, vessel images were first auto-thresholded, distance from the optic nerve head to the tip of the retina was measured and this was normalized to distance from optic nerve head to edge of retinal tissue.

Pericyte Density and Coverage

Pericyte density and coverage analyses were performed as described previously (Chow and Gu, 2017). 770 μm×770 μm areas across all 4 leaflets were first maximum intensity Z-projected and background subtracted. Erg and isolectin were auto-thresholded across all images and genotypes. For pericyte density measurements, DsRed signal was thresholded using ‘Otsu’ method to identify only pericyte cell bodies. The number of DsRed+ cell bodies was normalized to endothelial cell nuclei count to determine pericyte density. For pericyte coverage measurements, DsRed signal was thresholded using ‘Li’ method to allow detection of pericyte cell bodies as well as processes. Area of DsRed obtained was then normalized to area of thresholded isolectin to determine pericyte coverage.

Astrocyte Endfeet Coverage From EM Data

Low magnification EM images were acquired to capture entire cross-section of vessels. The vascular basement membrane was first identified in each vessel (encompassing endothelial cells and pericytes) and perimeter of the cross-section was first obtained. Astrocyte endfeet coverage was defined as vessel perimeter in contact with astrocyte endfeet normalized to total perimeter of the vessel cross-section. Average of percent coverage of all vessels from one animal was reported as the percent coverage for that mouse.

Western Blots

All western blots were quantified using the gel analyzer tool in Fiji. Sample values were normalized to GAPDH values from corresponding lanes to account for differences in protein amounts across wells. The ratio of band intensity values of lysates from Vtn^{-/-} mice to wildtype mice was then obtained to plot fold-change for mutant mice.

Focal Adhesions and Dye Uptake Assays

All images were processed using ImageJ Fiji. To identify integrin α5-mediated adhesions, the images were first background subtracted and thresholded. The particle analysis tool in Fiji was then used to quantify the number of adhesions in all imaging channels. The bright endocytic vesicles were identified by threshold tool and the ratio (%) of total fluorescence intensity in endocytic vesicles to the whole cell fluorescence intensity was measured to quantify endocytosis.

Statistical Analysis

All statistical analyses were performed using Prism 9 GraphPad software. For comparison between two groups, unpaired two-tailed Student's t-test was used. For comparison across multiple groups, one-way ANOVA with Tukey's post-hoc test was used.

### Example II. FAK Inhibitor Maintains Blood-CNS Barrier Integrity in Vtn^{-/-} Mice

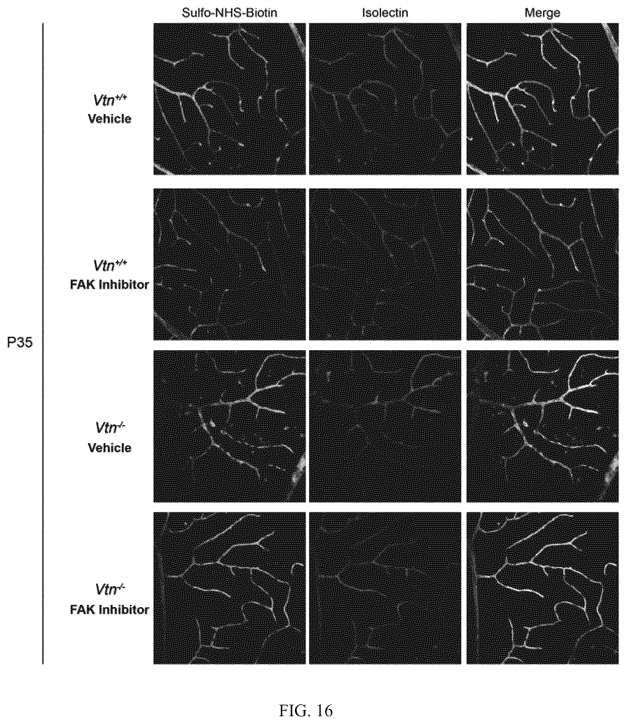

PF-562, 271 is a focal adhesion kinase inhibitor developed by Pfizer. It is an ATP-competitive, reversible inhibitor of FAK, and is10-fold less potent toward Pyk2 and >100-fold selectivity over several other kinases. PF-562, 271 can be absorbed in mice within 30 minutes to 2 hours post administration. PF-562, 271 has shown anti-angiogenic effects at high doses. It is currently in clinical trials now, being tested on patients with cancers including glioblastoma. To test the effect of PF-562, 271 on blood-CNS integrity, 1 mg/ml PF-562, 271 was dissolved in 30% 2-hydroxypropyl-β-cyclodextrin and 2.5% dextrose. It is prepared freshly prior to administration for all the time points tested. PF-562, 271 was delivered to Vtn^{+/+} or Vtn^{-/-} mice by IP administration on at 20 mg/kg on day 1 and day 2. Mice were sacrificed on day 3 and leakage assay was performed. The results showed that administration of Pf-562,271 was capable of decrease blood-CNS permeability in Vtn^{-/-} mice (FIG. 16).

### REFERENCES

Andreone, B. J., Chow, B. W., Tata, A., Lacoste, B., Ben-Zvi, A., Bullock, K., Deik, A. A., Ginty, D. D., Clish, C. B., and Gu, C. (2017). Blood-Brain Barrier Permeability Is Regulated by Lipid Transport-Dependent Suppression of Caveolae-Mediated Transcytosis. Neuron 94, 581-594.e5.

Armulik, A., Genové, G., Mäe, M., Nisancioglu, M. H., Wallgard, E., Niaudet, C., He, L., Norlin, J., Lindblom, P., Strittmatter, K., et al. (2010). Pericytes regulate the blood-brain barrier. Nature 468, 557-561.

Bauer, J. S., Schreiner, C. L., Giancotti, F. G., Ruoslahti, E., and Juliano, R. L. (1992). Motility of fibronectin receptor-deficient cells on fibronectin and vitronectin: Collaborative interactions among integrins. J. Cell Biol. 116, 477-487.

Bell, R. D., Winkler, E. A., Sagare, A. P., Singh, I., LaRue, B., Deane, R., and Zlokovic, B. V. (2010). Pericytes Control Key Neurovascular Functions and Neuronal Phenotype in the Adult Brain and during Brain Aging. Neuron 68, 409-427.

Ben-Zvi, A., Lacoste, B., Kur, E., Andreone, B. J., Mayshar, Y., Yan, H., and Gu, C. (2014). Mfsd2a is critical for the formation and function of the blood-brain barrier. Nature 509, 507-511.

Cherny, R. C., Honan, M. A., and Thiagarajan, P. (1993). Site-directed mutagenesis of the arginine-glycine-aspartic acid in vitronectin abolishes cell adhesion. J. Biol. Chem. 268, 9725-9729.

Chow, B. W., and Gu, C. (2017). Gradual Suppression of Transcytosis Governs Functional Blood-Retinal Barrier Formation. Neuron 93, 1325-1333.e3.

Daneman, R., Agalliu, D., Zhou, L., Kuhnert, F., Kuo, C. J., and Barres, B. A. (2009). Wnt/β-catenin signaling is required for CNS, but not non-CNS, angiogenesis. Proc. Natl. Acad. Sci. U. S. A. 106, 641-646.

Daneman, R., Zhou, L., Kebede, A. A., and Barres, B. A. (2010). Pericytes are required for bloodĝ€″ brain barrier integrity during embryogenesis. Nature 468, 562-566.

Diz-Muñoz, A., Fletcher, D. A., and Weiner, O. D. (2013). Use the force: Membrane tension as an organizer of cell shape and motility. Trends Cell Biol. 23, 47-53.

Eilken, H. M., Diéguez-Hurtado, R., Schmidt, I., Nakayama, M., Jeong, H. W., Arf, H., Adams, S., Ferrara, N., and Adams, R. H. (2017). Pericytes regulate VEGF-induced endothelial sprouting through VEGFR1. Nat. Commun. 8.

Van Der Flier, A., Badu-Nkansah, K., Whittaker, C. A., Crowley, D., Bronson, R. T., Lacy-Hulbert, A., and Hynes, R. O. (2010). Endothelial α5 and αv integrins cooperate in remodeling of the vasculature during development. Development 137, 2439-2449.

Frank, R. N., Dutta, S., and Mancini, M. A. (1987). Pericyte coverage is greater in the retinal than in the cerebral capillaries of the rat. Investig. Ophthalmol. Vis. Sci. 28, 1086-1091.

He, L., Vanlandewijck, M., Raschperger, E., Andaloussi Maë, M., Jung, B., Lebouvier, T., Ando, K., Hofmann, J., Keller, A., and Betsholtz, C. (2016). Analysis of the brain mural cell transcriptome. Sci. Rep. 6, 1-13.

Heithoff, B. P., George, K. K., Phares, A. N., Zuidhoek, I. A., Munoz-Ballester, C., and Robel, S. (2021). Astrocytes are necessary for blood-brain barrier maintenance in the adult mouse brain. Glia 69, 436-472.

Hynes, R. O. (2002). Integrins: bidirectional, allosteric signaling machines. Cell 110, 673-687.

Izawa, Y., Gu, Y. H., Osada, T., Kanazawa, M., Hawkins, B. T., Koziol, J. A., Papayannopoulou, T., Spatz, M., and del Zoppo, G. J. (2018). B1-Integrin-Matrix Interactions Modulate Cerebral Microvessel Endothelial Cell Tight Junction Expression and Permeability. J. Cereb. Blood Flow Metab. 38, 641-658.

Kant, R., Halder, S. K., Bix, G. J., and Milner, R. (2019). Absence of endothelial α5β1 integrin triggers early onset of experimental autoimmune encephalomyelitis due to reduced vascular remodeling and compromised vascular integrity. Acta Neuropathol. Commun. 7, 11.

Katsumi, A., Orr, A. W., Tzima, E., and Schwartz, M. A. (2004). Integrins in Mechanotransduction. J. Biol. Chem. 279, 12001-12004.

Langen, U. H., Ayloo, S., and Gu, C. (2019). Development and cell biology of the blood-brain barrier. Annu. Rev. Cell Dev. Biol. 35, 591-613.

Leavesley, D. I., Kashyap, A. S., Croll, T., Sivaramakrishnan, M., Shokoohmand, A., Hollier, B. G., and Upton, Z. (2013). Vitronectin—master controller or micromanager? IUBMB Life 65, 807-818.

Liebner, S., Corada, M., Bangsow, T., Babbage, J., Taddei, A., Czupalla, C. J., Reis, M., Felici, A., Wolburg, H., Fruttiger, M., et al. (2008). Wnt/β-catenin signaling controls development of the blood-brain barrier. J. Cell Biol. 183, 409-417.

Macosko, E. Z., Basu, A., Satija, R., Nemesh, J., Shekhar, K., Goldman, M., Tirosh, I., Bialas, A. R., Kamitaki, N., Martersteck, E. M., et al. (2015). Highly parallel genome-wide expression profiling of individual cells using nanoliter droplets. Cell 161, 1202-1214.

La Manno, G., Siletti, K., Furlan, A., Gyllborg, D., Vinsland, E., Mossi Albiach, A., Mattsson Langseth, C., Khven, I., Lederer, A. R., Dratva, L. M., et al. (2021). Molecular architecture of the developing mouse brain. Nature 596, 92-96.

Orlando, R. A., and Cheresh, D. A. (1991). Arginine-glycine-aspartic acid binding leading to molecular stabilization between integrin α(v)β3 and its ligand. J. Biol. Chem. 266, 19543-19550.

Park, D. Y., Lee, J., Kim, J., Kim, K., Hong, S., Han, S., Kubota, Y., Augustin, H. G., Ding, L., Kim, J. W., et al. (2017). Plastic roles of pericytes in the blood-retinal barrier. Nat. Commun. 8, 1-16.

Preissner, K. T. (1991). Structure and Biological Role of Vitronectin. Annu. Rev. Cell Biol. 7, 275-310.

Preissner, K. T., and Reuning, U. (2011). Vitronectin in vascular context: Facets of a multitalented matricellular protein. Semin. Thromb. Hemost. 37, 408-424.

Reese, T. S., and Karnovsky, M. J. (1967). Fine structural localization of a blood-brain barrier to exogenous peroxidase. J. Cell Biol. 34, 207-217.

Ross, T. D., Coon, B. G., Yun, S., Baeyens, N., Tanaka, K., Ouyang, M., and Schwartz, M. A. (2013). Integrins in mechanotransduction. Curr. Opin. Cell Biol. 25, 613-618.

Sheetz, M. P., and Dai, J. (1996). Modulation of membrane dynamics and cell motility by membrane tension. Trends Cell Biol. 6, 85-89.

Shepro, D., and Morel, N. M. L. (1993). Pericyte physiology. FASEB J. 7, 1031-1038.

Song, E., Lee, S. K., Wang, J., Ince, N., Ouyang, N., Min, J., Chen, J., Shankar, P., and Lieberman, J. (2003). RNA interference targeting Fas protects mice from fulminant hepatitis. Nat. Med. 9, 347-351.

Stenman, J. M., Rajagopal, J., Carroll, T. J., Ishibashi, M., McMahon, J., and McMahon, A. P. (2008). Canonical Wnt signaling regulates organ-specific assembly and differentiation of CNS vasculature. Science 322, 1247-1250.

Stewart, P. A., and Wiley, M. J. (1981). Developing nervous tissue induces formation of blood-brain barrier characteristics in invading endothelial cells: A study using quail-chick transplantation chimeras. Dev. Biol. 84, 183-192.

Strasser, G. A., Kaminker, J. S., and Tessier-Lavigne, M. (2010). Microarray analysis of retinal endothelial tip cells identifies CXCR4 as a mediator of tip cell morphology and branching. Blood 115, 5102-5110.

Thomsen, M. S., Routhe, L. J., and Moos, T. (2017). The vascular basement membrane in the healthy and pathological brain. J. Cereb. Blood Flow Metab. 37, 3300-3317.

Thottacherry, J. J., Kosmalska, A. J., Kumar, A., Vishen, A. S., Elosegui-Artola, A., Pradhan, S., Sharma, S., Singh, P. P., Guadamillas, M. C., Chaudhary, N., et al. (2018). Mechanochemical feedback control of dynamin independent endocytosis modulates membrane tension in adherent cells. Nat. Commun. 9.

Vanlandewijck, M., He, L., Mäe, M. A., Andrae, J., Ando, K., Del Gaudio, F., Nahar, K., Lebouvier, T., Laviña, B., Gouveia, L., et al. (2018). A molecular atlas of cell types and zonation in the brain vasculature. Nature 554, 475-480.

Wang, J., and Milner, R. (2006). Fibronectin promotes brain capillary endothelial cell survival and proliferation through alpha5beta1 and alphavbeta3 integrins via MAP kinase signalling. J. Neurochem. 96, 148-159.

Wang, L., Zhang, X., Liu, X., Feng, G., Fu, Y., Milner, R., and Li, L. (2019a). Overexpression of α5β1 integrin and angiopoietin-1 co-operatively promote blood-brain barrier integrity and angiogenesis following ischemic stroke. Exp. Neurol. 321.

Wang, Y., Nakayama, M., Pitulescu, M. E., Schmidt, T. S., Bochenek, M. L., Sakakibara, A., Adams, S., Davy, A., Deutsch, U., Lüthi, U., et al. (2010). Ephrin-B2 controls VEGF-induced angiogenesis and lymphangiogenesis. Nature 465, 483-486.

Wang, Y., Rattner, A., Zhou, Y., Williams, J., Smallwood, P. M., and Nathans, J. (2012). Norrin/Frizzled4 signaling in retinal vascular development and blood brain barrier plasticity. Cell 151, 1332-1344.

Wang, Y., Sabbagh, M. F., Gu, X., Rattner, A., Williams, J., and Nathans, J. (2019b). Beta-catenin signaling regulates barrier-specific gene expression in circumventricular organ and ocular vasculatures. Elife 8, 1-36.

Wheaton, A. K., Velikoff, M., Agarwal, M., Loo, T. T., Horowitz, J. C., Sisson, T. H., and Kim, K. K. (2016). The vitronectin RGD motif regulates TGF-β-induced alveolar epithelial cell apoptosis. Am. J. Physiol.—Lung Cell. Mol. Physiol. 310, L1206-L1217.

Wrobel, L., Siddiqi, F. H., and Rubinsztein, D. C. (2021). Transient siRNA-mediated protein knockdown in mouse followed by feeding/starving cycle and liver tissue analysis. STAR Protoc. 2, 100500.

Yardeni, T., Eckhaus, M., and Morris, H. D. (2011). Retro-orbital_injections_in_mice lab Animal 2011.pdf. Lab Anim 40, 155-160.

Zender, L., Hütker, S., Liedtke, C., Tillmann, H. L., Zender, S., Mundt, B., Waltemathe, M., Gösling, T., Flemming, P., Malek, N. P., et al. (2003). Caspase 8 small interfering RNA prevents acute liver failure in mice. Proc. Natl. Acad. Sci. U. S. A. 100, 7797-7802.

Zhang, Z., Morla, A. O., Vuori, K., Bauer, J. S., Juliano, R. L., and Ruoslahti, E. (1993). The αvβ1 integrin functions as a fibronectin receptor but does not support fibronectin matrix assembly and cell migration on fibronectin. J. Cell Biol. 122, 235-242.

Zhao, Z., Nelson, A. R., Betsholtz, C., and Zlokovic, B. V. (2015). Establishment and Dysfunction of the Blood-Brain Barrier. Cell 163, 1064-1078.

Zheng, X., Saunders, T. L., Camper, S. A., Samuelson, L. C., and Ginsburg, D. (1995). Vitronectin is not essential for normal mammalian development and fertility. Proc. Natl. Acad. Sci. U. S. A. 92(26): 12426-30.

Del Zoppo, G. J., and Milner, R. (2006). Integrin-matrix interactions in the cerebral microvasculature. Arterioscler. Thromb. Vasc. Biol. 26, 1966-1975.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

1. A method for increasing Blood-Central Nervous System (Blood-CNS) Barrier permeability to treat a disease in a subject, the method comprising administering to the subject an inhibitor of the vitronectin-integrin signaling at the Blood-CNS Barrier.

2. The method of claim 1, wherein the blood-CNS barrier is the blood-brain barrier, or blood-retina barrier.

3.-6. (canceled)

7. The method of claim 1, wherein the integrin is integrin α5.

8. The method of claim 1, wherein the inhibitor of the vitronectin-integrin signaling is a vitronectin inhibitor.

9. (canceled)

10. The method of claim 8, wherein the vitronectin inhibitor is an inhibitory nucleic acid targeting VTN.

11. (canceled)

12. The method of claim 10, wherein the inhibitory nucleic acid targeting VTN is a siRNA comprising an antisense strand comprising the nucleic acid sequence of SEQ ID NOs: 7 or 8.

13.-14. (canceled)

15. The method of claim 8, wherein the vitronectin inhibitor is an antibody, an antibody variant or an antigen-binding fragment targeting vitronectin.

16. The method of claim 1, wherein the inhibitor of the vitronectin-integrin signaling is an integrin α5 inhibitor.

17.-18. (canceled)

19. The method of claim 16, wherein the integrin α5 inhibitor is an inhibitory nucleic acid targeting ITGA5.

20. (canceled)

21. The method of claim 19, wherein the inhibitor nucleic acid targeting ITGA5 is a shRNA comprising the nucleic acid sequence of SEQ ID NO: 3 or 4.

22.-23. (canceled)

24. The method of claim 16, wherein the integrin α5 inhibitor is an antibody targeting integrin α5.

25. The method of claim 16, wherein the integrin α5 inhibitor is a peptide containing RGD or a non-peptidic RGD mimic.

26. (canceled)

27. The method of claim 1 further comprising administering to the subject a therapeutic agent.

28. The method of claim 1, wherein the disease is a neuromuscular disease, neurodegenerative disease, brain and nerve tumors, Neurogenetic Diseases, Cognitive disorders, Familial dystonia, Neuroinfectious disease, neuropsychiatric disorders.

29. (canceled)

30. A method for decreasing blood-Central Nervous System (blood-CNS) barrier permeability for treating a disease in a subject, the method comprising administering to the subject an agent that promotes the vitronectin-integrin signaling at the blood-CNS barrier.

31. The method of claim 30, wherein the administration inhibits transcytosis in endothelial cells in the central nervous system (blood-CNS) barrier.

32. (canceled)

33. The method of claim 30, wherein the agent that promotes vitronectin-integrin signaling is a nucleic acid encoding vitronectin.

34. (canceled)

35. The method of claim 30, wherein the integrin is integrin α5.

36. The method of claim 30, wherein the agent that promotes integrin signaling is a nucleic acid encoding integrin α5.

37. (canceled)

38. A method for decreasing Blood-Central Nervous System (Blood-CNS) Barrier permeability for treating a disease in a subject, the method comprising administering to the subject focal adhesion kinase (FAK) inhibitor.

39. The method of claim 38, wherein the FAK inhibitor is PF-562271.

40. The method of claim 30, wherein the disease is retinal disease, neurodegenerative disease, acute injury of the CNS, neuroinfectious disease, primary and metastatic cancers of the CNS, autoimmune disease of the CNS, neuroinflammatory conditions, or cognitive disorder.

41.-54. (canceled)
